(19) United States
(12) Patent Application Publication
(10) Pub. No.: US 2024/0293527 A1
(43) Pub. Date: Sep. 5, 2024

(54) DEOPTIMIZED YELLOW FEVER VIRUS AND METHODS AND USES THEREOF

(71) Applicant: Codagenix Inc., Farmingdale, NY (US)

(72) Inventors: John Robert Coleman, Blauvelt, NY (US); Steffen Mueller, Great Neck, NY (US); Ying Wang, South Setauket, NY (US); Chen Yang, Plainview, NY (US)

(73) Assignee: Codagenix Inc., Farmingdale, NY (US)

(21) Appl. No.: 18/572,482

(22) PCT Filed: Jul. 7, 2022
(86) PCT No.: PCT/US2022/073497

§ 371 Date: Dec. 20, 2023

Related U.S. Application Data
(60) Provisional application No. 63/219,256, filed on Jul. 7, 2021.
(60) Provisional application No. 63/339,114, filed on May 6, 2022.

(51) Int. Cl. A61K 39/12; A61K 39/00; A61P 37/04; C07K 14/005

(52) U.S. Cl. CPC A61K 39/12; A61P 37/04; C07K 14/005; A61K 2039/5254; A61K 2039/545; A61K 2039/585; C12N 2770/24122; C12N 2770/24134; C12N 2770/24162; C12N 2770/24171

(57) ABSTRACT

The present invention relates to deoptimized Yellow Fever viruses and their uses for the treatment of various forms of malignant tumors, and as vaccines against Yellow Fever. The method of the present invention is particularly useful for the treatment of malignant tumors in various organs, such as: breast, skin, colon, bronchial passage, epithelial lining of the gastrointestinal, upper respiratory and genito-urinary tracts, liver, prostate and the brain.

### CROSS-REFERENCE TO RELATED APPLICATIONS

This application includes a claim of priority under to U.S. provisional patent application Nos. 63/219,256, filed Jul. 7, 2021, and 63/339,114, filed May 6, 2022, the entirety of both are hereby incorporated by reference.

### REFERENCE TO SEQUENCE LISTING

This application contains a Sequence Listing submitted as an electronic xml file named “SequenceListing_064955_000050W000_ST26”, having a size in bytes of 70,255 bytes, and created on Jul. 5, 2022. The information contained in this electronic file is hereby incorporated by reference in its entirety.

### FIELD OF INVENTION

The present invention relates to deoptimized Yellow Fever virus vaccine strain and methods of using deoptimized Yellow Fever virus vaccine strain, as immune compositions and to induce oncolytic effects on malignant tumors and to treat malignant tumors.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Yellow Fever Virus

Tropical diseases of viral etiology such as Yellow Fever (YF) pose significant health risks to many people in the world, including service members as the US continues to deploy troops overseas. YF has a high mortality and lacks a specific therapy.

All current YF vaccines are based on the 17D live attenuated strain of YF virus. Although highly effective, the 17D vaccines are frequently reactogenic, with side effects including headache, myalgia, low grade fever and chills for 5-10 days following vaccination. In addition, very rare but serious vaccine-related adverse events and deaths have been documented. Since current 17D vaccines must be manufactured in chicken embryos and cannot be purified to modern standards, some of the observed reactogenicity may be a result of the high levels of egg and chicken embryo derived by-products in the vaccines. Furthermore, owing to their nature as biological isolates and their long passage history, 17D vaccine seeds are genetically poorly defined swarms of quasispecies mutants. Some more virulent mutants present in the vaccines at a low frequency may have an outsized effect on the reactogenicity of the vaccine.

The development of a new YF vaccine that is safer, less reactogenic, and manufacturable in modern cell culture systems is vital to provide protection to the global population.

### Synthetic Virology

Rapid improvements in DNA synthesis technology promise to revolutionize traditional methods employed in virology. One of the approaches traditionally used to eliminate the functions of different regions of the viral genome makes extensive but laborious use of site-directed mutagenesis to explore the impact of small sequence variations in the genomes of virus strains. However, viral genomes, especially of RNA viruses, are relatively short, often less than 10,000 bases long, making them amenable to whole genome synthesis using currently available technology. Recently developed microfluidic chip-based technologies can perform de novo synthesis of new genomes designed to specification for only a few hundred dollars each. This permits the generation of entirely novel coding sequences or the modulation of existing sequences to a degree practically impossible with traditional cloning methods. Such freedom of design provides tremendous power to perform large-scale redesign of DNA/RNA coding sequences to: (1) study the impact of changes in parameters such as codon bias, codon-pair bias, and RNA secondary structure on viral translation and replication efficiency; (2) perform efficient full genome scans for unknown regulatory elements and other signals necessary for successful viral reproduction; (3) develop new biotechnologies for genetic engineering of viral strains and design of anti-viral vaccines; (4) synthesize deoptimized viruses for use in oncolytic therapy.

### De Novo Synthesis of Viral Genomes

Computer-based algorithms are used to design and synthesize viral genomes de novo. These synthesized genomes, exemplified by the synthesis of Yellow Fever virus 17D, can be used to treat cancer.

It has been known that malignant tumors result from the uncontrolled growth of cells in an organ. The tumors grow to an extent where normal organ function may be critically impaired by tumor invasion, replacement of functioning tissue, competition for essential resources and, frequently, metastatic spread to secondary sites. Malignant cancer is the second leading cause of mortality in the United States.

Prior art methods for treating malignant tumors include surgical resection, radiation and/or chemotherapy. However, numerous malignancies respond poorly to all traditionally available treatment options and there are serious adverse side effects to the known and practiced methods. There has been much advancement to reduce the severity of the side effects while increasing the efficiency of commonly practiced treatment regimens. However, many problems remain, and there is a need to search for alternative modalities of treatment.

In recent years, there have been proposals to use viruses for the treatment of cancer: (1) as gene delivery vehicles; (2) as direct oncolytic agents by using viruses that have been genetically modified to lose their pathogenic features; or (3) as agents to selectively damage malignant cells using viruses which have been genetic engineered for this purpose.

Examples for the use of viruses against malignant gliomas include the following. Herpes Simplex Virus dlsptk (HSVdlsptk), is a thymidine kinase (TK)-negative mutant of HSV. This virus is attenuated for neurovirulence because of a 360-base-pair deletion in the TK gene, the product of which is necessary for normal viral replication. It has been found that HSVdlsptk retains propagation potential in rapidly dividing malignant cells, causing cell lysis and death. Unfortunately, all defective herpes viruses with attenuated neuropathogenicity have been linked with serious symptoms of encephalitis in experimental animals. For example, in mice infected intracerebrally with HSVdlsptk, the LD₅₀ Ic (intracranial administration) is 10⁶ pfu, a rather low dose. This limits the use of this mutant HSV. Other mutants of HSV have been proposed and tested. Nevertheless, death from viral encephalitis remains a problem.

Another proposal was to use retroviruses engineered to contain the HSV tk gene to express thymidine kinase which causes in vivo phosphorylation of nucleoside analogs, such as gancyclovir or acyclovir, blocking the replication of DNA and selectively killing the dividing cell. Izquierdo, M., et al., Gene Therapy, 2:66-69 (1995) reported the use of Moloney Murine Leukemia Virus (MoMLV) engineered with an insertion of the HSV tk gene with its own promoter. Follow-up of patients with glioblastomas that were treated with intraneoplastic inoculations of therapeutic retroviruses by MRI revealed shrinkage of tumors with no apparent short-term side effects. However, the experimental therapy had no effect on short-term or long-term survival of affected patients. Retroviral therapy is typically associated with the danger of serious long-term side effects (e.g., insertional mutagenesis).

Similar systems have been developed to target malignancies of the upper airways, tumors that originate within the tissue naturally susceptible to adenovirus infection and that are easily accessible. However, Glioblastoma multiforme, highly malignant tumors composed of widely heterogeneous cell types (hence the denomination multiforme) are characterized by exceedingly variable genotypes and are unlikely to respond to oncolytic virus systems directed against homogeneous tumors with uniform genetic abnormalities.

The effects of the virus modification described herein can be confirmed in ways that are known to one of ordinary skill in the art. Non-limiting examples induce plaque assays, growth measurements, reverse genetics of RNA viruses, and reduced lethality in test animals. The instant application demonstrates that the deoptimized viruses are capable of inducing protective immune responses in a host as well as inducing an anti-tumor response in the host.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a polynucleotide comprising a polynucleotide encoding one or more viral proteins or one or more fragments thereof of a parent Yellow Fever virus (YFV): wherein the polynucleotide is recoded compared to its parent YFV, wherein the amino acid sequence of the one or more viral proteins, or the one or more fragments thereof of the parent YFV encoded by the polynucleotide remains the same, or wherein the amino acid sequence of the one or more viral proteins or the one or more fragments thereof of the parent YFV encoded by the polynucleotide comprises one or more amino acid substitutions, additions, or deletions.

In various embodiments, the one or more viral proteins or one or more fragments thereof can be the E protein or a fragment thereof.

In various embodiments, the E protein or a fragment thereof can be encoded by a polynucleotide having SEQ ID NOs:7, 3, 9, 8, 4, 5 or 6, or a fragment thereof, or a variant of a polynucleotide having SEQ ID NOs:7, 3, 9, 8, 4, 5 or 6, or a fragment thereof. In various embodiments, the E protein or a fragment thereof can be encoded by a polynucleotide having SEQ ID NO:7. In various embodiments, the E protein or a fragment thereof can be encoded by a polynucleotide having SEQ ID NO:3. In various embodiments, the E protein or a fragment thereof can be encoded by a polynucleotide having SEQ ID NO:9. In various embodiments, the E protein or a fragment thereof can be encoded by a polynucleotide having SEQ ID NO:8. In various embodiments, the polynucleotide sequence can have SEQ ID NOs:7, 3, 9, 8, 4, 5 or 6.

In various embodiments, the parent YFV can be YFV strain 17D (YFV 17D), or has at least 95%, 96%, 97%, 98%, or 99% sequence identity to YFV 17D.

In various embodiments, the parent YFV can be YFV 17D-204, YFV 17DD, or YFV 17D-213, or has at least 95%, 96%, 97%, 98%, or 99% sequence identity to YFV 17D-204, YFV 17DD, or YFV 17D-213.

Various embodiments provide for a polypeptide encoded by any one of the polynucleotides of present invention as discussed herein.

Various embodiments provide for a deoptimized Yellow Fever virus (YFV) comprising any one of the polynucleotides of present invention as discussed herein.

Various embodiments provide for a deoptimized Yellow Fever virus (YFV) comprising a polypeptide encoded by any one of the polynucleotides of present invention as discussed herein.

Various embodiments provide for a deoptimized Yellow Fever virus (YFV) of the present invention, wherein expression of one or more of its viral proteins is reduced compared to its parent YFV.

Various embodiments provide for an immune composition or vaccine composition comprising a deoptimized YFV the present invention as discussed herein.

Various embodiments provide for a method of treating a malignant tumor or reducing tumor size, comprising: administering a deoptimized Yellow Fever virus (YFV) of the present invention as discussed herein or an immune composition of the present invention as discussed herein to a subject in need thereof.

Various embodiments provide for a method of treating a malignant tumor, comprising: administering a prime dose of deoptimized YFV of the present invention as discussed herein, or an immune composition of the present invention as discussed herein, to a subject in need thereof, and administering one or more boost dose of deoptimized YFV of the present invention as discussed herein, or an immune composition of the present invention as discussed herein, to the subject in need thereof.

Various embodiments provide for a method of reducing tumor size, comprising administering a prime dose of a deoptimized YFV of the present invention as discussed herein, or an immune composition of the present invention as discussed herein, to a subject in need thereof, and administering one or more boost dose of a deoptimized YFV of the present invention as discussed herein, or an immune composition of the present invention as discussed herein, to the subject in need thereof.

In various embodiments, the deoptimized YFV can be deoptimized YFV strain 17D (YFV 17D).

In various embodiments, the deoptimized YFV can be deoptimized YFV 17D-204, deoptimized YFV 17DD, or deoptimized YFV 17D-213.

In various embodiments, the prime dose can be administered subcutaneously, intramuscularly, intradermally, intranasally, or intravenously.

In various embodiments, the one or more boost dose can be administered intratumorally or intravenously.

In various embodiments, a first of the one or more boost dose can be administered about 2 weeks after one prime dose, or if more than one prime dose then about 2 weeks after the last prime dose.

In various embodiments, the subject can have cancer.

In various embodiments, the prime dose can be administered when the subject does not have cancer.

In various embodiments, the subject can be at a higher risk of developing cancer.

In various embodiments, the one or more boost dose can be administered about every 1, 2, 3, 4, 5, 6, 7, 8, 9 or 10 years after the prime dose when the subject does not have cancer.

In various embodiments, the subject can be subsequently diagnosed with cancer and the one or more boost dose can be administered after the subject is diagnosed with cancer.

In various embodiments, the method can further comprise administering a PD-1 inhibitor or a PD-L1 inhibitor. In various embodiments, the PD-1 inhibitor can be an anti-PD1 antibody. In various embodiments, the anti-PD1 antibody can be selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, and combinations thereof. In various embodiments, the PD-1 inhibitor can be selected from the group consisting of PF-06801591, anti-PD1 antibody expressing pluripotent killer T lymphocytes (PIK-PD-1), autologous anti-EGFRvIII 4SCAR-IgT cells, and combinations thereof. In various embodiments, the PD-L1 inhibitor can be an anti-PD-L1 antibody. In various embodiments, the anti-PD-L1 antibody can be selected from the group consisting of BGB-A333, CK-301, FAZ053, KN035, MDX-1105, MSB2311, SHR-1316, atezolizumab, avelumab, durvalumab, BMS-936559, CK-301, and combinations thereof. In various embodiments, the anti-PD-L1 inhibitor can be M7824.

In various embodiments, treating the malignant tumor can decrease the likelihood of recurrence of the malignant tumor. In various embodiments, treating the malignant tumor can decrease the likelihood of having a second cancer that is different from the malignant tumor.

In various embodiments, if the subject develops a second cancer that is different from the malignant tumor, the treatment of the malignant tumor can result in slowing the growth of the second cancer.

In various embodiments, after remission of the malignant tumor, if the subject develops a second cancer that is different from the malignant tumor, the treatment of the malignant tumor can result in in slowing the growth of the second cancer.

In various embodiments, treating the malignant tumor can stimulate an inflammatory immune response in the tumor. In various embodiments, treating the malignant tumor can recruit pro-inflammatory cells to the tumor. In various embodiments, treating the malignant tumor can stimulate an anti-tumor immune response.

In various embodiments, the malignant tumor can be a solid tumor. In various embodiments, the malignant tumor can be selected from a group consisting of glioma, neuroblastoma, glioblastoma multiforme, adenocarcinoma, medulloblastoma, mammary carcinoma, prostate carcinoma, colorectal carcinoma, hepatocellular carcinoma, bladder cancer, prostate cancer, lung carcinoma, bronchial carcinoma, epidermoid carcinoma, and melanoma.

In various embodiments, the deoptimized YFV can be administered intratumorally, intravenously, intracerebrally, intramuscularly, intraspinally or intrathecally.

In various embodiments, administering the deoptimized YFV can cause cell lysis in the tumor cells.

Various embodiments provide for a method of eliciting an immune response in a subject in need thereof, comprising administering a deoptimized Yellow Fever virus (YFV) of the present invention, or an immune or vaccine composition of the present invention, to a subject in need thereof. In various embodiments, the method elicits an immune response against YFV.

Various embodiments provide for a method of eliciting an immune response in a subject, comprising: administering a prime dose of deoptimized YFV of the present invention, or an immune or vaccine composition of the present invention, to a subject in need thereof, and administering one or more boost dose of deoptimized YFV of the present invention, or an immune or vaccine composition of the present invention, to the subject in need thereof. In various embodiments, the method elicits an immune response against YFV.

In various embodiments, the prime dose can be administered subcutaneously, intramuscularly, intradermally, intranasally, or intravenously. In various embodiments, the one or more boost dose can be administered subcutaneously, intramuscularly, intradermally, intranasally, or intravenously.

In various embodiments, a first of the one or more boost dose can be administered about 2 weeks after one prime dose, or if more than one prime dose then about 2 weeks after the last prime dose.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

Exemplary embodiments are illustrated in referenced figures. It is intended that the embodiments and figures disclosed herein are to be considered illustrative rather than restrictive.

FIG. 1 depict various representative versions of the codon-pair deoptimized (CPD) Yellow Fever 17D Viral Genome design.

FIGS. 2A-2C depict a schematic representation of the eight 17D-WWDW genome DNA fragments used in overlapping PCR. 2A. Schematic showing the boundaries of the eight genome fragments. F2 contains the CPD sequences. The green box at the 5′-end of the full-length genome DNA represents the phi-2.5 T7 promoter. 2B. Sizes of eight fragments generated by PCR. 2C. Gel image of the 8 genome fragments amplified from 8 different plasmids.

FIG. 3A depicts a PCR gel check for F1-F8 for construction of the deoptimized YFV. F2 can be either of the wild-type (Wt) or any one of CPD-fragments (DW, WD, DD, or DDDW).

FIG. 3B depicts 17D-WWDW vaccine candidate RT-PCR fragments from Passages 5, 12, and 15 (top, middle, bottom).

FIG. 3C depicts all 8 PCR fragments generated from 17D-WWDW vaccine candidate from viremia sample #18164.

FIG. 4 depicts gel check for four full length CPD YF genome PCR (˜11 kb).

FIG. 5 depicts RNA gel check for four full length YF-CPD genome RNAs.

FIG. 6 plaque assay for the vaccine strain YF-17D (left column) and the recovered YF-DW viral variant (right column) at 33° C. (top row) and 37° C. (bottom row).

FIG. 7 depicts plaque assay for the vaccine strain YF-(left column) and the recovered YF-DDDW viral variant (right column) at 33° C. (top row) and 37° C. (bottom row).

FIGS. 8A-8D depict detection of Infected Vero Cells by Immunohistochemical Staining. Cells transfected with (8A) YF-DD RNA or (8B) no RNA were fixed with Methanol/Acetone 8 days after RNA transfection. Cells infected with (8C) day 4 YF-DD transfection supernatants or (8D) mock supernatant were fixed with Methanol/Acetone 8 days after infection. YF-infected cells were visualized by IHC staining with mouse mAb anti-Flavivirus Group Antigen, clone D1-4G2-4-15 (ATCC® HB-112), in conjunction with HRP-labeled goat anti-mouse secondary antibody and VECTOR VIP chromogenic substrate.

FIG. 9 depicts the gel image of the full-length 17D-WWDW genome DNA constructed by overlapping PCR of 8 genome fragments shown in FIG. 2.

FIG. 10 depicts in vitro transcript of the full-length 17D-WWDW genome RNA generated from overlapping PCR was checked on agarose gel. Ctrl RNA represents RNA used to control for presence of RNase in the gel.

FIG. 11 depicts plaque morphology of the parental 17D and deoptimized 17D-WWDW virus on Vero cells for 5 days at 37° C. Viral titers are 8×10⁵ PFU/ml for parental 17D (9th freeze-thaw cycle) and 6.25×10⁷ PFU/ml for 17D-WWDW.

FIG. 12 depicts plaque morphology of the parental 17D and deoptimized 17D-WWDW virus on Vero cells for 5 days at 33° C. Viral titers were 8×10⁵ PFU/ml for parental 17D (9th freeze-thaw cycle) and 7.75×10⁷ PFU/ml for 17D-WWDW.

FIG. 13 depicts neutralizing antibody titers against 17D in monkey sera from Southern Research Study 16128.02. Results are shown as top: mean±SEM, bottom: geomean±geoSD. *p=0.0001, ns; no statistically significant difference, p>0.05. Values below the lowest dilution (<10) were entered as 9 on the graph.

FIG. 14 depicts post-vaccination viremia in NHP serum, as assessed by qRT-PCR.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, outbred or inbred strains of laboratory mice, and athymic nude mice. In a preferred embodiment, the subject is a human. Preferred embodiments of birds are domesticated poultry species, including, but not limited to, chickens, turkeys, ducks, and geese.

A “viral host” means any animal or artificially modified animal, or insect that a virus can infect. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In a specific embodiment, the viral host is a human. Embodiments of birds are domesticated poultry species, including, but not limited to, chickens, turkeys, ducks, and geese. Insects include, but are not limited to mosquitos.

“Parent virus” as used herein refer to a reference virus to which a recoded nucleotide sequence is compared for encoding the same or similar amino acid sequence.

“Frequently used codons” or “codon usage bias” as used herein refer to differences in the frequency of occurrence of synonymous codons in coding DNA for a particular species, for example, human, or Yellow Fever Virus.

“Codon pair bias” as used herein refers to synonymous codon pairs that are used more or less frequently than statistically predicted in a particular species, for example, human, or Yellow Fever Virus.

“Corresponding sequence” as used herein refers to a comparison sequence by which the deoptimized sequence is encoding the same or similar amino acid sequence of the comparison sequence. In various embodiments, the corresponding sequence is a sequence that encodes a viral protein. In various embodiments, the corresponding sequence is at least 50 codons in length. In various embodiments, the corresponding sequence is at least 100 codons in length. In various embodiments, the corresponding sequence is at least 150 codons in length. In various embodiments, the corresponding sequence is at least 200 codons in length. In various embodiments, the corresponding sequence is at least 250 codons in length. In various embodiments, the corresponding sequence is at least 300 codons in length. In various embodiments, the corresponding sequence is at least 350 codons in length. In various embodiments, the corresponding sequence is at least 400 codons in length. In various embodiments, the corresponding sequence is at least 450 codons in length. In various embodiments, the corresponding sequence is at least 500 codons in length. In various embodiments, the corresponding sequence is the viral protein sequence. In various embodiments, the corresponding sequence is the sequence of the entire virus.

In various embodiments, “similar amino acid sequence” as used herein refers to an amino acid sequence having less than 2% amino acid substitutions, deletions or additions compared to the comparison sequence. In various embodiments, if specifically provided for in the claims, “similar amino acid sequence” refers to an amino acid sequence having less than 1.75% amino acid substitutions, deletions or additions compared to the comparison sequence. In various embodiments, if specifically provided for in the claims, “similar amino acid sequence” refers to an amino acid sequence having less than 1.5% amino acid substitutions, deletions or additions compared to the comparison sequence. In various embodiments, if specifically provided for in the claims, “similar amino acid sequence” refers to an amino acid sequence having less than 1.25% amino acid substitutions, deletions or additions compared to the comparison sequence. In various embodiments, if specifically provided for in the claims, “similar amino acid sequence” refers to an amino acid sequence having less than 1% amino acid substitutions, deletions or additions compared to the comparison sequence. In various embodiments, if specifically provided for in the claims, “similar amino acid sequence” refers to an amino acid sequence having less than 0.75% amino acid substitutions, deletions or additions compared to the comparison sequence. In various embodiments, if specifically provided for in the claims, “similar amino acid sequence” refers to an amino acid sequence having less than 0.5% amino acid substitutions, deletions or additions compared to the comparison sequence. In various embodiments, if specifically provided for in the claims, “similar amino acid sequence” refers to an amino acid sequence having less than 0.25% amino acid substitutions, deletions or additions compared to the comparison sequence.

Described herein, the inventors used their Synthetic Attenuated Virus Engineering (SAVE) platform to design, generate and test live attenuated vaccine candidates against YF. SAVE is a computational, synthetic biology tool for rapid generation of live-attenuated vaccines. The SAVE computer algorithms provide for the design of virus attenuation by recoding viral genomes rich in underrepresented human codons and codon-pairs without changing the amino acid sequence—a process termed virus deoptimization. The redesigned genome is synthesized de novo and transfected into cells to recover the attenuated, genetically defined, human-cell-deoptimized vaccine strain. The resulting viruses are fully immunogenic, preserving all or essential the epitopes of target virus, but are orders of magnitude attenuated, due to a slowing of viral gene expression in the infected host cell. In various embodiments, resulting viruses are designed or adapted to have particular mutations.

Starting with the published consensus sequence of the current 17D vaccine YF-VAX, we designed and synthesized novel deoptimized vaccine strains, for example, 17D-WWDW and 17D-WD, and each with increasing levels of attenuation over the 17D parental phenotype. One of our candidates, 17D-WWDW, grows to high titers in mammalian cell culture (Vero), is genetically stable for at least 15 passages, and is as immunogenic in Rhesus macaques as the highly effective parental 17D vaccine.

Live attenuated, codon pair deoptimized vaccine candidates against yellow fever, 17D-WWDW and 17D-WD, were successfully recovered following genome reconstruction via overlapping PCR and transfection. 17D-WWDW was genetically stable over 15 passages in Vero cells.

Both deoptimized vaccines were safe and immunogenic in NHPs. Two SC injections of each vaccine tested in the study were well tolerated in the monkeys, and there was no apparent difference in the in-life parameters between the two deoptimized vaccines and the 17D reference vaccine. 17D-WWDW elicited similar levels of neutralizing antibodies to 17D reference vaccine after one dose. Absence of viremia following boost suggests that vaccinated animals were protected from the surrogate challenge posed by the vaccine booster dose. Taken together, these findings suggest that 17D-WWDW is a candidate for an improved, scalable, genetically stable, live attenuated vaccine against YF.

Additionally, we have shown, for example, in International Patent Application No. PCT/US2020/032901, herein incorporated by reference as though fully set forth, that Yellow Fever Virus, particularly, synthetic Yellow Fever Virus Strain 17D can be used for the treatment of cancer. Described herein we develop live attenuated Yellow Fever vaccine candidates that allow manufacture in modern cell culture systems (rather than chicken embryos), while preserving or increasing and stabilizing the attenuation phenotype relative to the current 17D vaccine. To this end codon pair deoptimized cassettes are introduced into the 17D viral genome by reverse genetics methods to “over-attenuate” the resulting vaccine candidate. The over-attenuation provides a safety “buffer” that will allow to absorb potential de-attenuating effects of mutations that may occur upon virus adaptation when switching the manufacturing substrate of the vaccine from chick embryos to cell culture.

### Virus Composition, Oncolytic Virus Composition and Pharmaceutical Compositions

Embodiments of the present invention provide for a deoptimized Yellow Fever virus, wherein the E protein coding sequence is deoptimized. Various embodiments of the present invention provide for a pharmaceutical composition comprising a deoptimized Yellow Fever virus wherein the E protein coding sequence is deoptimized and a pharmaceutically acceptable carrier or excipient. In various embodiments, the pharmaceutically acceptable carrier or excipient is sorbitol or gelatin, which can be used as stabilizers. In various embodiments, the composition comprising the deoptimized Yellow Fever virus wherein the E protein coding sequence is deoptimized (e.g., vaccine preparation) can be lyophilized and kept under cold-chain conditions.

In various embodiments, the pharmaceutically acceptable carrier or excipient is particularly adapted for delivery of the deoptimized Yellow Fever virus wherein the E protein coding sequence is deoptimized for cancer treatment; for example, to enhance delivery to the tumor site. Examples of these carriers include but are not limited to carbon nanotube, layered double hydroxide (LDH), iron oxide nanoparticles, mesoporous silica nanoparticles (MSN), polymeric nanoparticles, liposomes, micelle, protein nanoparticles, and dendrimer.

The deoptimized Yellow Fever virus is one which does not cause, or has less than a 0.01% chance of causing Yellow Fever in a mammalian subject and in particular in a human subject.

In various embodiments, the deoptimized Yellow Fever virus wherein the E protein coding sequence is deoptimized is the deoptimized form of Yellow Fever virus (YFV) 17D vaccine (e.g., UniProtKB—P03314 (POLG_YEFV1), as of Jun. 28, 2021, herein incorporated by reference as though fully set forth). In various embodiments, the deoptimized Yellow Fever virus wherein the E protein coding sequence is deoptimized is the deoptimized form of Yellow Fever virus (YFV) 17D (Genbank Accession #JN628279, as of Jun. 28, 2021, Stock et al., 2012; herein incorporated by reference as though fully set forth).

The attenuated live YFV 17D vaccine strain is derived from a wild-type YF virus (the Asibi strain) isolated in Ghana in 1927 and attenuated by serial passages in chicken embryo tissue culture. Two substrains of the 17D vaccine virus are currently used for vaccine production in embryonated chicken eggs, namely 17D-204 and 17DD. Some vaccines are also prepared from a distinct substrain of 17D-204 (17D-213). Thus, in various embodiments, the deoptimized YFV 17D wherein the E protein coding sequence is deoptimized, is deoptimized YFV 17D-204 wherein the E protein coding sequence is deoptimized, deoptimized YFV 17DD wherein the E protein coding sequence is deoptimized, or deoptimized YFV 17D-213 wherein the E protein coding sequence is deoptimized.

Various embodiments of the invention provide a deoptimized YFV virus, which comprises a deoptimized viral genome containing nucleotide substitutions engineered in one or multiple locations in the genome, wherein the substitutions introduce a plurality of synonymous codons into the genome (e.g., codon deoptimization) and/or a change of the order of existing codons for the same amino acid (change of codon pair utilization (e.g., codon-pair deoptimization)). In both cases, the original vaccine strain amino acid sequences are retained. In alternative embodiments, the original vaccine strain amino acid sequences are substantially retained; that is, there is one or more amino acid addition, deletion or substitution in comparison to the original vaccine strain's amino acid sequence. In various embodiments, one or more amino acid addition, deletion or substitution is up to 50 amino acid additions, deletions or substitutions. In various embodiments, one or more amino acid addition, deletion or substitution is up to 40 amino acid additions, deletions or substitutions. In various embodiments, one or more amino acid addition, deletion or substitution is up to 30 amino acid additions, deletions or substitutions. In various embodiments, one or more amino acid addition, deletion or substitution is up to 25 amino acid additions, deletions or substitutions. In various embodiments, one or more amino acid addition, deletion or substitution is up to 20 amino acid additions, deletions or substitutions. In various embodiments, one or more amino acid addition, deletion or substitution is up to 15 amino acid additions, deletions or substitutions. In various embodiments, one or more amino acid addition, deletion or substitution is up to 10 amino acid additions, deletions or substitutions. In various embodiments, one or more amino acid addition, deletion or substitution is up to 5 amino acid additions, deletions or substitutions.

In various embodiments, a continuous segment of an E protein is recoded, wherein the continuous segment is about ¾ the length of the E protein. In various embodiments, a continuous segment of E protein is recoded, wherein the continuous segment is about ½ the length of the E protein. In various embodiments, a continuous segment of an E protein is recoded, wherein the continuous segment is about ⅓ the length of the E protein. In various embodiments, a continuous segment of an E protein is recoded, wherein the continuous segment is about ¼ the length of the E protein. In various embodiments, a continuous segment of an E protein is recoded, wherein the continuous segment is about ⅕ the length of the viral protein.

In various embodiments, a continuous segment of an E protein is recoded, wherein the continuous segment is about 10-20% of the length of the E protein. In various embodiments, a continuous segment of an E protein is recoded, wherein the continuous segment is about 20-30% of the length of the E protein. In various embodiments, a continuous segment of an E protein is recoded, wherein the continuous segment is about 25-35% of the length of the E protein. In various embodiments, a continuous segment of an E protein is recoded, wherein the continuous segment is about 30-40% of the length of the E protein. In various embodiments, a continuous segment of an E protein is recoded, wherein the continuous segment is about 35-45% of the length of the E protein. In various embodiments, a continuous segment of an E protein is recoded, wherein the continuous segment is about 40-50% of the length of the E protein. In various embodiments, a continuous segment of an E protein is recoded, wherein the continuous segment is about 45-55% of the length of the E protein. In various embodiments, a continuous segment of an E protein is recoded, wherein the continuous segment is about 50-60% of the length of the E protein. In various embodiments, a continuous segment of an E protein is recoded, wherein the continuous segment is about 55-65% of the length of the E protein. In various embodiments, a continuous segment of an E protein is recoded, wherein the continuous segment is about 60-70% of the length of the E protein. In various embodiments, a continuous segment of an E protein is recoded, wherein the continuous segment is about 70-80% of the length of the E protein.

Various embodiments of the invention provide for a codon deoptimized yellow fever virus wherein the E protein coding sequence is deoptimized.

In various embodiments, the codon deoptimized yellow fever virus comprises at least 10 deoptimized codons in an E protein coding sequence, wherein the at least 10 deoptimized codons are each a synonymous codon less frequently used in the yellow fever virus. In various embodiments, the codon deoptimized yellow fever virus comprises at least 20, 30, 40, 50, 60, 70, 80, 90, 100, 125, 150, 200, 250, 300, or 400 deoptimized codons in the E protein coding sequence, wherein the at least 20, 30, 40, 50, 60, 70, 80, 90, 100, 125, 150, 200, 250, 300, or 400 deoptimized codons are each a synonymous codon less frequently used in the yellow fever virus. The synonymous codon less frequently used in the yellow fever virus is a codon that encodes the same amino acid, but the codon is an unpreferred or less preferred codon by the yellow fever virus for the amino acid.

TABLE 1
Yellow Fever Virus (17D Strain) Codon Usage

| Amino | | # of | Amino | | # of | Amino | | # of | Amino | | # of |
|---|---|---|---|---|---|---|---|---|---|---|---|
| acid | Codon | Use | acid | Codon | Use | acid | Codon | Use | acid | Codon | Use |
| Phe | UUU | 64 | Ser | UCU | 44 | Tyr | UAU | 35 | Cys | UGU | 32 |
| | UAC | 52 | | UCC | 42 | | UAC | 44 | | UGC | 32 |
| Leu | UUA | 8 | | UCA | 56 | Ochre | UAA | 1 | Opal | UGA | 0 |
| | UUG | 72 | | UCG | 8 | Amber | UAG | 0 | Trp | UGG | 85 |
| Leu | CUU | 46 | Pro | CCU | 40 | His | CAU | 50 | Arg | CGU | 12 |
| | CUC | 49 | | CCC | 28 | | CAC | 32 | | CGC | 25 |
| | CUA | 37 | | CCA | 56 | Gln | CAA | 42 | | CGA | 12 |
| | CUG | 100 | | CCG | 12 | | CAG | 51 | | CGG | 11 |
| Ile | AUU | 63 | Thr | ACU | 53 | Asn | AAU | 56 | Ser | AGU | 34 |
| | AUC | 69 | | ACC | 51 | | AAC | 68 | | AGC | 31 |
| | AUA | 44 | | ACA | 73 | Lys | AAA | 92 | Arg | AGA | 67 |
| Met | AUG | 129 | | ACG | 21 | | AAG | 10 | | AGG | 83 |
| Val | GUU | 69 | Ala | GCU | 83 | Asp | GAU | 70 | Gly | GGU | 36 |
| | GUC | 69 | | GCC | 80 | | GAC | 88 | | GGC | 68 |
| | GUA | 16 | | GCA | 58 | Glu | GAA | 108 | | GGA | 124 |
| | GUG | 132 | | GCG | 23 | | GAG | 102 | | GGG | 73 |

Codon usage for the yellow fever virus, 17D strain, long open reading frame of 10,233 nucleotides (3411 codons excluding the termination codon). Data from Rice et al. (1985)

In various embodiments, the codon deoptimized yellow fever virus comprises at least 10 deoptimized codons in an E protein coding sequence, wherein the at least 10 deoptimized codons are each a synonymous codon less frequently used in the viral host, such as in humans. In various embodiments, the codon deoptimized yellow fever virus comprises at least 20, 30, 40, 50, 60, 70, 80, 90, 100, 125, 150, 200, 250, 300, or 400 deoptimized codons in the E protein coding sequence, wherein the at least 20, 30, 40, 50, 60, 70, 80, 90, 100, 125, 150, 200, 250, 300 or 400 deoptimized codons are each a synonymous codon less frequently used in the viral host, such as humans. The synonymous codon less frequently used in in the viral host is a codon that encodes the same amino acid, but the codon is an unpreferred codon or less preferred by that viral host for the amino acid. The synonymous codon less frequently used in humans is a codon that encodes the same amino acid, but the codon is an unpreferred codon or less preferred by humans for the amino acid.

In various embodiments, the codon deoptimized yellow fever virus has its E protein coding codons deoptimized and has the same amino acid sequence as YFV 17D, YFV 17D-204, YFV 17DD, or YFV 17D-213. In various embodiments, the codon deoptimized yellow fever virus has its E protein coding codons deoptimized and has up to 1, 2, 3, 4 or 5 amino acid changes as compared to the amino acid sequence as YFV 17D, YFV 17D-204, YFV 17DD, or YFV 17D-213. An amino acid change can be a different amino acid, a deletion of an amino acid, or an addition of an amino acid.

In various embodiments, a continuous segment of an E protein is recoded, wherein the continuous segment is about ¾ the length of the E protein. In various embodiments, a continuous segment of E protein is recoded, wherein the continuous segment is about ½ the length of the E protein. In various embodiments, a continuous segment of an E protein is recoded, wherein the continuous segment is about ⅓ the length of the E protein. In various embodiments, a continuous segment of an E protein is recoded, wherein the continuous segment is about ¼ the length of the E protein. In various embodiments, a continuous segment of an E protein is recoded, wherein the continuous segment is about ⅕ the length of the viral protein.

In various embodiments, a continuous segment of an E protein is recoded, wherein the continuous segment is about 10-20% of the length of the E protein. In various embodiments, a continuous segment of an E protein is recoded, wherein the continuous segment is about 20-30% of the length of the E protein. In various embodiments, a continuous segment of an E protein is recoded, wherein the continuous segment is about 25-35% of the length of the E protein. In various embodiments, a continuous segment of an E protein is recoded, wherein the continuous segment is about 30-40% of the length of the E protein. In various embodiments, a continuous segment of an E protein is recoded, wherein the continuous segment is about 35-45% of the length of the E protein. In various embodiments, a continuous segment of an E protein is recoded, wherein the continuous segment is about 40-50% of the length of the E protein. In various embodiments, a continuous segment of an E protein is recoded, wherein the continuous segment is about 45-55% of the length of the E protein. In various embodiments, a continuous segment of an E protein is recoded, wherein the continuous segment is about 50-60% of the length of the E protein. In various embodiments, a continuous segment of an E protein is recoded, wherein the continuous segment is about 55-65% of the length of the E protein. In various embodiments, a continuous segment of an E protein is recoded, wherein the continuous segment is about 60-70% of the length of the E protein. In various embodiments, a continuous segment of an E protein is recoded, wherein the continuous segment is about 70-80% of the length of the E protein.

Methods of codon deoptimization are described in International Application No. PCT/US2005/036241, the contents of which are herein incorporated by reference.

Various embodiments of the invention provide for a codon-pair deoptimized (CPD) yellow fever virus wherein the E protein coding sequence is codon-pair deoptimized.

In various embodiments, the codon-pair deoptimized yellow fever virus wherein the E protein coding sequence is codon-pair deoptimized comprises a reduction in codon-pair bias (CPB) as compared to the yellow fever virus before codon-pair deoptimization of the yellow fever virus. Thus, the codon-pair deoptimized yellow fever virus comprises rearranging existing codons in the E protein coding sequence. Rearranging existing codons in the E protein coding sequence comprises substituting a codon pair with a codon pair that has a lower codon-pair score.

As such, it comprises recoded E protein coding sequences wherein each sequence has existing synonymous codons from its parent E protein-coding sequence in a rearranged order and has a CPB less than the CPB of the parent E protein-coding sequence from which it is derived.

In some embodiments, a subset of codon pairs is substituted by rearranging a subset of synonymous codons. In other embodiments, codon pairs are substituted by maximizing the number of rearranged synonymous codons. It is noted that while rearrangement of codons leads to codon-pair bias that is reduced (made more negative) for the virus coding sequence overall, and the rearrangement results in a decreased codon pair scores (CPS) at many locations, there may accompanying CPS increases at other locations, but on average, the codon pair scores, and thus the CPB of the deoptimized sequence, is reduced.

In various embodiments, the CPB is reduced by at least 0.01, at least 0.02, at least 0.03, at least 0.04, at least 0.05, at least 0.10, at least 0.15, at least 0.20, at least 0.25, at least 0.30, at least 0.35, at least 0.40, at least 0.45 or at least 0.50 compared to the corresponding sequence. In certain embodiments, it is in comparison corresponding sequence from which the calculation is to be made; for example, the corresponding sequence of a wild type virus; or in another example, the corresponding sequence of 17D YFV.

In certain embodiments, the codon pair bias of the recoded sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence. In certain embodiments, it is in comparison corresponding sequence from which the calculation is to be made; for example, the corresponding sequence of a wild type virus; or in another example, the corresponding sequence of 17D YFV.

In embodiments wherein the deoptimized sequence comprises a recoded sequence having reduced codon pair bias compared to a corresponding sequence, the recoded sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In various embodiments, the codon pair bias is based on codon pair usage in yellow fever virus. In various embodiments, the codon pair bias is based on codon pair usage in humans.

In various embodiments, the codon-pair deoptimized yellow fever virus wherein the E protein coding sequence is codon-pair deoptimized has the same amino acid sequence as YFV 17D, YFV 17D-204, YFV 17DD, or YFV 17D-213. In various embodiments, the codon-pair deoptimized yellow fever wherein the E protein coding sequence is codon-pair deoptimized virus has up to 1, 2, 3, 4, or 5 amino acid changes as compared to the amino acid sequence as YFV 17D, YFV 17D-204, YFV 17DD, or YFV 17D-213. An amino acid change can be a different amino acid, a deletion of an amino acid, or an addition of an amino acid.

In various embodiments, a continuous segment of an E protein is recoded, wherein the continuous segment is about ¾ the length of the E protein. In various embodiments, a continuous segment of E protein is recoded, wherein the continuous segment is about ½ the length of the E protein. In various embodiments, a continuous segment of an E protein is recoded, wherein the continuous segment is about ⅓ the length of the E protein. In various embodiments, a continuous segment of an E protein is recoded, wherein the continuous segment is about ¼ the length of the E protein. In various embodiments, a continuous segment of an E protein is recoded, wherein the continuous segment is about ⅕ the length of the viral protein.

In various embodiments, a continuous segment of an E protein is recoded, wherein the continuous segment is about 10-20% of the length of the E protein. In various embodiments, a continuous segment of an E protein is recoded, wherein the continuous segment is about 20-30% of the length of the E protein. In various embodiments, a continuous segment of an E protein is recoded, wherein the continuous segment is about 25-35% of the length of the E protein. In various embodiments, a continuous segment of an E protein is recoded, wherein the continuous segment is about 30-40% of the length of the E protein. In various embodiments, a continuous segment of an E protein is recoded, wherein the continuous segment is about 35-45% of the length of the E protein. In various embodiments, a continuous segment of an E protein is recoded, wherein the continuous segment is about 40-50% of the length of the E protein. In various embodiments, a continuous segment of an E protein is recoded, wherein the continuous segment is about 45-55% of the length of the E protein. In various embodiments, a continuous segment of an E protein is recoded, wherein the continuous segment is about 50-60% of the length of the E protein. In various embodiments, a continuous segment of an E protein is recoded, wherein the continuous segment is about 55-65% of the length of the E protein. In various embodiments, a continuous segment of an E protein is recoded, wherein the continuous segment is about 60-70% of the length of the E protein. In various embodiments, a continuous segment of an E protein is recoded, wherein the continuous segment is about 70-80% of the length of the E protein.

Method of codon-pair deoptimization are described in International Patent Application No. PCT/US2008/058952, the contents of which are herein incorporated by reference.

Various embodiments of the invention provide for a deoptimized yellow fever virus, wherein the frequency of the CG and/or TA (or UA) dinucleotide content in the E protein coding sequence is altered. In various embodiments, the CpG dinucleotide content in the E protein coding sequence in the deoptimized YFV is increased. In various embodiments, the UpA dinucleotide content in the E protein coding sequence in the deoptimized YFV is increased.

For example, the increase is of about 15-55 CpG or UpA di-nucleotides compared the corresponding sequence. In various embodiments, increase is of about 15, 20, 25, 30, 35, 40, 45, or 55 CpG or UpA di-nucleotides compared the corresponding sequence. In some embodiments, the increased number of CpG or UpA di-nucleotides compared to a corresponding sequence is about 10-75, 15-25, 25-50, or 50-75 CpG or UpA di-nucleotides compared the corresponding sequence.

In various embodiments, the deoptimized yellow fever virus wherein the frequency of the CG and/or TA (or UA) dinucleotide content in the E protein coding sequence is altered has the same amino acid sequence as YFV 17D, YFV 17D-204, YFV 17DD, or YFV 17D-213. In various embodiments, the deoptimized yellow fever virus wherein the frequency of the CG and/or TA (or UA) dinucleotide content in the E protein coding sequence is altered has up to 1, 2, 3, 4, or 5 amino acid changes as compared to the amino acid sequence as YFV 17D, YFV 17D-204, YFV 17DD, or YFV 17D-213. An amino acid change can be a different amino acid, a deletion of an amino acid, or an addition of an amino acid.

In various embodiments, a continuous segment of an E protein is recoded, wherein the continuous segment is about ¾ the length of the E protein. In various embodiments, a continuous segment of E protein is recoded, wherein the continuous segment is about ½ the length of the E protein. In various embodiments, a continuous segment of an E protein is recoded, wherein the continuous segment is about ⅓ the length of the E protein. In various embodiments, a continuous segment of an E protein is recoded, wherein the continuous segment is about ¼ the length of the E protein. In various embodiments, a continuous segment of an E protein is recoded, wherein the continuous segment is about ⅕ the length of the viral protein.

In various embodiments, a continuous segment of an E protein is recoded, wherein the continuous segment is about 10-20% of the length of the E protein. In various embodiments, a continuous segment of an E protein is recoded, wherein the continuous segment is about 20-30% of the length of the E protein. In various embodiments, a continuous segment of an E protein is recoded, wherein the continuous segment is about 25-35% of the length of the E protein. In various embodiments, a continuous segment of an E protein is recoded, wherein the continuous segment is about 30-40% of the length of the E protein. In various embodiments, a continuous segment of an E protein is recoded, wherein the continuous segment is about 35-45% of the length of the E protein. In various embodiments, a continuous segment of an E protein is recoded, wherein the continuous segment is about 40-50% of the length of the E protein. In various embodiments, a continuous segment of an E protein is recoded, wherein the continuous segment is about 45-55% of the length of the E protein. In various embodiments, a continuous segment of an E protein is recoded, wherein the continuous segment is about 50-60% of the length of the E protein. In various embodiments, a continuous segment of an E protein is recoded, wherein the continuous segment is about 55-65% of the length of the E protein. In various embodiments, a continuous segment of an E protein is recoded, wherein the continuous segment is about 60-70% of the length of the E protein. In various embodiments, a continuous segment of an E protein is recoded, wherein the continuous segment is about 70-80% of the length of the E protein.

Methods of altering CG and/or TA (or UA) dinucleotide content are described in International Patent Application No. PCT/US2008/058952, the contents of which are herein incorporated by reference.

In various embodiments, the E protein of the deoptimized YFV is encoded by a polynucleotide having SEQ ID NO:3.

In various embodiments, the E protein of the deoptimized YFV is encoded by a variant of a polynucleotide having SEQ ID NO:3, wherein the variant is not the YFV 17D sequence or wild type sequence.

In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:3. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of a polynucleotide having SEQ ID NO:3.

In various embodiments, the deoptimized YFV is encoded by YFV 17D, wherein the YFV 17D E protein coding sequence is replaced with the E protein coding sequence variant of a polynucleotide having SEQ ID NO:3, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:3, wherein the variant is not the YFV 17D sequence. In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the E protein of the deoptimized YFV is encoded by a polynucleotide having SEQ ID NO:4.

In various embodiments, the E protein of the deoptimized YFV is encoded by a variant of a polynucleotide having SEQ ID NO:4, wherein the variant is not the YFV 17D sequence or wild type sequence.

In particular embodiments, variant of a polynucleotide having SEQ ID NO:4 has at least 95%, 96%, 97%, 98% or 99% sequence identity to SEQ ID NO:4 In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:4. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:4.

In various embodiments, the deoptimized YFV is encoded by YFV 17D, wherein the YFV 17D E protein coding sequence is replaced with the E protein coding sequence variant of a polynucleotide having SEQ ID NO:4, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:4, wherein the variant is not the YFV 17D sequence.

In particular embodiments, variant of a polynucleotide having SEQ ID NO:4 has at least 95%, 96%, 97%, 98% or 99% sequence identity to SEQ ID NO:4. In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:5.

In various embodiments, the E protein of the deoptimized YFV is encoded by a variant of a polynucleotide having SEQ ID NO:5, wherein the variant is not the YFV 17D sequence or wild type sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:5. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:5.

In various embodiments, the deoptimized YFV is encoded by YFV 17D, wherein the YFV 17D E protein coding sequence is replaced with the E protein coding sequence variant of a polynucleotide having SEQ ID NO:5, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:5, wherein the variant is not the YFV 17D sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:6.

In various embodiments, the E protein of the deoptimized YFV is encoded by a variant of a polynucleotide having SEQ ID NO:6, wherein the variant is not the YFV 17D sequence or wild type sequence.

In particular embodiments, variant of a polynucleotide having SEQ ID NO:6 has at least 95%, 96%, 97%, 98% or 99% sequence identity to SEQ ID NO:6 In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:6. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:6.

In various embodiments, the deoptimized YFV is encoded by YFV 17D, wherein the YFV 17D E protein coding sequence is replaced with the E protein coding sequence variant of a polynucleotide having SEQ ID NO:6, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:6, wherein the variant is not the YFV 17D sequence.

In particular embodiments, variant of a polynucleotide having SEQ ID NO:6 has at least 95%, 96%, 97%, 98% or 99% sequence identity to SEQ ID NO:6. In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:7 (YF-WWDW).

In various embodiments, the E protein of the deoptimized YFV is encoded by a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 95%, 96%, 97%, 98% or 99% sequence identity to SEQ ID NO: 7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:7 (YF-WWDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence SEQ ID NO:7 (YF-WWDW).

In various embodiments, the deoptimized YFV is encoded by YFV 17D, wherein the YFV 17D E protein coding sequence is replaced with an E protein coding sequence variant of a polynucleotide having SEQ ID NO:7 (YF-WWDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with an E protein coding sequence a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 950%, 96%, 97%, 98% or 99% sequence identity to SEQ ID NO: 7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:8.

In various embodiments, the E protein of the deoptimized YFV is encoded by a variant of a polynucleotide having SEQ ID NO:8, wherein the variant is not the YFV 17D sequence or wild type sequence.

In particular embodiments, variant of a polynucleotide having SEQ ID NO:8 has at least 95%, 96%, 97%, 98% or 99% sequence identity to SEQ ID NO:8 In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:8. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:8.

In various embodiments, the deoptimized YFV is encoded by YFV 17D, wherein the YFV 17D E protein coding sequence is replaced with the E protein coding sequence variant of a polynucleotide having SEQ ID NO:8, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:8, wherein the variant is not the YFV 17D sequence.

In particular embodiments, variant of a polynucleotide having SEQ ID NO:8 has at least 95%, 96%, 97%, 98% or 99% sequence identity to SEQ ID NO:8. In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:9.

In various embodiments, the E protein of the deoptimized YFV is encoded by a variant of a polynucleotide having SEQ ID NO:9, wherein the variant is not the YFV 17D sequence or wild type sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9.

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:9. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:9.

In various embodiments, the deoptimized YFV is encoded by YFV 17D, wherein the YFV 17D E protein coding sequence is replaced with the E protein coding sequence variant of a polynucleotide having SEQ ID NO:9, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:9, wherein the variant is not the YFV 17D sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO: 12 (YF-DW).

In various embodiments, the E protein of the deoptimized YFV is encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO:12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:12 (YF-DW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:12 (YF-DW).

In various embodiments, the deoptimized YFV is encoded by YFV 17D, wherein the YFV 17D E protein coding sequence is replaced with the E protein encoded by a variant of a polynucleotide having SEQ ID NO: 12 (YF-DW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO:12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:13 (YF-WD).

In various embodiments, the E protein of the deoptimized YFV is encoded by a variant of a polynucleotide having SEQ ID NO:13 (YF-DW), wherein the variant is not the YFV 17D sequence.

In particular embodiments, variant of a polynucleotide having SEQ ID NO:13 (YF-DW) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO:13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant of a polynucleotide having SEQ ID NO: 13 (YF-WD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:13 (YF-WD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:13 (YF-WD).

In various embodiments, the deoptimized YFV is encoded by YFV 17D, wherein the YFV 17D E protein coding sequence is replaced with the E protein encoded by a variant of a polynucleotide having SEQ ID NO: 13 (YF-WD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:13 (YF-WD), wherein the variant is not the YFV 17D sequence.

In particular embodiments, variant of a polynucleotide having SEQ ID NO:13 (YF-WD) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO:13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant SEQ ID NO: 13 (YF-WD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO: 14 (YF-DD).

In various embodiments, the E protein of the deoptimized YFV is encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO: 14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:14 (YF-DD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:14 (YF-DD).

In various embodiments, the deoptimized YFV is encoded by YFV 17D, wherein the YFV 17D E protein coding sequence is replaced with the E protein encoded by a variant of a polynucleotide having SEQ ID NO:14 (YF-DD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO:14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:15 (YF-DDDW).

In various embodiments, the E protein of the deoptimized YFV is encoded by a variant of a polynucleotide having SEQ ID NO:15 (YF-DDDW), wherein the variant is not the YFV 17D sequence.

In particular embodiments, variant of a polynucleotide having SEQ ID NO: 15 (YF-DDDW) has at least 95%, 96%, 97%, 98% or 99% sequence identity to SEQ ID NO:15. In various embodiments, the variant of a polynucleotide having SEQ ID NO:15 has up to 20 mutations in SEQ ID NO:15. In various embodiments, the variant of a polynucleotide having SEQ ID NO:15 has up to 10 mutations in SEQ ID NO: 15. In various embodiments, the variant of a polynucleotide having SEQ ID NO: 15 has up to 5 mutations in SEQ ID NO: 15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:15 (YF-DDDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:15 (YF-DDDW).

In various embodiments, the deoptimized YFV is encoded by YFV 17D, wherein the YFV 17D E protein coding sequence is replaced with the E protein encoded by a variant of a polynucleotide having SEQ ID NO:15 (YF-DDDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO: 15 (YF-DDDW), wherein the variant is not the YFV 17D sequence. In particular embodiments, variant of a polynucleotide having SEQ ID NO: 15 (YF-DDDW) has at least 95%, 96%, 97%, 98% or 99% sequence identity to SEQ ID NO: 15. In various embodiments, the variant of a polynucleotide having SEQ ID NO: 15 has up to 20 mutations in SEQ ID NO: 15. In various embodiments, the variant of a polynucleotide having SEQ ID NO: 15 has up to 10 mutations in SEQ ID NO: 15. In various embodiments, the variant of a polynucleotide having SEQ ID NO: 15 has up to 5 mutations in SEQ ID NO:15.

In particular embodiments, the variant of a polynucleotide having SEQ ID NO: 15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

Wild-type and Deoptimized Yellow Fever Full Length and
Envelope (E) Protein Coding Sequences

| | | SEQ |
|---|---|---|
| | | ID |
| | Sequence | NO: |
| YF full | AGTAAATCCTGTGTGCTAATTGAGGTGCATTGGTCTGCAAATCGAGTTGCTAGGCA | 1 |
| length wt | ATAAACACATTTGGATTAATTTTAATCGTTCGTTGAGCGATTAGCAGAGAACTGAC | |
| genome, | CAGAACATGTCTGGTCGTAAAGCTCAGGGAAAAACCCTGGGCGTCAATATGGTACG | |
| bold is in- | ACGAGGAGTTCGCTCCTTGTCAAACAAAATAAAACAAAAAACAAAACAAATTGGAA | |
| frame E | ACAGACCTGGACCTTCAAGAGGTGTTCAAGGATTTATCTTTTTCTTTTTGTTCAAC | |
| gene | ATTTTGACTGGAAAAAAGATCACAGCCCACCTAAAGAGGTTGTGGAAAATGCTGGA | |
| | CCCAAGACAAGGCTTGGCTGTTCTAAGGAAAGTCAAGAGAGTGGTGGCCAGTTTGA | |
| | TGAGAGGATTGTCCTCAAGGAAACGCCGTTCCCATGATGTTCTGACTGTGCAATTC | |
| | CTAATTTTGGGAATGCTGTTGATGACGGGTGGAGTGACCTTGGTGCGGAAAAACAG | |
| | ATGGTTGCTCCTAAATGTGACATCTGAGGACCTCGGGAAAACATTCTCTGTGGGCA | |
| | CAGGCAACTGCACAACAAACATTTTGGAAGCCAAGTACTGGTGCCCAGACTCAATG | |
| | GAATACAACTGTCCCAATCTCAGTCCAAGAGAGGAGCCAGATGACATTGATTGCTG | |
| | GTGCTATGGGGTGGAAAACGTTAGAGTCGCATATGGTAAGTGTGACTCAGCAGGCA | |
| | GGTCTAGGAGGTCAAGAAGGGCCATTGACTTGCCTACGCATGAAAACCATGGTTTG | |
| | AAGACCCGGCAAGAAAAATGGATGACTGGAAGAATGGGTGAAAGGCAACTCCAAAA | |
| | GATTGAGAGATGGTTCGTGAGGAACCCCTTTTTTGCAGTGACGGCTCTGACCATTG | |
| | CCTACCTTGTGGGAAGCAACATGACGCAACGAGTCGTGATTGCCCTACTGGTCTTG | |
| | GCTGTTGGTCCGGCCTACTCAGCTCACTGCATTGGAATTACTGACAGGGATTTCAT | |
| | TGAGGGGGTGCATGGAGGAACTTGGGTTTCAGCTACCCTGGAGCAAGACAAGTGTG | |
| | TCACTGTTATGGCCCCTGACAAGCCTTCATTGGACATCTCACTAGAGACAGTAGCC | |
| | ATTGATAGACCTGCTGAGGTGAGGAAAGTGTGTTACAATGCAGTTCTCACTCATGT | |
| | GAAGATTAATGACAAGTGCCCCAGCACTGGAGAGGCCCACCTAGCTGAAGAGAACG | |
| | AAGGGGACAATGCGTGCAAGCGCACTTATTCTGATAGAGGCTGGGGCAATGGCTGT | |
| | GGCCTATTTGGGAAAGGGAGCATTGTGGCATGCGCCAAATTCACTTGTGCCAAATC | |
| | CATGAGTTTGTTTGAGGTTGATCAGACCAAAATTCAGTATGTCATCAGAGCACAAT | |
| | TGCATGTAGGGGCCAAGCAGGAAAATTGGACTACCGACATTAAGACTCTCAAGTTT | |
| | GATGCCCTGTCAGGCTCCCAGGAAGTCGAGTTCATTGGGTATGGAAAAGCTACACT | |
| | GGAATGCCAGGTGCAAACTGCGGTGGACTTTGGTAACAGTTACATCGCTGAGATGG | |
| | AAACAGAGAGCTGGATAGTGGACAGACAGTGGGCCCAGGACTTGACCCTGCCATGG | |
| | CAGAGTGGAAGTGGCGGGGTGTGGAGAGAGATGCATCATCTTGTCGAATTTGAACC | |
| | TCCGCATGCCGCCACTATCAGAGTACTGGCCCTGGGAAACCAGGAAGGCTCCTTGA | |
| | AAACAGCTCTTACTGGCGCAATGAGGGTTACAAAGGACACAAATGACAACAACCTT | |
| | TACAAACTACATGGTGGACATGTTTCTTGCAGAGTGAAATTGTCAGCTTTGACACT | |
| | CAAGGGGACATCCTACAAAATATGCACTGACAAAATGTTTTTTGTCAAGAACCCAA | |
| | CTGACACTGGCCATGGCACTGTTGTGATGCAGGTGAAAGTGTCAAAAGGAGCCCCC | |
| | TGCAGGATTCCAGTGATAGTAGCTGATGATCTTACAGCGGCAATCAATAAAGGCAT | |
| | TTTGGTTACAGTTAACCCCATCGCCTCAACCAATGATGATGAAGTGCTGATTGAGG | |
| | TGAACCCACCTTTTGGAGACAGCTACATTATCGTTGGGAGAGGAGATTCACGTCTC | |
| | ACTTACCAGTGGCACAAAGAGGGAAGCTCAATAGGAAAGTTGTTCACTCAGACCAT | |
| | GAAAGGCGTGGAACGCCTGGCCGTCATGGGAGACACCGCCTGGGATTTCAGCTCCG | |
| | CTGGAGGGTTCTTCACTTCGGTTGGGAAAGGAATTCATACGGTGTTTGGCTCTGCC | |
| | TTTCAGGGGCTATTTGGCGGCTTGAACTGGATAACAAAGGTCATCATGGGGGCGGT | |
| | ACTTATATGGGTTGGCATCAACACAAGAAACATGACAATGTCCATGAGCATGATCT | |
| | TGGTAGGAGTGATCATGATGTTTTTGTCTCTAGGAGTTGGGGCGGATCAAGGATGC | |
| | GCCATCAACTTTGGCAAGAGAGAGCTCAAGTGCGGAGATGGTATCTTCATATTTAG | |
| | AGACTCTGATGACTGGCTGAACAAGTACTCATACTATCCAGAAGATCCTGTGAAGC | |
| | TTGCATCAATAGTGAAAGCCTCTTTTGAAGAAGGGAAGTGTGGCCTAAATTCAGTT | |
| | GACTCCCTTGAGCATGAGATGTGGAGAAGCAGGGCAGATGAGATCAATGCCATTTT | |
| | TGAGGAAAACGAGGTGGACATTTCTGTTGTCGTGCAGGATCCAAAGAATGTTTACC | |
| | AGAGAGGAACTCATCCATTTTCCAGAATTCGGGATGGTCTGCAGTATGGTTGGAAG | |
| | ACTTGGGGTAAGAACCTTGTGTTCTCCCCAGGGAGGAAGAATGGAAGCTTCATCAT | |
| | AGATGGAAAGTCCAGGAAAGAATGCCCGTTTTCAAACCGGGTCTGGAATTCTTTCC | |
| | AGATAGAGGAGTTTGGGACGGGAGTGTTCACCACACGCGTGTACATGGACGCAGTC | |
| | TTTGAATACACCATAGACTGCGATGGATCTATCTTGGGTGCAGCGGTGAACGGAAA | |
| | AAAGAGTGCCCATGGCTCTCCAACATTTTGGATGGGAAGTCATGAAGTAAATGGGA | |
| | CATGGATGATCCACACCTTGGAGGCATTAGATTACAAGGAGTGTGAGTGGCCACTG | |
| | ACACATACGATTGGAACATCAGTTGAAGAGAGTGAAATGTTCATGCCGAGATCAAT | |
| | CGGAGGCCCAGTTAGCTCTCACAATCATATCCCTGGATACAAGGTTCAGACGAACG | |
| | GACCTTGGATGCAGGTACCACTAGAAGTGAAGAGAGAAGCTTGCCCAGGGACTAGC | |
| | GTGATCATTGATGGCAACTGTGATGGACGGGGAAAATCAACCAGATCCACCACGGA | |
| | TAGCGGGAAAGTTATTCCTGAATGGTGTTGCCGCTCCTGCACAATGCCGCCTGTGA | |
| | GCTTCCATGGTAGTGATGGGTGTTGGTATCCCATGGAAATTAGGCCAAGGAAAACG | |
| | CATGAAAGCCATCTGGTGCGCTCCTGGGTTACAGCTGGAGAAATACATGCTGTCCC | |
| | TTTTGGTTTGGTGAGCATGATGATAGCAATGGAAGTGGTCCTAAGGAAAAGACAGG | |
| | GACCAAAGCAAATGTTGGTTGGAGGAGTAGTGCTCTTGGGAGCAATGCTGGTCGGG | |
| | CAAGTAACTCTCCTTGATTTGCTGAAACTCACAGTGGCTGTGGGATTGCATTTCCA | |
| | TGAGATGAACAATGGAGGAGACGCCATGTATATGGCGTTGATTGCTGCCTTTTCAA | |
| | TCAGACCAGGGCTGCTCATCGGCTTTGGGCTCAGGACCCTATGGAGCCCTCGGGAA | |
| | CGCCTTGTGCTGACCCTAGGAGCAGCCATGGTGGAGATTGCCTTGGGTGGCGTGAT | |
| | GGGCGGCCTGTGGAAGTATCTAAATGCAGTTTCTCTCTGCATCCTGACAATAAATG | |
| | CTGTTGCTTCTAGGAAAGCATCAAATACCATCTTGCCCCTCATGGCTCTGTTGACA | |
| | CCTGTCACTATGGCTGAGGTGAGACTTGCCGCAATGTTCTTTTGTGCCGTGGTTAT | |
| | CATAGGGGTCCTTCACCAGAATTTCAAGGACACCTCCATGCAGAAGACTATACCTC | |
| | TGGTGGCCCTCACACTCACATCTTACCTGGGCTTGACACAACCTTTTTTGGGCCTG | |
| | TGTGCATTTCTGGCAACCCGCATATTTGGGCGAAGGAGTATCCCAGTGAATGAGGC | |
| | ACTCGCAGCAGCTGGTCTAGTGGGAGTGCTGGCAGGACTGGCTTTTCAGGAGATGG | |
| | AGAACTTCCTTGGTCCGATTGCAGTTGGAGGACTCCTGATGATGCTGGTTAGCGTG | |
| | GCTGGGAGGGTGGATGGGCTAGAGCTCAAGAAGCTTGGTGAAGTTTCATGGGAAGA | |
| | GGAGGCGGAGATCAGCGGGAGTTCCGCCCGCTATGATGTGGCACTCAGTGAACAAG | |
| | GGGAGTTCAAGCTGCTTTCTGAAGAGAAAGTGCCATGGGACCAGGTTGTGATGACC | |
| | TCGCTGGCCTTGGTTGGGGCTGCCCTCCATCCATTTGCTCTTCTGCTGGTCCTTGC | |
| | TGGGTGGCTGTTTCATGTCAGGGGAGCTAGGAGAAGTGGGGATGTCTTGTGGGATA | |
| | TTCCCACTCCTAAGATCATCGAGGAATGTGAACATCTGGAGGATGGGATTTATGGC | |
| | ATATTCCAGTCAACCTTCTTGGGGGCCTCCCAGCGAGGAGTGGGAGTGGCACAGGG | |
| | AGGGGTGTTCCACACAATGTGGCATGTCACAAGAGGAGCTTTCCTTGTCAGGAATG | |
| | GCAAGAAGTTGATTCCATCTTGGGCTTCAGTAAAGGAAGACCTTGTCGCCTATGGT | |
| | GGCTCATGGAAGTTGGAAGGCAGATGGGATGGAGAGGAAGAGGTCCAGTTGATCGC | |
| | GGCTGTTCCAGGAAAGAACGTGGTCAACGTCCAGACAAAACCGAGCTTGTTCAAAG | |
| | TGAGGAATGGGGGAGAAATCGGGGCTGTCGCTCTTGACTATCCGAGTGGCACTTCA | |
| | GGATCTCCTATTGTTAACAGGAACGGAGAGGTGATTGGGCTGTACGGCAATGGCAT | |
| | CCTTGTCGGTGACAACTCCTTCGTGTCCGCCATATCCCAGACTGAGGTGAAGGAAG | |
| | AAGGAAAGGAGGAGCTCCAAGAGATCCCGACAATGCTAAAGAAAGGAATGACAACT | |
| | GTCCTTGATTTTCATCCTGGAGCTGGGAAGACAAGACGTTTCCTCCCACAGATCTT | |
| | GGCCGAGTGCGCACGGAGACGCTTGCGCACTCTTGTGTTGGCCCCCACCAGGGTTG | |
| | TTCTTTCTGAAATGAAGGAGGCTTTTCACGGCCTGGACGTGAAATTCCACACACAG | |
| | GCTTTTTCCGCTCACGGCAGCGGGAGAGAAGTCATTGATGCTATGTGCCATGCCAC | |
| | CCTAACTTACAGGATGTTGGAACCAACTAGGGTTGTTAACTGGGAAGTGATCATTA | |
| | TGGATGAAGCCCATTTTTTGGATCCAGCTAGCATAGCCGCTAGAGGTTGGGCAGCG | |
| | CACAGAGCTAGGGCAAATGAAAGTGCAACAATCTTGATGACAGCCACACCGCCTGG | |
| | GACTAGTGATGAATTTCCACATTCAAATGGTGAAATAGAAGATGTTCAAACGGACA | |
| | TACCCAGTGAGCCCTGGAACACAGGGCATGACTGGATCCTGGCTGACAAAAGGCCC | |
| | ACGGCATGGTTCCTTCCATCCATCAGAGCTGCAAATGTCATGGCTGCCTCTTTGCG | |
| | TAAGGCTGGAAAGAGTGTGGTGGTCCTGAACAGGAAAACCTTTGAGAGAGAATACC | |
| | CCACGATAAAGCAGAAGAAACCTGACTTTATATTGGCCACTGACATAGCTGAAATG | |
| | GGAGCCAACCTTTGCGTGGAGCGAGTGCTGGATTGCAGGACGGCTTTTAAGCCTGT | |
| | GCTTGTGGATGAAGGGAGGAAGGTGGCAATAAAAGGGCCACTTCGTATCTCCGCAT | |
| | CCTCTGCTGCTCAAAGGAGGGGGCGCATTGGGAGAAATCCCAACAGAGATGGAGAC | |
| | TCATACTACTATTCTGAGCCTACAAGTGAAAATAATGCCCACCACGTCTGCTGGTT | |
| | GGAGGCCTCAATGCTCTTGGACAACATGGAGGTGAGGGGTGGAATGGTCGCCCCAC | |
| | TCTATGGCGTTGAAGGAACTAAAACACCAGTTTCCCCTGGTGAAATGAGACTGAGG | |
| | GATGACCAGAGGAAAGTCTTCAGAGAACTAGTGAGGAATTGTGACCTGCCCGTTTG | |
| | GCTTTCGTGGCAAGTGGCCAAGGCTGGTTTGAAGACGAATGATCGTAAGTGGTGTT | |
| | TTGAAGGCCCTGAGGAACATGAGATCTTGAATGACAGCGGTGAAACAGTGAAGTGC | |
| | AGGGCTCCTGGAGGAGCAAAGAAGCCTCTGCGCCCAAGGTGGTGTGATGAAAGGGT | |
| | GTCATCTGACCAGAGTGCGCTGTCTGAATTTATTAAGTTTGCTGAAGGTAGGAGGG | |
| | GAGCTGCTGAAGTGCTAGTTGTGCTGAGTGAACTCCCTGATTTCCTGGCTAAAAAA | |
| | GGTGGAGAGGCAATGGATACCATCAGTGTGTTTCTCCACTCTGAGGAAGGCTCTAG | |
| | GGCTTACCGCAATGCACTATCAATGATGCCTGAGGCAATGACAATAGTCATGCTGT | |
| | TTATACTGGCTGGACTACTGACATCGGGAATGGTCATCTTTTTCATGTCTCCCAAA | |
| | GGCATCAGTAGAATGTCTATGGCGATGGGCACAATGGCCGGCTGTGGATATCTCAT | |
| | GTTCCTTGGAGGCGTCAAACCCACTCACATCTCCTATATCATGCTCATATTCTTTG | |
| | TCCTGATGGTGGTTGTGATCCCCGAGCCAGGGCAACAAAGGTCCATCCAAGACAAC | |
| | CAAGTGGCATACCTCATTATTGGCATCCTGACGCTGGTTTCAGCGGTGGCAGCCAA | |
| | CGAGCTAGGCATGCTGGAGAAAACCAAAGAGGACCTCTTTGGGAAGAAGAACTTAA | |
| | TTCCATCTAGTGCTTCACCCTGGAGTTGGCCGGATCTTGACCTGAAGCCAGGAGCT | |
| | GCCTGGACAGTGTACGTTGGCATTGTTACAATGCTCTCTCCAATGTTGCACCACTG | |
| | GATCAAAGTCGAATATGGCAACCTGTCTCTGTCTGGAATAGCCCAGTCAGCCTCAG | |
| | TCCTTTCTTTCATGGACAAGGGGATACCATTCATGAAGATGAATATCTCGGTCATA | |
| | ATGCTGCTGGTCAGTGGCTGGAATTCAATAACAGTGATGCCTCTGCTCTGTGGCAT | |
| | AGGGTGCGCCATGCTCCACTGGTCTCTCATTTTACCTGGAATCAAAGCGCAGCAGT | |
| | CAAAGCTTGCACAGAGAAGGGTGTTCCATGGCGTTGCCAAGAACCCTGTGGTTGAT | |
| | GGGAATCCAACAGTTGACATTGAGGAAGCTCCTGAAATGCCTGCCCTTTATGAGAA | |
| | GAAACTGGCTCTATATCTCCTTCTTGCTCTCAGCCTAGCTTCTGTTGCCATGTGCA | |
| | GAACGCCCTTTTCATTGGCTGAAGGCATTGTCCTAGCATCAGCTGCCCTAGGGCCG | |
| | CTCATAGAGGGAAACACCAGCCTTCTTTGGAATGGACCCATGGCTGTCTCCATGAC | |
| | AGGAGTCATGAGGGGGAATCACTATGCTTTTGTGGGAGTCATGTACAATCTATGGA | |
| | AGATGAAAACTGGACGCCGGGGGAGCGCGAATGGAAAAACTTTGGGTGAAGTCTGG | |
| | AAGAGGGAACTGAATCTGTTGGACAAGCGACAGTTTGAGTTGTATAAAAGGACCGA | |
| | CATTGTGGAGGTGGATCGTGATACGGCACGCAGGCATTTGGCCGAAGGGAAGGTGG | |
| | ACACCGGGGTGGCGGTCTCCAGGGGGACCGCAAAGTTAAGGTGGTTCCATGAGCGT | |
| | GGCTATGTCAAGCTGGAAGGTAGGGTGATTGACCTGGGGTGTGGCCGCGGAGGCTG | |
| | GTGTTACTACGCTGCTGCGCAAAAGGAAGTGAGTGGGGTCAAAGGATTTACTCTTG | |
| | GAAGAGACGGCCATGAGAAACCCATGAATGTGCAAAGTCTGGGATGGAACATCATC | |
| | ACCTTCAAGGACAAAACTGATATCCACCGCCTAGAACCAGTGAAATGTGACACCCT | |
| | TTTGTGTGACATTGGAGAGTCATCATCGTCATCGGTCACAGAGGGGGAAAGGACCG | |
| | TGAGAGTTCTTGATACTGTAGAAAAATGGCTGGCTTGTGGGGTTGACAACTTCTGT | |
| | GTGAAGGTGTTAGCTCCATACATGCCAGATGTTCTCGAGAAACTGGAATTGCTCCA | |
| | AAGGAGGTTTGGCGGAACAGTGATCAGGAACCCTCTCTCCAGGAATTCCACTCATG | |
| | AAATGTACTACGTGTCTGGAGCCCGCAGCAATGTCACATTTACTGTGAACCAAACA | |
| | TCCCGCCTCCTGATGAGGAGAATGAGGCGTCCAACTGGAAAAGTGACCCTGGAGGC | |
| | TGACGTCATCCTCCCAATTGGGACACGCAGTGTTGAGACAGACAAGGGACCCCTGG | |
| | ACAAAGAGGCCATAGAAGAAAGGGTTGAGAGGATAAAATCTGAGTACATGACCTCT | |
| | TGGTTTTATGACAATGACAACCCCTACAGGACCTGGCACTACTGTGGCTCCTATGT | |
| | CACAAAAACCTCAGGAAGTGCGGCGAGCATGGTAAATGGTGTTATTAAAATTCTGA | |
| | CATATCCATGGGACAGGATAGAGGAGGTCACAAGAATGGCAATGACTGACACAACC | |
| | CCTTTTGGACAGCAAAGAGTGTTTAAAGAAAAAGTTGACACCAGAGCAAAGGATCC | |
| | ACCAGCGGGAACTAGGAAGATCATGAAAGTTGTCAACAGGTGGCTGTTCCGCCACC | |
| | TGGCCAGAGAAAAGAACCCCAGACTGTGCACAAAGGAAGAATTTATTGCAAAAGTC | |
| | CGAAGTCATGCAGCCATTGGAGCTTACCTGGAAGAACAAGAACAGTGGAAGACTGC | |
| | CAATGAGGCTGTCCAAGACCCAAAGTTCTGGGAACTGGTGGATGAAGAAAGGAAGC | |
| | TGCACCAACAAGGCAGGTGTCGGACTTGTGTGTACAACATGATGGGGAAAAGAGAG | |
| | AAGAAGCTGTCAGAGTTTGGGAAAGCAAAGGGAAGCCGTGCCATATGGTATATGTG | |
| | GCTGGGAGCGCGGTATCTTGAGTTTGAGGCCCTGGGATTCCTGAATGAGGACCATT | |
| | GGGCTTCCAGGGAAAACTCAGGAGGAGGAGTGGAAGGCATTGGCTTACAATACCTA | |
| | GGATATGTGATCAGAGACCTGGCTGCAATGGATGGTGGTGGATTCTACGCGGATGA | |
| | CACCGCTGGATGGGACACGCGCATCACAGAGGCAGACCTTGATGATGAACAGGAGA | |
| | TCTTGAACTACATGAGCCCACATCACAAAAAACTGGCACAAGCAGTGATGGAAATG | |
| | ACATACAAGAACAAAGTGGTGAAAGTGTTGAGACCAGCCCCAGGAGGGAAAGCCTA | |
| | CATGGATGTCATAAGTCGACGAGACCAGAGAGGATCCGGGCAGGTAGTGACTTATG | |
| | CTCTGAACACCATCACCAACTTGAAAGTCCAATTGATCAGAATGGCAGAAGCAGAG | |
| | ATGGTGATACATCACCAACATGTTCAAGATTGTGATGAATCAGTTCTGACCAGGCT | |
| | GGAGGCATGGCTCACTGAGCACGGATGTAACAGACTGAAGAGGATGGCGGTGAGTG | |
| | GAGACGACTGTGTGGTCCGGCCCATCGATGACAGGTTCGGCCTGGCCCTGTCCCAT | |
| | CTCAACGCCATGTCCAAGGTTAGAAAGGACATATCTGAATGGCAGCCATCAAAAGG | |
| | GTGGAATGATTGGGAGAATGTGCCCTTCTGTTCCCACCACTTCCATGAACTACAGC | |
| | TGAAGGATGGCAGGAGGATTGTGGTGCCTTGCCGAGAACAGGACGAGCTCATTGGG | |
| | AGAGGAAGGGTGTCTCCAGGAAACGGCTGGATGATCAAGGAAACAGCTTGCCTCAG | |
| | CAAAGCCTATGCCAACATGTGGTCACTGATGTATTTTCACAAAAGGGACATGAGGC | |
| | TACTGTCATTGGCTGTTTCCTCAGCTGTTCCCACCTCATGGGTTCCACAAGGACGC | |
| | ACAACATGGTCGATTCATGGGAAAGGGGAGTGGATGACCACGGAAGACATGCTTGA | |
| | GGTGTGGAACAGAGTATGGATAACCAACAACCCACACATGCAGGACAAGACAATGG | |
| | TGAAAAAATGGAGAGATGTCCCTTATCTAACCAAGAGACAAGACAAGCTGTGCGGA | |
| | TCACTGATTGGAATGACCAATAGGGCCACCTGGGCCTCCCACATCCATTTGGTCAT | |
| | CCATCGTATCCGAACGCTGATTGGACAGGAGAAATACACTGACTACCTAACAGTCA | |
| | TGGACAGGTATTCTGTGGATGCTGACCTGCAACTGGGTGAGCTTATCTGAAACACC | |
| | ATCTAACAGGAATAACCGGGATACAAACCACGGGTGGAGAACCGGACTCCCCACAA | |
| | CCTGAAACCGGGATATAAACCACGGCTGGAGAACCGGACTCCGCACTTAAAATGAA | |
| | ACAGAAACCGGGATAAAAACTACGGATGGAGAACCGGACTCCACACATTGAGACAG | |
| | AAGAAGTTGTCAGCCCAGAACCCCACACGAGTTTTGCCACTGCTAAGCTGTGAGGC | |
| | AGTGCAGGCTGGGACAGCCGACCTCCAGGTTGCGAAAAACCTGGTTTCTGGGACCT | |
| | CCCACCCCAGAGTAAAAAGAACGGAGCCTCCGCTACCACCCTCCCACGTGGTGGTA | |
| | GAAAGACGGGGTCTAGAGGTTAGAGGAGACCCTCCAGGGAACAAATAGTGGGACCA | |
| | TATTGACGCCAGGGAAAGACCGGAGTGGTTCTCTGCTTTTCCTCCAGAGGTCTGTG | |
| | AGCACAGTTTGCTCAAGAATAAGCAGACCTTTGGATGACAAACACAAAACCACT | |
| YF wt E- | GCTCACTGCATTGGAATTACTGACAGGGATTTCATTGAGGGGGTGCATGGAGGAAC | 2 |
| gene (1479 | TTGGGTTTCAGCTACCCTGGAGCAAGACAAGTGTGTCACTGTTATGGCCCCTGACA | |
| nts) | AGCCTTCATTGGACATCTCACTAGAGACAGTAGCCATTGATAGACCTGCTGAGGTG | |
| | AGGAAAGTGTGTTACAATGCAGTTCTCACTCATGTGAAGATTAATGACAAGTGCCC | |
| | CAGCACTGGAGAGGCCCACCTAGCTGAAGAGAACGAAGGGGACAATGCGTGCAAGC | |
| | GCACTTATTCTGATAGAGGCTGGGGCAATGGCTGTGGCCTATTTGGGAAAGGGAGC | |
| | ATTGTGGCATGCGCCAAATTCACTTGTGCCAAATCCATGAGTTTGTTTGAGGTTGA | |
| | TCAGACCAAAATTCAGTATGTCATCAGAGCACAATTGCATGTAGGGGCCAAGCAGG | |
| | AAAATTGGACTACCGACATTAAGACTCTCAAGTTTGATGCCCTGTCAGGCTCCCAG | |
| | GAAGTCGAGTTCATTGGGTATGGAAAAGCTACACTGGAATGCCAGGTGCAAACTGC | |
| | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCTTGAAAACAGCTCTTACTGGCGCAA | |
| | TGAGGGTTACAAAGGACACAAATGACAACAACCTTTACAAACTACATGGTGGACAT | |
| | GTTTCTTGCAGAGTGAAATTGTCAGCTTTGACACTCAAGGGGACATCCTACAAAAT | |
| | ATGCACTGACAAAATGTTTTTTGTCAAGAACCCAACTGACACTGGCCATGGCACTG | |
| | TTGTGATGCAGGTGAAAGTGTCAAAAGGAGCCCCCTGCAGGATTCCAGTGATAGTA | |
| | GCTGATGATCTTACAGCGGCAATCAATAAAGGCATTTTGGTTACAGTTAACCCCAT | |
| | CGCCTCAACCAATGATGATGAAGTGCTGATTGAGGTGAACCCACCTTTTGGAGACA | |
| | GCTACATTATCGTTGGGAGAGGAGATTCACGTCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGCGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCG | |
| YF | GCTCACTGCATTGGAATTACTGACAGGGATTTCATTGAGGGGGTGCATGGAGGAAC | 3 |
| E-gene | TTGGGTTTCAGCTACCCTGGAGCAAGACAAGTGTGTCACTGTTATGGCCCCTGACA | |
| WD (1479 | AGCCTTCATTGGACATCTCACTAGAGACAGTAGCCATTGATAGACCTGCTGAGGTG | |
| nts, lower- | AGGAAAGTGTGTTACAATGCAGTTCTCACTCATGTGAAGATTAATGACAAGTGCCC | |
| case | CAGCACTGGAGAGGCCCACCTAGCTGAAGAGAACGAAGGGGACAATGCGTGCAAGC | |
| represents | GCACTTATTCTGATAGAGGCTGGGGCAATGGCTGTGGCCTATTTGGGAAAGGGAGC | |
| CPD | ATTGTGGCATGCGCCAAATTCACTTGTGCCAAATCCATGAGTTTGTTTGAGGTTGA | |
| region) | TCAGACCAAAATTCAGTATGTCATCAGAGCACAATTGCATGTAGGGGCCAAGCAGG | |
| | AAAATTGGACTACCGACATTAAGACTCTCAAGTTTGATGCCCTGTCAGGCTCCCAG | |
| | GAAGTCGAGTTCATTGGGTATGGAAAAGCTACACTGGAATGCCAGGTGCAAACTGC | |
| | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCcttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagactgacataccaatggcataaagag | |
| | ggatcgtcaatcggtaagttgtttacacagactatgaaaggggtggagagattggc | |
| | cgttatgggcgataccgcttgggactttagttccgccggagggttttttactagcg | |
| | tcggaaaggggatacataccgtattcggatccgcttttcaggggttgttcggcgga | |
| | ctgaattggattacgaaagtgattatgggcgccgtacttatttgggtggggattaa | |
| | cactaggaatatgactatgtctatgtctatgatactagtcggagtgattatgatgt | |
| | ttctgtcattgggcgtaggcgct | |
| YF | GCTCACTGCATTGGAATTACTGACAGGGATtttatcgagggggtgcatggcggaac | 4 |
| E-gene | ttgggttagcgctacactcgaacaggacaaatgcgttaccgttatggcccccgata | |
| DW (1479 | agcctagcctagacattagtctcgaaaccgttgcgatcgatagacccgccgaagtg | |
| nts, lower- | agaaaagtgtgttataacgccgtactgactcacgttaagattaacgacaaatgccc | |
| case | tagtacaggcgaagcgcatctagccgaagagaacgagggcgataacgcatgcaaac | |
| represents | gtacttatagcgatagggggtgggggaacggatgcggattgttcggtaaggggtca | |
| CPD | atcgtcgcatgcgctaagtttacatgcgctaagtctatgtcattgttcgaagtcga | |
| region) | tcagactaagattcagtacgtgattagagcgcaattgcatgtgggagcgaaacagg | |
| | agaattggactactgacattaagacactgaaattcgacgcccttagcggatcacag | |
| | gaggtcgagtttattgggtacggaaaagcgacactcgagtgtcaggtgcagactgc | |
| | cgttgactttggcaattcatacatagccgaaatggagacagagtcatggatcgttg | |
| | acagacagtgggcccaggatctgacattgccatggcaatccggatccggaggcgtt | |
| | tggcgcgaaatgcatcatctagtcgagttcgaaccgccacatgccgctacaatcag | |
| | agtgttggccctaggcaatcaggagggaTCCTTGAAAACAGCTCTTACTGGCGCAA | |
| | TGAGGGTTACAAAGGACACAAATGACAACAACCTTTACAAACTACATGGTGGACAT | |
| | GTTTCTTGCAGAGTGAAATTGTCAGCTTTGACACTCAAGGGGACATCCTACAAAAT | |
| | ATGCACTGACAAAATGTTTTTTGTCAAGAACCCAACTGACACTGGCCATGGCACTG | |
| | TTGTGATGCAGGTGAAAGTGTCAAAAGGAGCCCCCTGCAGGATTCCAGTGATAGTA | |
| | GCTGATGATCTTACAGCGGCAATCAATAAAGGCATTTTGGTTACAGTTAACCCCAT | |
| | CGCCTCAACCAATGATGATGAAGTGCTGATTGAGGTGAACCCACCTTTTGGAGACA | |
| | GCTACATTATCGTTGGGAGAGGAGATTCACGTCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGCGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCG | |
| YF | GCTCACTGCATTGGAATTACTGACAGGGATtttatcgagggggtgcatggcggaac | 5 |
| E-gene | ttgggttagcgctacactcgaacaggacaaatgcgttaccgttatggcccccgata | |
| DD (1479 | agcctagcctagacattagtctcgaaaccgttgcgatcgatagacccgccgaagtg | |
| nts, lower- | agaaaagtgtgttataacgccgtactgactcacgttaagattaacgacaaatgccc | |
| case | tagtacaggcgaagcgcatctagccgaagagaacgagggcgataacgcatgcaaac | |
| represents | gtacttatagcgatagggggtgggggaacggatgcggattgttcggtaaggggtca | |
| CPD | atcgtcgcatgcgctaagtttacatgcgctaagtctatgtcattgttcgaagtcga | |
| region) | tcagactaagattcagtacgtgattagagcgcaattgcatgtgggagcgaaacagg | |
| | agaattggactactgacattaagacactgaaattcgacgcccttagcggatcacag | |
| | gaggtcgagtttattgggtacggaaaagcgacactcgagtgtcaggtgcagactgc | |
| | cgttgactttggcaattcatacatagccgaaatggagacagagtcatggatcgttg | |
| | acagacagtgggcccaggatctgacattgccatggcaatccggatccggaggcgtt | |
| | tggcgcgaaatgcatcatctagtcgagttcgaaccgccacatgccgctacaatcag | |
| | agtgttggccctaggcaatcaggagggatcccttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagactgacataccaatggcataaagag | |
| | ggatcgtcaatcggtaagttgtttacacagactatgaaaggggtggagagattggc | |
| | cgttatgggcgataccgcttgggactttagttccgccggagggttttttactagcg | |
| | tcggaaaggggatacataccgtattcggatccgcttttcaggggttgttcggcgga | |
| | ctgaattggattacgaaagtgattatgggcgccgtacttatttgggtggggattaa | |
| | cactaggaatatgactatgtctatgtctatgatactagtcggagtgattatgatgt | |
| | ttctgtcattgggcgtaggcgct | |
| YF | GCTCACTGCATTGGAATTACTGACAGGGATtttatcgagggggtgcatggcggaac | 6 |
| E-gene | ttgggttagcgctacactcgaacaggacaaatgcgttaccgttatggcccccgata | |
| DDDW | agcctagcctagacattagtctcgaaaccgttgcgatcgatagacccgccgaagtg | |
| (1479 nts, | agaaaagtgtgttataacgccgtactgactcacgttaagattaacgacaaatgccc | |
| lower-case | tagtacaggcgaagcgcatctagccgaagagaacgagggcgataacgcatgcaaac | |
| represents | gtacttatagcgatagggggtgggggaacggatgcggattgttcggtaaggggtca | |
| CPD | atcgtcgcatgcgctaagtttacatgcgctaagtctatgtcattgttcgaagtcga | |
| region) | tcagactaagattcagtacgtgattagagcgcaattgcatgtgggagcgaaacagg | |
| | agaattggactactgacattaagacactgaaattcgacgcccttagcggatcacag | |
| | gaggtcgagtttattgggtacggaaaagcgacactcgagtgtcaggtgcagactgc | |
| | cgttgactttggcaattcatacatagccgaaatggagacagagtcatggatcgttg | |
| | acagacagtgggcccaggatctgacattgccatggcaatccggatccggaggcgtt | |
| | tggcgcgaaatgcatcatctagtcgagttcgaaccgccacatgccgctacaatcag | |
| | agtgttggccctaggcaatcaggagggatcccttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagaCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGGGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCG | |
| YF | GCTCACTGCATTGGAATTACTGACAGGGATTTCATTGAGGGGGTGCATGGAGGAAC | 7 |
| E-gene | TTGGGTTTCAGCTACCCTGGAGCAAGACAAGTGTGTCACTGTTATGGCCCCTGACA | |
| WWDW | AGCCTTCATTGGACATCTCACTAGAGACAGTAGCCATTGATAGACCTGCTGAGGTG | |
| (1479 nts, | AGGAAAGTGTGTTACAATGCAGTTCTCACTCATGTGAAGATTAATGACAAGTGCCC | |
| lower-case | CAGCACTGGAGAGGCCCACCTAGCTGAAGAGAACGAAGGGGACAATGCGTGCAAGC | |
| represents | GCACTTATTCTGATAGAGGCTGGGGCAATGGCTGTGGCCTATTTGGGAAAGGGAGC | |
| CPD | ATTGTGGCATGCGCCAAATTCACTTGTGCCAAATCCATGAGTTTGTTTGAGGTTGA | |
| region) | TCAGACCAAAATTCAGTATGTCATCAGAGCACAATTGCATGTAGGGGCCAAGCAGG | |
| | AAAATTGGACTACCGACATTAAGACTCTCAAGTTTGATGCCCTGTCAGGCTCCCAG | |
| | GAAGTCGAGTTCATTGGGTATGGAAAAGCTACACTGGAATGCCAGGTGCAAACTGC | |
| | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCcttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagaCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGCGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCG | |
| E-gene of | GCTCACTGCATTGGAATTACTGACAGGGATTTCATTGAGGGGGTGCATGGAGGAAC | 8 |
| YF-17D | TTGGGTTTCAGCTACCCTGGAGCAAGACAAGTGTGTCACTGTTATGGCCCCTGACA | |
| WD-E- | AGCCTTCATTGGACATCTCACTAGAGACAGTAGCCATTGATAGACCTGCTGAGGTG | |
| 153N (E- | AGGAAAGTGTGTTACAATGCAGTTCTCACTCATGTGAAGATTAATGACAAGTGCCC | |
| 153N | CAGCACTGGAGAGGCCCACCTAGCTGAAGAGAACGAAGGGGACAATGCGTGCAAGC | |
| codon is in | GCACTTATTCTGATAGAGGCTGGGGCAATGGCTGTGGCCTATTTGGGAAAGGGAGC | |
| bold, | ATTGTGGCATGCGCCAAATTCACTTGTGCCAAATCCATGAGTTTGTTTGAGGTTGA | |
| lower-case | TCAGACCAAAATTCAGTATGTCATCAGAGCACAATTGCATGTAGGGGCCAAGCAGG | |
| represents | AAAATTGGAATACCGACATTAAGACTCTCAAGTTTGATGCCCTGTCAGGCTCCCAG | |
| CPD | GAAGTCGAGTTCATTGGGTATGGAAAAGCTACACTGGAATGCCAGGTGCAAACTGC | |
| region) | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCcttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagactgacataccaatggcataaagag | |
| | ggatcgtcaatcggtaagttgtttacacagactatgaaaggggtggagagattggc | |
| | cgttatgggcgataccgcttgggactttagttccgccggagggttttttactagcg | |
| | tcggaaaggggatacataccgtattcggatccgcttttcaggggttgttcggcgga | |
| | ctgaattggattacgaaagtgattatgggcgccgtacttatttgggtggggattaa | |
| | cactaggaatatgactatgtctatgtctatgatactagtcggagtgattatgatgt | |
| | ttctgtcattgggcgtaggcgct | |
| E-gene of | GCTCACTGCATTGGAATTACTGACAGGGATTTCATTGAGGGGGTGCATGGAGGAAC | 9 |
| YF-17D | TTGGGTTTCAGCTACCCTGGAGCAAGACAAGTGTGTCACTGTTATGGCCCCTGACA | |
| WWDW- | AGCCTTCATTGGACATCTCACTAGAGACAGTAGCCATTGATAGACCTGCTGAGGTG | |
| E-153N (E- | AGGAAAGTGTGTTACAATGCAGTTCTCACTCATGTGAAGATTAATGACAAGTGCCC | |
| 153N | CAGCACTGGAGAGGCCCACCTAGCTGAAGAGAACGAAGGGGACAATGCGTGCAAGC | |
| codon is in | GCACTTATTCTGATAGAGGCTGGGGCAATGGCTGTGGCCTATTTGGGAAAGGGAGC | |
| bold, | ATTGTGGCATGCGCCAAATTCACTTGTGCCAAATCCATGAGTTTGTTTGAGGTTGA | |
| lower-case | TCAGACCAAAATTCAGTATGTCATCAGAGCACAATTGCATGTAGGGGCCAAGCAGG | |
| represents | AAAATTGGAATACCGACATTAAGACTCTCAAGTTTGATGCCCTGTCAGGCTCCCAG | |
| CPD | GAAGTCGAGTTCATTGGGTATGGAAAAGCTACACTGGAATGCCAGGTGCAAACTGC | |
| region) | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCcttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagaCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGCGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCG | |
| YF Wt | AHCIGITDRDFIEGVHGGTWVSATLEQDKCVTVMAPDKPSLDISLETVAIDRPAEV | 10 |
| E-protein | RKVCYNAVLTHVKINDKCPSTGEAHLAEENEGDNACKRTYSDRGWGNGCGLFGKGS | |
| sequences. | IVACAKFTCAKSMSLFEVDQTKIQYVIRAQLHVGAKQENWTTDIKTLKEDALSGSQ | |
| 493 AAs, | EVEFIGYGKATLECQVQTAVDEGNSYIAEMETESWIVDRQWAQDLTLPWQSGSGGV | |
| E153=T is | WREMHHLVEFEPPHAATIRVLALGNQEGSLKTALTGAMRVTKDTNDNNLYKLHGGH | |
| bolded. | VSCRVKLSALTLKGTSYKICTDKMFFVKNPTDTGHGTVVMQVKVSKGAPCRIPVIV | |
| E-protein | ADDLTAAINKGILVTVNPIASTNDDEVLIEVNPPFGDSYIIVGRGDSRLTYQWHKE | |
| sequences | GSSIGKLFTQTMKGVERLAVMGDTAWDESSAGGFFTSVGKGIHTVFGSAFQGLEGG | |
| of E-WD, | LNWITKVIMGAVLIWVGINTRNMTMSMSMILVGVIMMELSLGVGA | |
| E-DW, E- | | |
| DD, E- | | |
| WWDW, | | |
| and E- | | |
| DDDW are | | |
| the same as | | |
| this | | |
| sequence. | | |
| YF-17D | AHCIGITDRDFIEGVHGGTWVSATLEQDKCVTVMAPDKPSLDISLETVAIDRPAEV | 11 |
| WD-E- | RKVCYNAVLTHVKINDKCPSTGEAHLAEENEGDNACKRTYSDRGWGNGCGLFGKGS | |
| 153N | IVACAKFTCAKSMSLFEVDQTKIQYVIRAQLHVGAKQENWNTDIKTLKEDALSGSQ | |
| and | EVEFIGYGKATLECQVQTAVDEGNSYIAEMETESWIVDRQWAQDLTLPWQSGSGGV | |
| YF-17D | WREMHHLVEFEPPHAATIRVLALGNQEGSLKTALTGAMRVTKDTNDNNLYKLHGGH | |
| WWDW- | VSCRVKLSALTLKGTSYKICTDKMFFVKNPTDTGHGTVVMQVKVSKGAPCRIPVIV | |
| E-153N | ADDLTAAINKGILVTVNPIASTNDDEVLIEVNPPFGDSYIIVGRGDSRLTYQWHKE | |
| E-protein | GSSIGKLFTQTMKGVERLAVMGDTAWDESSAGGFFTSVGKGIHTVFGSAFQGLEGG | |
| sequences. | LNWITKVIMGAVLIWVGINTRNMTMSMSMILVGVIMMFLSLGVGA | |
| 493 AAs, | | |
| E153=N is | | |
| bolded. | | |
| YF-Env- | ATGACTGGAAGAATGGGTGAAAGGCAACTCCAAAAGATTGAGAGATGGTTCGTGAG | 12 |
| DW | GAACCCCTTTTTTGCAGTGACGGCTCTGACCATTGCCTACCTTGTGGGAAGCAACA | |
| | TGACGCAACGAGTCGTGATTGCCCTACTGGTCTTGGCTGTTGGTCCGGCCTACTCA | |
| | GCTCACTGCATTGGAATTACTGACAGGGATtttatcgagggggtgcatggcggaac | |
| | ttgggttagcgctacactcgaacaggacaaatgcgttaccgttatggcccccgata | |
| | agcctagcctagacattagtctcgaaaccgttgcgatcgatagacccgccgaagtg | |
| | agaaaagtgtgttataacgccgtactgactcacgttaagattaacgacaaatgccc | |
| | tagtacaggcgaagcgcatctagccgaagagaacgagggcgataacgcatgcaaac | |
| | gtacttatagcgatagggggtgggggaacggatgcggattgttcggtaaggggtca | |
| | atcgtcgcatgcgctaagtttacatgcgctaagtctatgtcattgttcgaagtcga | |
| | tcagactaagattcagtacgtgattagagcgcaattgcatgtgggagcgaaacagg | |
| | agaattggactactgacattaagacactgaaattcgacgcccttagcggatcacag | |
| | gaggtcgagtttattgggtacggaaaagcgacactcgagtgtcaggtgcagactgc | |
| | cgttgactttggcaattcatacatagccgaaatggagacagagtcatggatcgttg | |
| | acagacagtgggcccaggatctgacattgccatggcaatccggatccggaggcgtt | |
| | tggcgcgaaatgcatcatctagtcgagttcgaaccgccacatgccgctacaatcag | |
| | agtgttggccctaggcaatcaggagggaTCCTTGAAAACAGCTCTTACTGGCGCAA | |
| | TGAGGGTTACAAAGGACACAAATGACAACAACCTTTACAAACTACATGGTGGACAT | |
| | GTTTCTTGCAGAGTGAAATTGTCAGCTTTGACACTCAAGGGGACATCCTACAAAAT | |
| | ATGCACTGACAAAATGTTTTTTGTCAAGAACCCAACTGACACTGGCCATGGCACTG | |
| | TTGTGATGCAGGTGAAAGTGTCAAAAGGAGCCCCCTGCAGGATTCCAGTGATAGTA | |
| | GCTGATGATCTTACAGCGGCAATCAATAAAGGCATTTTGGTTACAGTTAACCCCAT | |
| | CGCCTCAACCAATGATGATGAAGTGCTGATTGAGGTGAACCCACCTTTTGGAGACA | |
| | GCTACATTATCGTTGGGAGAGGAGATTCACGTCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGCGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCGGATCAAGGATGCGCCATCAACTTTGGCAAGAGA | |
| | GAGCTCAAGTGCGGAGATGGTATCTTCATATTTAGAGACTCTGATGACTGGCTGAA | |
| | CAAGTACTCATACTATCCAGAAGATCCTGTGAAGCTTGCATCAATAGTGAAAGCCT | |
| | CT | |
| YF-Env- | ATGACTGGAAGAATGGGTGAAAGGCAACTCCAAAAGATTGAGAGATGGTTCGTGAG | 13 |
| WD | GAACCCCTTTTTTGCAGTGACGGCTCTGACCATTGCCTACCTTGTGGGAAGCAACA | |
| E gene | TGACGCAACGAGTCGTGATTGCCCTACTGGTCTTGGCTGTTGGTCCGGCCTACTCA | |
| within this | GCTCACTGCATTGGAATTACTGACAGGGATTTCATTGAGGGGGTGCATGGAGGAAC | |
| sequence | TTGGGTTTCAGCTACCCTGGAGCAAGACAAGTGTGTCACTGTTATGGCCCCTGACA | |
| | AGCCTTCATTGGACATCTCACTAGAGACAGTAGCCATTGATAGACCTGCTGAGGTG | |
| | AGGAAAGTGTGTTACAATGCAGTTCTCACTCATGTGAAGATTAATGACAAGTGCCC | |
| | CAGCACTGGAGAGGCCCACCTAGCTGAAGAGAACGAAGGGGACAATGCGTGCAAGC | |
| | GCACTTATTCTGATAGAGGCTGGGGCAATGGCTGTGGCCTATTTGGGAAAGGGAGC | |
| | ATTGTGGCATGCGCCAAATTCACTTGTGCCAAATCCATGAGTTTGTTTGAGGTTGA | |
| | TCAGACCAAAATTCAGTATGTCATCAGAGCACAATTGCATGTAGGGGCCAAGCAGG | |
| | AAAATTGGACTACCGACATTAAGACTCTCAAGTTTGATGCCCTGTCAGGCTCCCAG | |
| | GAAGTCGAGTTCATTGGGTATGGAAAAGCTACACTGGAATGCCAGGTGCAAACTGC | |
| | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCcttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagactgacataccaatggcataaagag | |
| | ggatcgtcaatcggtaagttgtttacacagactatgaaaggggtggagagattggc | |
| | cgttatgggcgataccgcttgggactttagttccgccggagggttttttactagcg | |
| | tcggaaaggggatacataccgtattcggatccgcttttcaggggttgttcggcgga | |
| | ctgaattggattacgaaagtgattatgggcgccgtacttatttgggtggggattaa | |
| | cactaggaatatgactatgtctatgtctatgatactagtcggagtgattatgatgt | |
| | ttctgtcattgggcgtaggcgctGATCAAGGATGCGCCATCAACTTTGGCAAGAGA | |
| | GAGCTCAAGTGCGGAGATGGTATCTTCATATTTAGAGACTCTGATGACTGGCTGAA | |
| | CAAGTACTCATACTATCCAGAAGATCCTGTGAAGCTTGCATCAATAGTGAAAGCCT | |
| | CT | |
| YF-Env- | ATGACTGGAAGAATGGGTGAAAGGCAACTCCAAAAGATTGAGAGATGGTTCGTGAG | 14 |
| DD | GAACCCCTTTTTTGCAGTGACGGCTCTGACCATTGCCTACCTTGTGGGAAGCAACA | |
| E gene | TGACGCAACGAGTCGTGATTGCCCTACTGGTCTTGGCTGTTGGTCCGGCCTACTCA | |
| within this | GCTCACTGCATTGGAATTACTGACAGGGATtttatcgagggggtgcatggcggaac | |
| sequence | ttgggttagcgctacactcgaacaggacaaatgcgttaccgttatggcccccgata | |
| | agcctagcctagacattagtctcgaaaccgttgcgatcgatagacccgccgaagtg | |
| | agaaaagtgtgttataacgccgtactgactcacgttaagattaacgacaaatgccc | |
| | tagtacaggcgaagcgcatctagccgaagagaacgagggcgataacgcatgcaaac | |
| | gtacttatagcgatagggggtgggggaacggatgcggattgttcggtaaggggtca | |
| | atcgtcgcatgcgctaagtttacatgcgctaagtctatgtcattgttcgaagtcga | |
| | tcagactaagattcagtacgtgattagagcgcaattgcatgtgggagcgaaacagg | |
| | agaattggactactgacattaagacactgaaattcgacgcccttagcggatcacag | |
| | gaggtcgagtttattgggtacggaaaagcgacactcgagtgtcaggtgcagactgc | |
| | cgttgactttggcaattcatacatagccgaaatggagacagagtcatggatcgttg | |
| | acagacagtgggcccaggatctgacattgccatggcaatccggatccggaggcgtt | |
| | tggcgcgaaatgcatcatctagtcgagttcgaaccgccacatgccgctacaatcag | |
| | agtgttggccctaggcaatcaggagggatcccttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagactgacataccaatggcataaagag | |
| | ggatcgtcaatcggtaagttgtttacacagactatgaaaggggtggagagattggc | |
| | cgttatgggcgataccgcttgggactttagttccgccggagggttttttactagcg | |
| | tcggaaaggggatacataccgtattcggatccgcttttcaggggttgttcggcgga | |
| | ctgaattggattacgaaagtgattatgggcgccgtacttatttgggtggggattaa | |
| | cactaggaatatgactatgtctatgtctatgatactagtcggagtgattatgatgt | |
| | ttctgtcattgggcgtaggcgctGATCAAGGATGCGCCATCAACTTTGGCAAGAGA | |
| | GAGCTCAAGTGCGGAGATGGTATCTTCATATTTAGAGACTCTGATGACTGGCTGAA | |
| | CAAGTACTCATACTATCCAGAAGATCCTGTGAAGCTTGCATCAATAGTGAAAGCCT | |
| | CT | |
| YF-Env- | ATGACTGGAAGAATGGGTGAAAGGCAACTCCAAAAGATTGAGAGATGGTTCGTGAG | 15 |
| DDDW | GAACCCCTTTTTTGCAGTGACGGCTCTGACCATTGCCTACCTTGTGGGAAGCAACA | |
| E gene | TGACGCAACGAGTCGTGATTGCCCTACTGGTCTTGGCTGTTGGTCCGGCCTACTCA | |
| within this | GCTCACTGCATTGGAATTACTGACAGGGATtttatcgagggggtgcatggcggaac | |
| sequence | ttgggttagcgctacactcgaacaggacaaatgcgttaccgttatggcccccgata | |
| | agcctagcctagacattagtctcgaaaccgttgcgatcgatagacccgccgaagtg | |
| | agaaaagtgtgttataacgccgtactgactcacgttaagattaacgacaaatgccc | |
| | tagtacaggcgaagcgcatctagccgaagagaacgagggcgataacgcatgcaaac | |
| | gtacttatagcgatagggggtgggggaacggatgcggattgttcggtaaggggtca | |
| | atcgtcgcatgcgctaagtttacatgcgctaagtctatgtcattgttcgaagtcga | |
| | tcagactaagattcagtacgtgattagagcgcaattgcatgtgggagcgaaacagg | |
| | agaattggactactgacattaagacactgaaattcgacgcccttagcggatcacag | |
| | gaggtcgagtttattgggtacggaaaagcgacactcgagtgtcaggtgcagactgc | |
| | cgttgactttggcaattcatacatagccgaaatggagacagagtcatggatcgttg | |
| | acagacagtgggcccaggatctgacattgccatggcaatccggatccggaggcgtt | |
| | tggcgcgaaatgcatcatctagtcgagttcgaaccgccacatgccgctacaatcag | |
| | agtgttggccctaggcaatcaggagggatcccttaaaaccgcattgactggcgcta | |
| | tgcgcgttactaaggacactaacgacaataacctatacaaactgcatggggggcat | |
| | gtgtcttgtagagtgaaattgtccgcccttacacttaaggggactagctataagat | |
| | atgcactgacaaaatgtttttcgttaaaaaccctaccgataccggacacggaacag | |
| | tcgttatgcaggtgaaagtgtcaaaaggcgcaccatgtaggatacccgtaatcgtt | |
| | gccgacgatctgactgccgcaatcaataaggggatactcgtgacagtgaaccctat | |
| | cgctagcactaacgacgacgaagtgttgatcgaagtgaatccaccttttggcgact | |
| | catacattatcgtaggcagaggcgatagtagaCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGGGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCGGATCAAGGATGCGCCATCAACTTTGGCAAGAGA | |
| | GAGCTCAAGTGCGGAGATGGTATCTTCATATTTAGAGACTCTGATGACTGGCTGAA | |
| | CAAGTACTCATACTATCCAGAAGATCCTGTGAAGCTTGCATCAATAGTGAAAGCCT | |
| | CT | |
| YF-Env- | ATGACTGGAAGAATGGGTGAAAGGCAACTCCAAAAGATTGAGAGATGGTTCGTGAG | 16 |
| Wt | GAACCCCTTTTTTGCAGTGACGGCTCTGACCATTGCCTACCTTGTGGGAAGCAACA | |
| E gene | TGACGCAACGAGTCGTGATTGCCCTACTGGTCTTGGCTGTTGGTCCGGCCTACTCA | |
| within this | GCTCACTGCATTGGAATTACTGACAGGGATTTCATTGAGGGGGTGCATGGAGGAAC | |
| sequence | TTGGGTTTCAGCTACCCTGGAGCAAGACAAGTGTGTCACTGTTATGGCCCCTGACA | |
| | AGCCTTCATTGGACATCTCACTAGAGACAGTAGCCATTGATAGACCTGCTGAGGTG | |
| | AGGAAAGTGTGTTACAATGCAGTTCTCACTCATGTGAAGATTAATGACAAGTGCCC | |
| | CAGCACTGGAGAGGCCCACCTAGCTGAAGAGAACGAAGGGGACAATGCGTGCAAGC | |
| | GCACTTATTCTGATAGAGGCTGGGGCAATGGCTGTGGCCTATTTGGGAAAGGGAGC | |
| | ATTGTGGCATGCGCCAAATTCACTTGTGCCAAATCCATGAGTTTGTTTGAGGTTGA | |
| | TCAGACCAAAATTCAGTATGTCATCAGAGCACAATTGCATGTAGGGGCCAAGCAGG | |
| | AAAATTGGACTACCGACATTAAGACTCTCAAGTTTGATGCCCTGTCAGGCTCCCAG | |
| | GAAGTCGAGTTCATTGGGTATGGAAAAGCTACACTGGAATGCCAGGTGCAAACTGC | |
| | GGTGGACTTTGGTAACAGTTACATCGCTGAGATGGAAACAGAGAGCTGGATAGTGG | |
| | ACAGACAGTGGGCCCAGGACTTGACCCTGCCATGGCAGAGTGGAAGTGGCGGGGTG | |
| | TGGAGAGAGATGCATCATCTTGTCGAATTTGAACCTCCGCATGCCGCCACTATCAG | |
| | AGTACTGGCCCTGGGAAACCAGGAAGGCTCCTTGAAAACAGCTCTTACTGGCGCAA | |
| | TGAGGGTTACAAAGGACACAAATGACAACAACCTTTACAAACTACATGGTGGACAT | |
| | GTTTCTTGCAGAGTGAAATTGTCAGCTTTGACACTCAAGGGGACATCCTACAAAAT | |
| | ATGCACTGACAAAATGTTTTTTGTCAAGAACCCAACTGACACTGGCCATGGCACTG | |
| | TTGTGATGCAGGTGAAAGTGTCAAAAGGAGCCCCCTGCAGGATTCCAGTGATAGTA | |
| | GCTGATGATCTTACAGCGGCAATCAATAAAGGCATTTTGGTTACAGTTAACCCCAT | |
| | CGCCTCAACCAATGATGATGAAGTGCTGATTGAGGTGAACCCACCTTTTGGAGACA | |
| | GCTACATTATCGTTGGGAGAGGAGATTCACGTCTCACTTACCAGTGGCACAAAGAG | |
| | GGAAGCTCAATAGGAAAGTTGTTCACTCAGACCATGAAAGGCGTGGAACGCCTGGC | |
| | CGTCATGGGAGACACCGCCTGGGATTTCAGCTCCGCTGGAGGGTTCTTCACTTCGG | |
| | TTGGGAAAGGAATTCATACGGTGTTTGGCTCTGCCTTTCAGGGGCTATTTGGCGGC | |
| | TTGAACTGGATAACAAAGGTCATCATGGGGGCGGTACTTATATGGGTTGGCATCAA | |
| | CACAAGAAACATGACAATGTCCATGAGCATGATCTTGGTAGGAGTGATCATGATGT | |
| | TTTTGTCTCTAGGAGTTGGGGCGGATCAAGGATGCGCCATCAACTTTGGCAAGAGA | |
| | GAGCTCAAGTGCGGAGATGGTATCTTCATATTTAGAGACTCTGATGACTGGCTGAA | |
| | CAAGTACTCATACTATCCAGAAGATCCTGTGAAGCTTGCATCAATAGTGAAAGCCT | |
| | CT | |

Full length genome sequences: 1-118=5′-NTR; 119-481=C (363 nts, 121AAs); 749-973=M (225 nts, 75AAs); 974-2452=E (1479 nts, 493AAs); 2453-3679=NS1 (1227 nts, 409AAs); 3680-4180=NS2a (501 nts, 167AAs); 4181-4570=NS2b (390 nts, 130AAs); 4571-6439=NS3 (1869 nts, 623AAs); 6440-7300=NS4a (861 nts, 287AAs); 7301-7636=NS4b (336 nts, 112AAs); 7637-10354=NS5 (2718 nts, 906AAs); 10355-10862=3′-NTR (508 nts).

The deoptimized YFV, wherein the E protein coding sequence is deoptimized of this invention, is useful in prophylactic and therapeutic compositions for reducing tumor size and treating malignant tumors in various organs, such as: breast, colon, bronchial passage, epithelial lining of the gastrointestinal, upper respiratory and genito-urinary tracts, liver, prostate, the brain, or any other human tissue. In various embodiments, the deoptimized YFV wherein the E protein coding sequence is deoptimized of the present invention are useful for reducing the size of solid tumors and treating solid tumors. In particular embodiments, the tumors treated or reduced in size is glioma, glioblastoma, adenocarcinoma, melanoma, or neuroblastoma. In various embodiments, the tumor is a triple-negative breast cancer.

The pharmaceutical compositions of this invention may further comprise other therapeutics for the prophylaxis of malignant tumors. For example, the deoptimized YFV wherein the E protein coding sequence is deoptimized of this invention may be used in combination with surgery, radiation therapy and/or chemotherapy. Furthermore, one or more deoptimized YFV wherein the E protein coding sequence is deoptimized may be used in combination with two or more of the foregoing therapeutic procedures. Such combination therapies may advantageously utilize lower dosages of the administered therapeutic agents, thus avoiding possible toxicities or adverse effects associated with the various monotherapies.

The pharmaceutical compositions of this invention comprise a therapeutically effective amount of one or more deoptimized YFV according to this invention, and a pharmaceutically acceptable carrier. By “therapeutically effective amount” is meant an amount capable of causing lysis of the cancer cells to cause tumor necrosis. By “pharmaceutically acceptable carrier” is meant a carrier that does not cause an allergic reaction or other untoward effect in patients to whom it is administered.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the deoptimized viral chimeras.

The compositions of this invention may be in a variety of forms. These include, for example, liquid dosage forms, such as liquid solutions, dispersions or suspensions, injectable and infusible solutions. The preferred form depends on the intended mode of administration and prophylactic or therapeutic application. The preferred compositions are in the form of injectable or infusible solutions.

### Methods of Generating Deoptimized YFV Genome, Deoptimized Infectious YF RNA, Deoptimized YF Virus

In various embodiments, the deoptimized YFV of the present invention can be synthesized by well-known recombinant DNA techniques. Any standard manual on DNA technology provides detailed protocols to produce the deoptimized viral chimeras of the invention.

This invention further provides a method of synthesizing any of the viruses described herein, the method comprising (a) identifying the target virus to be synthesized, (b) completely sequencing the target virus or locating the sequence on a publicly or privately available database, (c) de novo synthesis of DNA containing the coding and noncoding region of the genome as a complete plasmid known as an “infectious clone” or as individual pieces of synthetic DNA that can be joined using overlapping PCR. In further embodiments, the entire genome is substituted with the synthesized DNA. In still further embodiments, a portion of the genome is substituted with the synthesized DNA. In yet other embodiments, said portion of the genome is the capsid coding region.

In various embodiments, deoptimized YFV of the present invention is made by first generating a deoptimized viral genome, comprising performing reverse transcription polymerase chain reaction (“RT-PCR”) on a viral RNA from Yellow Fever Virus (YFV) to generate cDNA; performing polymerase chain reaction (“PCR”) to generate and amplify two or more overlapping cDNA fragments from the cDNA, wherein the two or more overlapping cDNA fragments collectively encode the YFV; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences.

In other embodiments, deoptimized YFV of the present invention is made by first generating a deoptimized viral genome, comprising performing polymerase chain reaction (“PCR”) to generate and amplify two or more overlapping cDNA fragments from cDNA encoding viral RNA from a YFV, wherein the two or more overlapping cDNA fragments collectively encode the YFV, wherein one or more overlapping cDNA fragments comprises a deoptimized sequence; performing overlapping and amplifying PCR to construct the deoptimized viral genome, wherein the deoptimized viral genome comprises one or more deoptimized sequences.

In other embodiments, deoptimized YFV of the present invention is made by first generating a deoptimized viral genome, comprising performing polymerase chain reaction (“PCR”) to generate and amplify two or more overlapping cDNA fragments from cDNA encoding viral RNA from a YFV, wherein the two or more overlapping cDNA fragments collectively encode the YFV; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences.

In various embodiments, the method further comprises extracting the viral RNA from the RNA virus prior to performing RT-PCR.

In various embodiments, the deoptimized sequences comprises (1) a recoded sequence having reduced codon pair bias compared to a corresponding sequence on the cDNA, (2) an increased number of CpG or UpA di-nucleotides compared to a corresponding sequence on the cDNA; or (3) at least 5 codons substituted with synonymous codons less frequently used, as discussed herein.

In various embodiments, performing PCR to generate and amplify two or more overlapping cDNA fragments from the cDNA comprises using two or more primer pairs, each pair specific for each of the overlapping cDNA fragments. In various embodiments, performing PCR to generate and amplify two or more overlapping cDNA fragments from the cDNA comprises using two or more primer pairs selected from Table 2.

In various embodiments, the length of the primers is about 15-55 base pairs (bp) in length. In various embodiments, the length of the primers is about 20-40 bp in length. In various embodiments, the length of the primers is about 20-30 bp in length. In various embodiments, the length of the primers is about 11-15, 16-20, 21-25, 26-30, 31-35, 36-40, 41-45, 46-50, 51-55, 56-60, or 61-65 bp in length.

In various embodiments, performing PCR to generate and amplify two or more overlapping cDNA fragments from the cDNA comprises using 5 or more primer pairs, each pair specific for each of the overlapping cDNA fragments. In various embodiments, the two or more overlapping cDNA fragments from the cDNA is 5 or more overlapping cDNA fragments and the 5 or more overlapping cDNA fragments collectively encode the RNA virus. In various embodiments, performing PCR to generate and amplify 5 or more overlapping cDNA fragments from the cDNA comprises using 5 or more primer pairs selected from Table 2.

In various embodiments, performing PCR to generate and amplify two or more overlapping cDNA fragments from the cDNA comprises using 8 or more primer pairs, each pair specific for each of the overlapping cDNA fragments. In various embodiments, the two or more overlapping cDNA fragments from the cDNA is 8 or more overlapping cDNA fragments and the 8 or more overlapping cDNA fragments collectively encode the RNA virus. In various embodiments, performing PCR to generate and amplify 8 or more overlapping cDNA fragments from the cDNA comprises using 8 or more primer pairs selected from Table 2.

In various embodiments, performing PCR to generate and amplify two or more overlapping cDNA fragments from the cDNA comprises using 10 or more primer pairs, each pair specific for each of the overlapping cDNA fragments. In various embodiments, the two or more overlapping cDNA fragments from the cDNA is 10 or more overlapping cDNA fragments and the 10 or more overlapping cDNA fragments collectively encode the RNA virus.

In various embodiments, performing PCR to generate and amplify two or more overlapping cDNA fragments from the cDNA comprises using 15 or more primer pairs, each pair specific for each of the overlapping cDNA fragments. In various embodiments, the two or more overlapping cDNA fragments from the cDNA is 15 or more overlapping cDNA fragments and the 15 or more overlapping cDNA fragments collectively encode the RNA virus.

In various embodiments, performing PCR to generate and amplify two or more overlapping cDNA fragments from the cDNA comprises using two or more primer pairs selected from Table 2.

TABLE 2

| | | SEQ | |
|---|---|---|---|
| | | ID | Primer Usage/PCR |
| Primer # | Primer Sequence | NO: | Product Size |
| 2557- YFVF1 F | AGTAAATCCTGTGTGCTAATTGAGGTG | 17 | for YFV Fragment 1 |
| | | | (998 bp) |
| 2520-YFVF1-R | TGTCAGTAATTCCAATGCAGTGAG | 18 | for YFV Fragment 1 |
| | | | (998 bp) |
| 2519-YFVF1-F | AGCTTATCATCGATAAGCTTGCTAGC | 19 | for YFV Fragment 1 |
| | | | containing phi2.5 T7 |
| | | | promoter (1046 bp) |
| 2520-YFVF1-R | TGTCAGTAATTCCAATGCAGTGAG | 20 | for YFV Fragment 1 |
| | | | containing phi2.5 T7 |
| | | | promoter (1046 bp) |
| 2521-YFVF2-F | ATGACTGGAAGAATGGGTGAAAGG | 21 | for YFV Fragment 2 |
| | | | (1794 bp) |
| 2522-YFVF2-R | AGAGGCTTTCACTATTGATGCAAGC | 22 | for YFV Fragment 2 |
| | | | (1794 bp) |
| 2523-YFVF3-F | ATCAAGGATGCGCCATCAACTTTG | 23 | for YFV Fragment 3 |
| | | | (1550 bp) |
| 2524-YFVF3-R | AAGTCTCACCTCAGCCATAGTGAC | 24 | for YFV Fragment 3 |
| | | | (1550 bp) |
| 2525-YFVF4-F | AACGCCTTGTGCTGACCCTAG | 25 | for YFV Fragment 4 |
| | | | (1596 bp) |
| 2526-YFVF4-R | TTGGTTCCAACATCCTGTAAGTTAG | 26 | for YFV Fragment 4 |
| | | | (1596 bp) |
| 2527-YFVF5-F | ATCTTGGCCGAGTGCGCACG | 27 | for YFV Fragment 5 |
| | | | (1598 bp) |
| 2528-YFVF5-R | TCGGGGATCACAACCACCATC | 28 | for YFV Fragment 5 |
| | | | (1598 bp) |
| 2529-YFVF6-F | TGCTGTTTATACTGGCTGGACTAC | 29 | for YFV Fragment 6 |
| | | | (1601 bp) |
| 2530-YFVF6-R | TGGCATGTATGGAGCTAACACC | 30 | for YFV Fragment 6 |
| | | | (1601 bp) |
| 2531-YFVF7-F | ATCATCACCTTCAAGGACAAAACTG | 31 | for YFV Fragment 7 |
| | | | (1600 bp) |
| 2532-YFVF7-R | ATCCGTGCTCAGTGAGCCATG | 32 | for YFV Fragment 7 |
| | | | (1600 bp) |
| 2533-YFVF8-F | AGCCTACATGGATGTCATAAGTC | 33 | for YFV Fragment 8 |
| | | | (1460 bp) |
| 2534-YFVF8-R | AGTGGTTTTGTGTTTGTCATCCAAAG | 34 | for YFV Fragment 8 |
| | | | (1460 bp) |
| 2862-YFVF5 F | ACG TGA AAT TCC ACA CAC AGG C | 35 | For YFV Fragment 5 |
| | | | (1537 bp) |
| 2863-YFVF5_R | ACT TGG TTG TCT TGG ATG GAC C | 36 | For YFV Fragment 5 |
| | | | (1537 bp) |
| 2529-YFVF6_F | TGC TGT TTA TAC TGG CTG GAC TAC | 37 | For YFV Fragment 6 |
| | | | (1601 bp) |
| 2530-YFVF6 R | TGG CAT GTA TGG AGC TAA CAC C | 38 | For YFV Fragment 6 |
| | | | (1601 bp) |
| 2531-YFVF7 F | ATCATCACCTTCAAGGACAAAACT G | 39 | For YFV Fragment 7 |
| | | | (1606 bp) |
| 2864-YFVF7 R | TGT TAC ATC CGT GCT CAG TGA G | 40 | For YFV Fragment 7 |
| | | | (1606 bp) |
| 2533-YFVF8 F | AGC CTA CAT GGA TGT CAT AAG TC | 41 | For YFV Fragment 8 |
| | | | (1460 bp) |
| 2865-YFVF8_R | AGT GGT TTT GTG TTT GTC ATC CAA AGG | 42 | For YFV Fragment 8 |
| | TCT GC | | (1460 bp) |

In various embodiments, the length of the overlap is about 40-400 bp. In various embodiments, the length of the overlap is about 200 bp. In various embodiments, the length of the overlap is about 40-100 bp. In various embodiments, the length of the overlap is about 100-200 bp. In various embodiments, the length of the overlap is about 100-150 bp. In various embodiments, the length of the overlap is about 150-200 bp. In various embodiments, the length of the overlap is about 200-250 bp. In various embodiments, the length of the overlap is about 200-300 bp. In various embodiments, the length of the overlap is about 300-400 bp.

In various embodiments, each of the one or more overlapping cDNA fragments comprising the deoptimized sequence comprises a sequence having one or more mutations relative to a corresponding sequence on the cDNA. In certain embodiments, there are 5 or more mutations. In certain embodiments, there are 10 or more mutations. In certain embodiments, there are 20 or more mutations. In certain embodiments there are 50 or more mutations. In certain embodiments there are 100 or more mutations. The one or more mutations can be a deletion, addition, substitution or combinations thereof.

In various embodiments, each of the one or more overlapping cDNA fragments comprising the deoptimized sequence comprises a sequence encoding an amino acid sequence having up to 2% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence that results in having up to 1.75% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence having up to 1.5% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence having up to 1.25% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence having up to 1% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence having up to 0.75% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence having up to 0.5% amino acid substitutions, additions or deletions relative to the amino acid sequence encoded by the corresponding sequence on the cDNA. In various embodiments, each of the one or more overlapping cDNA fragments comprising the deoptimized sequence comprises a sequence encoding an amino acid sequence that having up to 0.25% amino acid substitutions, additions or deletions relative to the amino acid sequence encoded by the corresponding sequence on the cDNA.

In various embodiments, performing overlapping PCR to construct the deoptimized viral genome is done on the two or more overlapping cDNA fragments at the same time. Thus, if there are 5 more overlapping cDNA fragments, overlapping PCR to construct the deoptimized viral genome is done on those 5 fragments at the same time. As further examples, if there are 8 more overlapping cDNA fragments, overlapping PCR to construct the deoptimized viral genome is done on those 8 fragments at the same time; if there are 10 more overlapping cDNA fragments, overlapping PCR to construct the deoptimized viral genome is done on those 10 fragments at the same time.

In various embodiments, the methods do not use an intermediate DNA clone, such as a plasmid, BAC or YAC. In various embodiments, the methods do not use a cloning host. In various embodiments, the methods do not include an artificial intron in the sequences; for example, to disrupt an offending sequence locus.

Various embodiments of the invention provide for a method of generating a deoptimized infectious YFV RNA, comprising: performing in vitro transcription of a deoptimized viral genome to generate a deoptimized RNA transcript.

In various embodiments, the method comprises generating the deoptimized viral genome in accordance with embodiments of the present invention before performing the in vitro transcription.

Thus, in various embodiments, the method comprises performing reverse transcription polymerase chain reaction (“RT-PCR”) on a viral RNA from a Yellow Fever virus to generate cDNA; performing polymerase chain reaction (“PCR”) to generate and amplify two or more overlapping cDNA fragments from the cDNA, wherein the two or more overlapping cDNA fragments collectively encode the YF virus; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript.

In other embodiments, the method comprises performing polymerase chain reaction (“PCR”) to generate and amplify two or more overlapping cDNA fragments from cDNA encoding viral RNA from a YF virus, wherein the two or more overlapping cDNA fragments collectively encode the YF virus, wherein one or more overlapping cDNA fragments comprises a deoptimized sequence; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript.

In other embodiments, the method comprises performing polymerase chain reaction (“PCR”) to generate and amplify two or more overlapping cDNA fragments from cDNA encoding viral RNA from a YF virus, wherein the two or more overlapping cDNA fragments collectively encode the YF virus; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript.

In various embodiments, the method further comprising extracting the viral RNA from the YF virus prior to performing RT-PCR.

Specific embodiments of the deoptimized viral genome and methods of generating the deoptimized viral genome are as provided above and below and are included in these embodiments for generating deoptimized infectious YFV RNA.

Various embodiments of the invention provide for a method of generating a deoptimized YF virus, comprising transfecting host cells with a quantity of a deoptimized infectious RNA; culturing the host cells; and collecting infection medium comprising the deoptimized virus.

In various embodiments, the method comprises performing reverse transcription polymerase chain reaction (“RT-PCR”) on a viral RNA from a Yellow Fever virus to generate cDNA; performing polymerase chain reaction (“PCR”) to generate and amplify two or more overlapping cDNA fragments from the cDNA, wherein the two or more overlapping cDNA fragments collectively encode the YF virus; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences; performing in vitro transcription of a deoptimized viral genome to generate a deoptimized RNA transcript; culturing the host cells; and collecting infection medium comprising the deoptimized virus.

In various embodiments, the method further comprises generating the quantity of deoptimized infectious RNA in accordance with various embodiments of the present invention before transfecting host cells with the quantity of the deoptimized infectious RNA. Thus, the invention comprises performing in vitro transcription of a deoptimized viral genome to generate a deoptimized RNA transcript; and transfecting host cells with a quantity of a deoptimized infectious RNA; culturing the host cells; and collecting infection medium comprising the deoptimized virus.

In other embodiments, the method comprises performing polymerase chain reaction (“PCR”) to generate and amplify two or more overlapping cDNA fragments from cDNA encoding viral RNA from a YF virus, wherein the two or more overlapping cDNA fragments collectively encode the YF virus, wherein one or more overlapping cDNA fragments comprises a deoptimized sequence; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript; culturing the host cells; and collecting infection medium comprising the deoptimized virus.

In other embodiments, the method comprises performing polymerase chain reaction (“PCR”) to generate and amplify two or more overlapping cDNA fragments from cDNA encoding viral RNA from a YF virus, wherein the two or more overlapping cDNA fragments collectively encode the YF virus; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript; culturing the host cells; and collecting infection medium comprising the deoptimized virus.

In various embodiments, the method further comprising extracting the viral RNA from the RNA virus prior to performing RT-PCR.

Specific embodiments of the deoptimized viral genome, methods of generating the deoptimized viral genome, and the infectious YFV RNA and generating the infectious YFV RNA are as provided above and below and are included in these embodiments for generating deoptimized YFV.

Example of host cells include, but are not limited to Vero E6 cells, MDCK cells, HeLa cells, Chicken embryo fibroblasts, embryonated chicken eggs, MRC-5 cells, WISTAR cells, PERC.6 cells, Huh-7 cells, BHK cells, MA-10⁴ cells, Vero cells, WI-38 cells, and HEK 293 cells.

### Immune and or Vaccines Compositions

Various embodiments provide for an immune composition for inducing an immune response in a subject, comprising: a deoptimized Yellow Fever Virus of the present invention. The deoptimized Yellow Fever Virus is any one of the deoptimized Yellow Fever Virus discussed herein. In various embodiments, the deoptimized Yellow Fever Virus of the present invention is a live-attenuated virus. In some embodiments the immune composition further comprises an acceptable excipient or carrier as described herein. In some embodiments, the immune composition further comprises a stabilizer as described herein. In some embodiments, the immune composition further comprise an adjuvant as described herein. In some embodiments, the immune composition further comprises sucrose, glycine or both. In various embodiments, the immune composition further comprises about sucrose (5%) and about glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the immune composition is provided at dosages of about 10³-10⁷ PFU. In various embodiments, the immune composition is provided at dosages of about 10⁴-10⁶ PFU. In various embodiments, the immune composition is provided at a dosage of about 10³ PFU. In various embodiments, the immune composition is provided at a dosage of about 10⁴ PFU. In various embodiments, the immune composition is provided at a dosage of about 10⁵ PFU. In various embodiments, the immune composition is provided at a dosage of about 10⁶ PFU. In various embodiments, the immune composition is provided at a dosage of about 10⁷ PFU. In various embodiments, the immune composition is provided at a dosage of about 10⁸ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU. In various embodiments, the immune composition is provided at a dosage of about 5×10⁸ PFU.

In various embodiments, the immune composition is provided at a dosage of about 3×10⁴ PFU. In various embodiments, the immune composition is provided at a dosage of about 3×10⁵ PFU. In various embodiments, the immune composition is provided at a dosage of about 3×10⁶ PFU. In various embodiments, the immune composition is provided at a dosage of about 3×10⁷ PFU. In various embodiments, the immune composition is provided at a dosage of about 3×10⁸ PFU.

In various embodiments, the immune composition is provided at a dosage of about 6.25×10⁵ PFU. In various embodiments, the immune composition is provided at a dosage of about 6.25×10⁶ PFU. In various embodiments, the immune composition is provided at a dosage of about 6.25×10⁷ PFU. In various embodiments, the immune composition is provided at a dosage of about 6.25×10⁸ PFU. In various embodiments, the immune composition is provided at a dosage of about 6.25×10⁹ PFU.

Various embodiments provide for a vaccine composition for inducing an immune response in a subject, comprising: a deoptimized Yellow Fever Virus of the present invention. The deoptimized Yellow Fever Virus is any one of the deoptimized Yellow Fever Virus discussed herein. In various embodiments, the deoptimized Yellow Fever Virus of the present invention is a live-attenuated virus. In some embodiments the vaccine composition further comprises an acceptable carrier or excipient as described herein. In some embodiments, the immune composition further comprises a stabilizer as described herein. In some embodiments, the vaccine composition further comprise an adjuvant as described herein. In some embodiments, the vaccine composition further comprises sucrose, glycine or both. In various embodiments, the vaccine composition further comprises sucrose (5%) and glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the vaccine composition is provided at dosages of about 10³-10⁷ PFU. In various embodiments, the vaccine composition is provided at dosages of about 10⁴-10⁶ PFU. In various embodiments, the vaccine composition is provided at a dosage of about 10³ PFU. In various embodiments, the vaccine composition is provided at a dosage of about 10⁴ PFU. In various embodiments, the vaccine composition is provided at a dosage of about 10⁵ PFU. In various embodiments, the vaccine composition is provided at a dosage of about 10⁶ PFU. In various embodiments, the vaccine composition is provided at a dosage of about 10⁷ PFU. In various embodiments, the vaccine composition is provided at a dosage of about 10⁸ PFU. In various embodiments, the vaccine composition is provided at a dosage of about 10⁹ PFU.

In various embodiments, the vaccine composition is provided at a dosage of about 5×10³ PFU. In various embodiments, the vaccine composition is provided at a dosage of about 5×10⁴ PFU. In various embodiments, the vaccine composition is provided at a dosage of about 5×10⁵ PFU. In various embodiments, the vaccine composition is provided at a dosage of about 5×10⁶ PFU. In various embodiments, the vaccine composition is provided at a dosage of about 5×10⁷ PFU.

In various embodiments, the vaccine composition is provided at a dosage of about 3×10⁴ PFU. In various embodiments, the vaccine composition is provided at a dosage of about 3×10⁵ PFU. In various embodiments, the vaccine composition is provided at a dosage of about 3×10⁶ PFU. In various embodiments, the vaccine composition is provided at a dosage of about 3×10⁷ PFU.

In various embodiments, the vaccine composition is provided at a dosage of about 6.25×10⁵ PFU. In various embodiments, the vaccine composition is provided at a dosage of about 6.25×10⁶ PFU. In various embodiments, the vaccine composition is provided at a dosage of about 6.25×10⁷ PFU. In various embodiments, the vaccine composition is provided at a dosage of about 6.25×10⁸ PFU. In various embodiments, the vaccine composition is provided at a dosage of about 6.25×10⁹ PFU.

Various embodiments provide for a vaccine composition for inducing a protective immune response in a subject, comprising: a deoptimized Yellow Fever Virus of the present invention. The deoptimized Yellow Fever Virus is any one of the deoptimized Yellow Fever Virus discussed herein. In various embodiments, the deoptimized Yellow Fever Virus of the present invention is a live-attenuated virus. In some embodiments the vaccine composition further comprises an acceptable carrier or excipient as described herein. In some embodiments, the vaccine composition further comprise an adjuvant as described herein. In some embodiments, the vaccine composition further comprises sucrose, glycine or both. In various embodiments, the vaccine composition further comprises sucrose (5%) and glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the vaccine composition is provided at dosages of about 10³-10⁷ PFU. In various embodiments, the vaccine composition is provided at dosages of about 10⁴-10⁶ PFU. In various embodiments, the vaccine composition is provided at a dosage of about 10³ PFU. In various embodiments, the vaccine composition is provided at a dosage of about 10⁴ PFU. In various embodiments, the vaccine composition is provided at a dosage of about 10⁵ PFU. In various embodiments, the vaccine composition is provided at a dosage of about 10⁶ PFU. In various embodiments, the vaccine composition is provided at a dosage of about 10⁷ PFU. In various embodiments, the vaccine composition is provided at a dosage of about 10⁸ PFU. In various embodiments, the vaccine composition is provided at a dosage of about 10⁹ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU.

In various embodiments, the immune composition is provided at a dosage of about 3×10⁴ PFU. In various embodiments, the immune composition is provided at a dosage of about 3×10⁵ PFU. In various embodiments, the immune composition is provided at a dosage of about 3×10⁶ PFU. In various embodiments, the immune composition is provided at a dosage of about 3×10⁷ PFU.

In various embodiments, the immune composition is provided at a dosage of about 6.25×10⁵ PFU. In various embodiments, the immune composition is provided at a dosage of about 6.25×10⁶ PFU. In various embodiments, the immune composition is provided at a dosage of about 6.25×10⁷ PFU. In various embodiments, the immune composition is provided at a dosage of about 6.25×10⁸ PFU. In various embodiments, the immune composition is provided at a dosage of about 6.25×10⁹ PFU.

It should be understood that an attenuated virus of the invention, where used to elicit an immune response in a subject (or protective immune response) or to prevent a subject from or reduce the likelihood of becoming afflicted with a virus-associated disease, can be administered to the subject in the form of a composition additionally comprising a pharmaceutically acceptable carrier or excipient. Pharmaceutically acceptable carriers and excipients are known to those skilled in the art and include, but are not limited to, one or more of 0.01-0.1M and preferably 0.05M phosphate buffer, phosphate-buffered saline (PBS), DMEM, L-15, a 10-25% sucrose solution in PBS, a 10-25% sucrose solution in DMEM, or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate, gelatin, recombinant human serum albumin, human serum albumin, and/or magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers or excipients can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments, the vaccine composition or immune composition is formulated for delivery intravenously, or intrathecally, subcutaneously, intramuscularly, intradermally or intranasally. In various embodiments, the vaccine composition or immune composition is formulated for delivery intranasally. In various embodiments, the vaccine composition or immune composition is formulated for delivery via a nasal drop or nasal spray.

As discussed, any one of the deoptimized Yellow Fever Virus of the present invention can be used in the immune compositions or vaccine compositions discussed herein.

In various embodiments, the E protein of the deoptimized YFV is encoded by a polynucleotide having SEQ ID NO:3.

In various embodiments, the E protein of the deoptimized YFV is encoded by a variant of a polynucleotide having SEQ ID NO:3, wherein the variant is not the YFV 17D sequence or wild type sequence.

In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:3. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of a polynucleotide having SEQ ID NO:3.

In various embodiments, the deoptimized YFV is encoded by YFV 17D, wherein the YFV 17D E protein coding sequence is replaced with the E protein coding sequence variant of a polynucleotide having SEQ ID NO:3, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:3, wherein the variant is not the YFV 17D sequence. In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the E protein of the deoptimized YFV is encoded by a polynucleotide having SEQ ID NO:4.

In various embodiments, the E protein of the deoptimized YFV is encoded by a variant of a polynucleotide having SEQ ID NO:4, wherein the variant is not the YFV 17D sequence or wild type sequence.

In particular embodiments, variant of a polynucleotide having SEQ ID NO:4 has at least 95%, 96%, 97%, 98% or 99% sequence identity to SEQ ID NO:4 In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:4. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:4.

In various embodiments, the deoptimized YFV is encoded by YFV 17D, wherein the YFV 17D E protein coding sequence is replaced with the E protein coding sequence variant of a polynucleotide having SEQ ID NO:4, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:4, wherein the variant is not the YFV 17D sequence.

In particular embodiments, variant of a polynucleotide having SEQ ID NO:4 has at least 95%, 96%, 97%, 98% or 99% sequence identity to SEQ ID NO:4. In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:5.

In various embodiments, the E protein of the deoptimized YFV is encoded by a variant of a polynucleotide having SEQ ID NO:5, wherein the variant is not the YFV 17D sequence or wild type sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:5. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:5.

In various embodiments, the deoptimized YFV is encoded by YFV 17D, wherein the YFV 17D E protein coding sequence is replaced with the E protein coding sequence variant of a polynucleotide having SEQ ID NO:5, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:5, wherein the variant is not the YFV 17D sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:6.

In various embodiments, the E protein of the deoptimized YFV is encoded by a variant of a polynucleotide having SEQ ID NO:6, wherein the variant is not the YFV 17D sequence or wild type sequence.

In particular embodiments, variant of a polynucleotide having SEQ ID NO:6 has at least 95%, 96%, 97%, 98% or 99% sequence identity to SEQ ID NO:6 In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:6. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:6.

In various embodiments, the deoptimized YFV is encoded by YFV 17D, wherein the YFV 17D E protein coding sequence is replaced with the E protein coding sequence variant of a polynucleotide having SEQ ID NO:6, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:6, wherein the variant is not the YFV 17D sequence.

In particular embodiments, variant of a polynucleotide having SEQ ID NO:6 has at least 95%, 96%, 97%, 98% or 99% sequence identity to SEQ ID NO:6. In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:7 (YF-WWDW).

In various embodiments, the E protein of the deoptimized YFV is encoded by a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 95%, 96%, 97%, 98% or 99% sequence identity to SEQ ID NO: 7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:7 (YF-WWDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence SEQ ID NO:7 (YF-WWDW).

In various embodiments, the deoptimized YFV is encoded by YFV 17D, wherein the YFV 17D E protein coding sequence is replaced with an E protein coding sequence variant of a polynucleotide having SEQ ID NO:7 (YF-WWDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with an E protein coding sequence a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 950%, 96%, 97%, 98% or 99% sequence identity to SEQ ID NO: 7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:8.

In various embodiments, the E protein of the deoptimized YFV is encoded by a variant of a polynucleotide having SEQ ID NO:8, wherein the variant is not the YFV 17D sequence or wild type sequence.

In particular embodiments, variant of a polynucleotide having SEQ ID NO:8 has at least 95%, 96%, 97%, 98% or 99% sequence identity to SEQ ID NO:8. In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:8. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:8.

In various embodiments, the deoptimized YFV is encoded by YFV 17D, wherein the YFV 17D E protein coding sequence is replaced with the E protein coding sequence variant of a polynucleotide having SEQ ID NO:8, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:8, wherein the variant is not the YFV 17D sequence.

In particular embodiments, variant of a polynucleotide having SEQ ID NO:8 has at least 95%, 96%, 97%, 98% or 99% sequence identity to SEQ ID NO:8. In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:9.

In various embodiments, the E protein of the deoptimized YFV is encoded by a variant of a polynucleotide having SEQ ID NO:9, wherein the variant is not the YFV 17D sequence or wild type sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9.

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:9. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:9.

In various embodiments, the deoptimized YFV is encoded by YFV 17D, wherein the YFV 17D E protein coding sequence is replaced with the E protein coding sequence variant of a polynucleotide having SEQ ID NO:9, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:9, wherein the variant is not the YFV 17D sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:12 (YF-DW).

In various embodiments, the E protein of the deoptimized YFV is encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO: 12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:12 (YF-DW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:12 (YF-DW).

In various embodiments, the deoptimized YFV is encoded by YFV 17D, wherein the YFV 17D E protein coding sequence is replaced with the E protein encoded by a variant of a polynucleotide having SEQ ID NO: 12 (YF-DW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO:12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:13 (YF-WD).

In various embodiments, the E protein of the deoptimized YFV is encoded by a variant of a polynucleotide having SEQ ID NO:13 (YF-DW), wherein the variant is not the YFV 17D sequence.

In particular embodiments, variant of a polynucleotide having SEQ ID NO:13 (YF-DW) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO:13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant of a polynucleotide having SEQ ID NO: 13 (YF-WD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:13 (YF-WD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:13 (YF-WD).

In various embodiments, the deoptimized YFV is encoded by YFV 17D, wherein the YFV 17D E protein coding sequence is replaced with the E protein encoded by a variant of a polynucleotide having SEQ ID NO: 13 (YF-WD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:13 (YF-WD), wherein the variant is not the YFV 17D sequence.

In particular embodiments, variant of a polynucleotide having SEQ ID NO:13 (YF-WD) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO:13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant SEQ ID NO: 13 (YF-WD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO: 14 (YF-DD).

In various embodiments, the E protein of the deoptimized YFV is encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO: 14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:14 (YF-DD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:14 (YF-DD).

In various embodiments, the deoptimized YFV is encoded by YFV 17D, wherein the YFV 17D E protein coding sequence is replaced with the E protein encoded by a variant of a polynucleotide having SEQ ID NO:14 (YF-DD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO:14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:15 (YF-DDDW).

In various embodiments, the E protein of the deoptimized YFV is encoded by a variant of a polynucleotide having SEQ ID NO:15 (YF-DDDW), wherein the variant is not the YFV 17D sequence.

In particular embodiments, variant of a polynucleotide having SEQ ID NO: 15 (YF-DDDW) has at least 95%, 96%, 97%, 98% or 99% sequence identity to SEQ ID NO:15. In various embodiments, the variant of a polynucleotide having SEQ ID NO:15 has up to 20 mutations in SEQ ID NO:15. In various embodiments, the variant of a polynucleotide having SEQ ID NO:15 has up to 10 mutations in SEQ ID NO: 15. In various embodiments, the variant of a polynucleotide having SEQ ID NO: 15 has up to 5 mutations in SEQ ID NO:15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:15 (YF-DDDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:15 (YF-DDDW).

In various embodiments, the deoptimized YFV is encoded by YFV 17D, wherein the YFV 17D E protein coding sequence is replaced with the E protein encoded by a variant of a polynucleotide having SEQ ID NO:15 (YF-DDDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO: 15 (YF-DDDW), wherein the variant is not the YFV 17D sequence. In particular embodiments, variant of a polynucleotide having SEQ ID NO: 15 (YF-DDDW) has at least 95%, 96%, 97%, 98% or 99% sequence identity to SEQ ID NO: 15. In various embodiments, the variant of a polynucleotide having SEQ ID NO: 15 has up to 20 mutations in SEQ ID NO: 15. In various embodiments, the variant of a polynucleotide having SEQ ID NO:15 has up to 10 mutations in SEQ ID NO:15. In various embodiments, the variant of a polynucleotide having SEQ ID NO: 15 has up to 5 mutations in SEQ ID NO:15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

### Prophylactic and Therapeutic Cancer Treatments

Various embodiments of the present invention relate to the production of deoptimized Yellow Fever viruses, wherein the E protein coding sequence is deoptimized, and compositions comprising these deoptimized Yellow Fever viruses that can be used as oncolytic therapy to treat different tumor types and methods of treating tumors and cancer by administering the deoptimized YFV virus, such as YFV 17D, YFV 17D-204, YFV 17DD, or YFV 17D-213.

### Treatment of Existing Cancer

Various embodiments of the present invention provide for a method of inducing an oncolytic effect on a tumor or cancer cell. In various embodiments, this type of treatment can be made when a subject has been diagnosed with cancer. The method comprises administering deoptimized YFV, wherein the E protein coding sequence is deoptimized to a subject in need thereof. The deoptimized YFV can be provided and administered in a composition comprising a pharmaceutically acceptable carrier or excipient as provided herein.

In various embodiments, the deoptimized YFV is deoptimized YFV 17D, wherein the E protein coding sequence is deoptimized as described herein.

In various embodiments, the deoptimized YFV is YFV 17D-204, YFV 17DD, or YFV 17D-213, wherein the E protein coding sequence is deoptimized as described herein.

In various embodiments, inducing an oncolytic effect on a malignant tumor results in treating the malignant tumor.

In various embodiments, the method of treatment further comprises administering a PD-1 inhibitor. In other embodiments, the method of treatment further comprises administering a PD-L1 inhibitor. In still other embodiments, the method of treatment further comprises administering both an PD-1 inhibitor and a PD-L1 inhibitor.

In various embodiments, the PD-1 inhibitor is an anti-PD1 antibody. In various embodiments, the PD-L1 inhibitor is an anti-PD-L1 antibody. Examples of PD-1 inhibitors and PD-L1 inhibitors that are used are provided herein.

In various embodiments, the method of treatment further comprises administering a chemotherapeutic agent. Examples of chemotherapeutic agents that are used are provided herein.

In various embodiments, the method of treatment further comprises administering a cancer immunotherapy. Examples of caner immunotherapy that are used are provided herein.

In various embodiments, the method of treatment further comprises administration of an additional therapeutic agent. Examples of therapeutic agents that may be used in accordance with various embodiments of the present invention include: anti-cancer drugs (including chemotherapeutic agents and antiproliferative agents), therapeutic viral particles, antimicrobials (e.g., antibiotics, antifungals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors). Additional examples are provided herein.

In various embodiments, the treatment of the malignant tumor decreases the likelihood of recurrence of the malignant tumor. In various embodiments, treating the malignant tumor reduces the tumor size. In various embodiments, it can also decrease the likelihood of having a second cancer that is different from the malignant tumor. If the subject develops a second cancer that is different from the malignant tumor and the treatment of the malignant tumor results in slowing the growth of the second cancer. In some embodiments, after remission of the malignant tumor, the subject develops a second cancer that is different from the malignant tumor and the treatment of the malignant tumor results in slowing the growth of the second cancer.

### Prime-Boost Treatments

Various embodiments of the present invention provide for a method of eliciting an immune response and inducing an oncolytic effect on a tumor or cancer cell, using a prime-boost-type treatment regimen. In various embodiments, eliciting the immune response and inducing an oncolytic effect on the tumor or cancer cell results in treating a malignant tumor.

A prime dose of the deoptimized YFV wherein the E protein coding sequence is deoptimized of the present invention is administered to elicit an initial immune response. Thereafter, a boost dose of deoptimized YFV of the present invention is administered to induce oncolytic effects on the tumor and/or to elicit an immune response comprising oncolytic effect against the tumor.

In various embodiments, the method comprises administering a prime dose of a deoptimized YFV, wherein the E protein coding sequence is deoptimized, of the present invention to a subject in need thereof, and administering one or more boost dose of the deoptimized YFV of the present invention to the subject in need thereof.

In various embodiments, the deoptimized YFV, wherein the E protein coding sequence is deoptimized is deoptimized YFV 17D-204, deoptimized YFV 17DD, or deoptimized YFV 17D-213.

In various embodiments, the prime dose is administered subcutaneously, intramuscularly, intradermally, intranasally or intravenously.

In various embodiments, the one or more boost dose is administered intratumorally, intravenously, intrathecally or intraneoplastically (directly into the tumor). A preferred mode of administration is directly to the tumor site.

The timing between the prime and boost dosages can vary, for example, depending on the type of cancer, the stage of cancer, and the patient's health. In various embodiments, the first of the one or more boost dose is administered about 2 weeks after the prime dose. That is, the prime dose is administered and about two weeks thereafter, the boost dose is administered.

In various embodiments, the one or more boost dose is administered about 1 week after a prime dose. In various embodiments, the one or more boost dose is administered about 2 weeks after a prime dose. In various embodiments, the one or more boost dose is administered about 3 weeks after a prime dose. In various embodiments, the one or more boost dose is administered about 4 weeks after a prime dose. In various embodiments, 2, 3, 4, 5, 6, 7, 8, 9, 10, 11, 12, 13, 14, or 15 boost doses are administered. In various embodiments, 1-5, 5-10, 10-15, 15-20, 20-25, 25-30, 30-35, 35-40, 40-45 or 45-50 boost doses are administered. In various embodiments, the intervals between the boost doses can be 1, 2, 3, 4, 5, 6, 7, 8, 9, or 10 weeks. In additional embodiments, the intervals between the boost doses can be 1, 2, 3, 4, 5, 6, 7, 8, 9, 10, 11 or 12 months. As a non-limiting example, the prime dose can be administered, about two weeks thereafter a first boost dose can be administered, about one month after the first boost dose, a second boost dose can be administered, about 6 months after the second boost dose, a third boost dose can be administered. As another non-limiting example, the prime dose can be administered, about two weeks thereafter 10 boost doses are administered at one dose per week. As another non-limiting example, the prime dose can be administered, about two weeks thereafter a first boost dose can be administered, about six months after the first boost dose, a second boost dose can be administered, about 12 months after the second boost dose, a third boost dose can be administered. In further embodiments, additional boost dosages can be periodically administered; for example, every year, every other year, every 5 years, every 10 years, etc.

In various embodiments, the dosage amount can vary between the prime and boost dosages. As a non-limiting example, the prime dose can contain fewer copies of the virus compare to the boost dose.

In other embodiments, the route of administration can vary between the prime and the boost dose. In a non-limiting example, the prime dose can be administered subcutaneously, and the boost dose can be administered via injection into the tumor; for tumors that are in accessible, or are difficult to access, the boost dose can be administered intravenously.

In various embodiments, the treatment further comprises administering a PD-1 inhibitor. In other embodiments, the treatment further comprises administering a PD-L1 inhibitor. In still other embodiments, the treatment further comprises administering both an PD-1 inhibitor and a PD-L1 inhibitor. In particular embodiments, the PD-1 inhibitor, the PD-L1 inhibitor, or both are administered during the treatment (boost) phase, and not during the priming phase.

In various embodiments, the PD-1 inhibitor is an anti-PD1 antibody. In various embodiments, the PD-L1 inhibitor is an anti-PD-L1 antibody. Examples of PD-1 inhibitors and PD-L1 inhibitors are provided herein.

In various embodiments, the method of treatment further comprises administering a chemotherapeutic agent. Examples of chemotherapeutic agents that are used are provided herein.

In various embodiments, the method of treatment further comprises administering a cancer immunotherapy. Examples of caner immunotherapy that are used are provided herein.

In various embodiments, the method of treatment further comprises administration of an additional therapeutic agent. Examples of therapeutic agents that may be used in accordance with various embodiments of the present invention include: anti-cancer drugs (including chemotherapeutic agents and antiproliferative agents), therapeutic viral particles, antimicrobials (e.g., antibiotics, antifungals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors). Additional examples are provided herein.

### Prime-Boost Treatment Before Having Cancer

Various embodiments of the present invention provide for a method of eliciting an immune response in a subject who does not have cancer and inducing an oncolytic effect on a tumor or cancer cell if and when the tumor or cancer cell develops in the subject. The method uses a prime-boost-type treatment regimen. In various embodiments, eliciting the immune response and inducing an oncolytic effect on the tumor or cancer cell results in treating a malignant tumor if and when the subject develops cancer.

A prime dose of deoptimized YFV, wherein the E protein coding sequence is deoptimized of the present invention is administered to elicit an initial immune response when the subject does not have cancer or when the subject is not believed to have cancer. The latter may be due to undetectable or undetected cancer.

Thereafter, in some embodiments, a boost dose of deoptimized YFV of the present invention is administered periodically to continue to elicit the immune response. For example, a boost dose can be administered about every 1, 2, 3, 4, 5, 6, 7, 8, 9, or 10 years. In particular embodiments, the boost dose can be administered about every 5 years.

Alternatively, in other embodiments, a boost dose of deoptimized YFV of the present invention is administered after the subject is diagnosed with cancer. For example, once the subject is diagnosed with cancer, a treatment regimen involving the administration of a boost dose can be started shortly thereafter to induce oncolytic effects on the tumor and/or to elicit an immune response comprising an oncolytic effect against the tumor. In further embodiments, additional boost doses can be administered to continue to treat the cancer.

In various embodiments, the deoptimized YFV, wherein the E protein coding sequence is deoptimized is deoptimized YFV 17D-204, deoptimized YFV 17DD, or deoptimized YFV 17D-213 as described herein.

While not wishing to be bound by any particular theory, or set regimen, it is believed that the prime dose and boost dose(s) “teach” the subject's immune system to recognize virus-infected cells. Thus, when the subject develops cancer and the boost dose is administered, the subject's immune system recognizes the virus infected cells; this time, the virus infected cells are the cancer cells. During the immune response to the virus infected cancer cells, the immune system is also primed with cancer antigens, and thus enhances the anti-cancer immunity as the immune system will also target the cells expressing the cancer antigens.

As such, in various embodiments, the treatment of the malignant tumor decreases the likelihood of recurrence of the malignant tumor. It can also decrease the likelihood of having a second cancer that is different from the malignant tumor. If the subject develops a second cancer that is different from the malignant tumor and the treatment of the malignant tumor results in slowing the growth of the second cancer. In some embodiments, after remission of the malignant tumor, the subject develops a second cancer that is different from the malignant tumor and the treatment of the malignant tumor results in slowing the growth of the second cancer.

One can think of the prime and boost doses as an anti-cancer vaccine, preparing the immune system to target treated tumor cells when cancer develops.

In various embodiments, the prime dose is administered subcutaneously, intramuscularly, intradermally, intranasally or intravenously.

In various embodiments, the one or more boost dose, when it is administered to a subject who does not have cancer, or is not suspected to have cancer, it is administered subcutaneously, intramuscularly, intradermally, intranasally or intravenously.

In various embodiments, the one or more boost dose, when it is administered to a subject who had been diagnosed with cancer, it is administered intratumorally, intravenously, intrathecally or intraneoplastically (directly into the tumor). A preferred mode of administration is directly to the tumor site.

The timing between the prime and boost dosages can vary, for example, depending on the type of cancer, the stage of cancer, and the patient's health. In various embodiments, the first of the one or more boost dose is administered about every 1, 2, 3, 4, 5, 6, 7, 8, 9, or 10 years after the prime dose, if the subject does not have cancer or is not suspected to have cancer. In particular embodiments, the boost dose is administered about every 5 years.

In various embodiments, for example, when the subject is diagnosed with cancer the one or more boost dose is administered after the diagnosis of cancer. In various embodiments, 2, 3, 4, or 5 boost doses are administered. In various embodiments, 2, 3, 4, 5, 6, 7, 8, 9, or 10 boost doses are administered. In various embodiments, the intervals between the boost doses can be 1, 2, 3, 4, 5, 6, 7, 8, 9, or 10 weeks. In additional embodiments, the intervals between the boost doses can be 1, 2, 3, 4, 5, 6, 7, 8, 9, 10, 11 or 12 months. As a non-limiting example, the prime dose can be administered, about five years thereafter, a first boost dose can be administered, about one year after the first boost dose, the subject is diagnosed with cancer, and a second boost dose can be administered, about 2 weeks after the second boost dose, a third boost dose can be administered, about 2 weeks after the third boost dose, a fourth boost dose can be administered, and about 1 month after the fourth boost dose a fifth boost dose can be administered. Once the cancer is determined to be in remission, additional periodic boost doses can be administered; for example, every 6 months, every year, every 2, years, every 3, years, every 4 years or every 5 years.

In various embodiments, the dosage amount can vary between the prime and boost dosages. As a non-limiting example, the prime dose can contain fewer copies of the virus compare to the boost dose.

In other embodiments, the route of administration can vary between the prime and the boost dose. In a non-limiting example, the prime dose can be administered subcutaneously, and the boost dose can be administered via injection into the tumor (when the subject has cancer); for tumors that are in accessible, or are difficult to access, the boost dose can be administered intravenously.

In various embodiments, subjects that receive these treatments (e.g., prime dose before having cancer, or prime and boost doses before having cancer, and then followed by boost doses after having cancer) can be a subject who are at a higher risk of developing cancer. Examples of such subject include but are not limited to, subjects with genetic dispositions (e.g., BRCA1 or BRCA2 mutation, TP53 mutations, PTEN mutations, KRAS mutations, c-Myc mutations, any mutation deemed by the National Cancer Institute as a cancer-predisposing mutation, etc.), family history of cancer, advanced age (e.g., 40, 45, 55, 65 years or older), higher than normal radiation exposure, prolonged sun exposure, history of tobacco use (e.g., smoking, chewing), history of alcohol abuse, history of drug abuse, a body mass index >25, history of a chronic inflammatory disease(s) (e.g., inflammatory bowel diseases, ulcerative colitis, Crohn disease, asthma, rheumatoid arthritis, etc.), history of immune suppression, history of chronic infections known to have a correlation to increased cancer risk (e.g., Hepatitis C, Hepatitis B, EBV, CMV, HPV, HIV, HTLV-1, MCPyV, H. pylori, etc.).

In various embodiments, subjects that receive these treatments (e.g., prime dose and boost dose before having cancer, or prime and boost doses before having cancer, and then followed by boost doses after having cancer) can be subjects who do not fall into the higher risk category but are prescribed the prime and boost doses by their clinician as a preventive measure for future cancer risk.

In various embodiments, the treatment further comprises administering a PD-1 inhibitor. In other embodiments, the treatment further comprises administering a PD-L1 inhibitor. In still other embodiments, the treatment further comprises administering both an PD-1 inhibitor and a PD-L1 inhibitor. In particular embodiments, the PD-1 inhibitor, the PD-L1 inhibitor, or both are administered during the treatment (boost) phase, and not during the priming phase.

In various embodiments, the PD-1 inhibitor is an anti-PD1 antibody. In various embodiments, the PD-L1 inhibitor is an anti-PD-L1 antibody. Examples of PD-1 inhibitors and PD-L1 inhibitors are provided herein.

In various embodiments, the method of treatment further comprises administering a chemotherapeutic agent. Examples of chemotherapeutic agents that are used are provided herein.

In various embodiments, the method of treatment further comprises administering a cancer immunotherapy. Examples of caner immunotherapy that are used are provided herein.

In various embodiments, the method of treatment further comprises administration of an additional therapeutic agent. Examples of therapeutic agents that may be used in accordance with various embodiments of the present invention include: anti-cancer drugs (including chemotherapeutic agents and antiproliferative agents), therapeutic viral particles, antimicrobials (e.g., antibiotics, antifungals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors). Additional examples are provided herein.

As discussed, any one of the deoptimized Yellow Fever Virus of the present invention as discussed herein can be used for the prophylactic and therapeutic cancer treatments. In various embodiments, the deoptimized YFV are provided in immune compositions or vaccine compositions.

In various embodiments, the E protein of the deoptimized YFV is encoded by a polynucleotide having SEQ ID NO:3.

In various embodiments, the E protein of the deoptimized YFV is encoded by a variant of a polynucleotide having SEQ ID NO:3, wherein the variant is not the YFV 17D sequence or wild type sequence.

In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:3. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of a polynucleotide having SEQ ID NO:3.

In various embodiments, the deoptimized YFV is encoded by YFV 17D, wherein the YFV 17D E protein coding sequence is replaced with the E protein coding sequence variant of a polynucleotide having SEQ ID NO:3, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:3, wherein the variant is not the YFV 17D sequence. In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the E protein of the deoptimized YFV is encoded by a polynucleotide having SEQ ID NO:4.

In various embodiments, the E protein of the deoptimized YFV is encoded by a variant of a polynucleotide having SEQ ID NO:4, wherein the variant is not the YFV 17D sequence or wild type sequence.

In particular embodiments, variant of a polynucleotide having SEQ ID NO:4 has at least 95%, 96%, 97%, 98% or 99% sequence identity to SEQ ID NO:4 In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:4. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:4.

In various embodiments, the deoptimized YFV is encoded by YFV 17D, wherein the YFV 17D E protein coding sequence is replaced with the E protein coding sequence variant of a polynucleotide having SEQ ID NO:4, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:4, wherein the variant is not the YFV 17D sequence.

In particular embodiments, variant of a polynucleotide having SEQ ID NO:4 has at least 95%, 96%, 97%, 98% or 99% sequence identity to SEQ ID NO:4. In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:5.

In various embodiments, the E protein of the deoptimized YFV is encoded by a variant of a polynucleotide having SEQ ID NO:5, wherein the variant is not the YFV 17D sequence or wild type sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:5. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:5.

In various embodiments, the deoptimized YFV is encoded by YFV 17D, wherein the YFV 17D E protein coding sequence is replaced with the E protein coding sequence variant of a polynucleotide having SEQ ID NO:5, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:5, wherein the variant is not the YFV 17D sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:6.

In various embodiments, the E protein of the deoptimized YFV is encoded by a variant of a polynucleotide having SEQ ID NO:6, wherein the variant is not the YFV 17D sequence or wild type sequence.

In particular embodiments, variant of a polynucleotide having SEQ ID NO:6 has at least 95%, 96%, 97%, 98% or 99% sequence identity to SEQ ID NO:6 In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:6. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:6.

In various embodiments, the deoptimized YFV is encoded by YFV 17D, wherein the YFV 17D E protein coding sequence is replaced with the E protein coding sequence variant of a polynucleotide having SEQ ID NO:6, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:6, wherein the variant is not the YFV 17D sequence.

In particular embodiments, variant of a polynucleotide having SEQ ID NO:6 has at least 95%, 96%, 97%, 98% or 99% sequence identity to SEQ ID NO:6. In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:7 (YF-WWDW).

In various embodiments, the E protein of the deoptimized YFV is encoded by a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 950%, 96%, 97%, 98% or 99% sequence identity to SEQ ID NO:7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:7 (YF-WWDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence SEQ ID NO:7 (YF-WWDW).

In various embodiments, the deoptimized YFV is encoded by YFV 17D, wherein the YFV 17D E protein coding sequence is replaced with an E protein coding sequence variant of a polynucleotide having SEQ ID NO:7 (YF-WWDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with an E protein coding sequence a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 950%, 96%, 97%, 98% or 99% sequence identity to SEQ ID NO: 7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:8.

In various embodiments, the E protein of the deoptimized YFV is encoded by a variant of a polynucleotide having SEQ ID NO:8, wherein the variant is not the YFV 17D sequence or wild type sequence.

In particular embodiments, variant of a polynucleotide having SEQ ID NO:8 has at least 95%, 96%, 97%, 98% or 99% sequence identity to SEQ ID NO:8 In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:8. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:8.

In various embodiments, the deoptimized YFV is encoded by YFV 17D, wherein the YFV 17D E protein coding sequence is replaced with the E protein coding sequence variant of a polynucleotide having SEQ ID NO:8, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:8, wherein the variant is not the YFV 17D sequence.

In particular embodiments, variant of a polynucleotide having SEQ ID NO:8 has at least 95%, 96%, 97%, 98% or 99% sequence identity to SEQ ID NO:8. In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:9.

In various embodiments, the E protein of the deoptimized YFV is encoded by a variant of a polynucleotide having SEQ ID NO:9, wherein the variant is not the YFV 17D sequence or wild type sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9.

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:9. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:9.

In various embodiments, the deoptimized YFV is encoded by YFV 17D, wherein the YFV 17D E protein coding sequence is replaced with the E protein coding sequence variant of a polynucleotide having SEQ ID NO:9, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:9, wherein the variant is not the YFV 17D sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:12 (YF-DW).

In various embodiments, the E protein of the deoptimized YFV is encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO:12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:12 (YF-DW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:12 (YF-DW).

In various embodiments, the deoptimized YFV is encoded by YFV 17D, wherein the YFV 17D E protein coding sequence is replaced with the E protein encoded by a variant of a polynucleotide having SEQ ID NO: 12 (YF-DW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO:12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:13 (YF-WD).

In various embodiments, the E protein of the deoptimized YFV is encoded by a variant of a polynucleotide having SEQ ID NO:13 (YF-DW), wherein the variant is not the YFV 17D sequence.

In particular embodiments, variant of a polynucleotide having SEQ ID NO:13 (YF-DW) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO:13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant of a polynucleotide having SEQ ID NO: 13 (YF-WD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:13 (YF-WD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:13 (YF-WD).

In various embodiments, the deoptimized YFV is encoded by YFV 17D, wherein the YFV 17D E protein coding sequence is replaced with the E protein encoded by a variant of a polynucleotide having SEQ ID NO: 13 (YF-WD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:13 (YF-WD), wherein the variant is not the YFV 17D sequence.

In particular embodiments, variant of a polynucleotide having SEQ ID NO:13 (YF-WD) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO: 13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant SEQ ID NO: 13 (YF-WD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:14 (YF-DD).

In various embodiments, the E protein of the deoptimized YFV is encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO: 14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:14 (YF-DD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:14 (YF-DD).

In various embodiments, the deoptimized YFV is encoded by YFV 17D, wherein the YFV 17D E protein coding sequence is replaced with the E protein encoded by a variant of a polynucleotide having SEQ ID NO:14 (YF-DD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO:14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:15 (YF-DDDW).

In various embodiments, the E protein of the deoptimized YFV is encoded by a variant of a polynucleotide having SEQ ID NO:15 (YF-DDDW), wherein the variant is not the YFV 17D sequence.

In particular embodiments, variant of a polynucleotide having SEQ ID NO: 15 (YF-DDDW) has at least 95%, 96%, 97%, 98% or 99% sequence identity to SEQ ID NO:15. In various embodiments, the variant of a polynucleotide having SEQ ID NO:15 has up to 20 mutations in SEQ ID NO:15. In various embodiments, the variant of a polynucleotide having SEQ ID NO:15 has up to 10 mutations in SEQ ID NO: 15. In various embodiments, the variant of a polynucleotide having SEQ ID NO: 15 has up to 5 mutations in SEQ ID NO: 15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:15 (YF-DDDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:15 (YF-DDDW).

In various embodiments, the deoptimized YFV is encoded by YFV 17D, wherein the YFV 17D E protein coding sequence is replaced with the E protein encoded by a variant of a polynucleotide having SEQ ID NO:15 (YF-DDDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO: 15 (YF-DDDW), wherein the variant is not the YFV 17D sequence. In particular embodiments, variant of a polynucleotide having SEQ ID NO: 15 (YF-DDDW) has at least 95%, 96%, 97%, 98% or 99% sequence identity to SEQ ID NO: 15. In various embodiments, the variant of a polynucleotide having SEQ ID NO: 15 has up to 20 mutations in SEQ ID NO: 15. In various embodiments, the variant of a polynucleotide having SEQ ID NO:15 has up to 10 mutations in SEQ ID NO:15. In various embodiments, the variant of a polynucleotide having SEQ ID NO: 15 has up to 5 mutations in SEQ ID NO:15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

### Inflammatory Response

In various embodiments, the administration of the deoptimized YFV, wherein the E protein coding sequence is deoptimized of the present invention to stimulate endogenous Type-1 interferon production in the subject which provides, in part, the therapeutic efficacy.

In various embodiments, the administration of the deoptimized YFV, wherein the E protein coding sequence is deoptimized of the present invention to maintain a therapeutically effective amount of Type-1 interferon production in the subject which provides, in part, the therapeutic efficacy.

In still other embodiments, the administration of the deoptimized YFV, wherein the E protein coding sequence is deoptimized of the present invention to activate of Type I Interferon in a subject to maintain ionizing radiation and chemotherapy sensitization in the subject.

In various embodiments the administration of the deoptimized YFV, wherein the E protein coding sequence is deoptimized of the present invention to recruit pro-inflammatory immune cells including CD45+ Leukocytes, Neutrophils, B-cells, CD4+ T-cells, and CD8+ immune cells to the site of cancer, which provides, in part, the therapeutic efficacy.

In various embodiments the administration of the deoptimized YFV, wherein the E protein coding sequence is deoptimized of the present invention to decrease anti-inflammatory immune cells such as FoxP3+T-regulatory cells or M2-Macrophages from the site of cancer, which provides, in part, the therapeutic efficacy.

In various embodiments, the treatment of the malignant tumor decreases the likelihood of recurrence of the malignant tumor. It can also decrease the likelihood of having a second cancer that is different from the malignant tumor. If the subject develops a second cancer that is different from the malignant tumor and the treatment of the malignant tumor results in slowing the growth of the second cancer. In some embodiments, after remission of the malignant tumor, the subject develops a second cancer that is different from the malignant tumor and the treatment of the malignant tumor results in slowing the growth of the second cancer.

### Methods of Eliciting an Immune Response or Vaccination

Various embodiments provide for a method of eliciting an immune response in a subject, comprising: administering to the subject a dose of an immune composition the present invention. The immune response elicited can be against YFV. The immune response elicited can also be a protective immune response against YFV. The immune composition is any one of the immune composition discussed herein. In various embodiments, the dose is a prophylactically effective or therapeutically effective dose.

In various embodiments, the immune composition is administered intravenously, or intrathecally, subcutaneously, intramuscularly, intradermally or intranasally. In various embodiments, the immune composition is administered intranasally. In various embodiments, the immune composition is administered via a nasal drop or nasal spray.

Various embodiments provide for a method of eliciting an immune response in a subject, comprising: administering to the subject a dose of a vaccine composition the present invention. The immune response elicited can be against YFV. The immune response elicited can also be a protective immune response against YFV. The vaccine composition is any one of the vaccine composition discussed herein. In various embodiments, the immune response is a protective immune response. In various embodiments, the dose is a prophylactically effective or therapeutically effective dose.

In various embodiments, the vaccine composition is administered intravenously, or intrathecally, subcutaneously, intramuscularly, intradermally or intranasally. In various embodiments, the vaccine composition is administered intranasally. In various embodiments, the vaccine composition is administered via a nasal drop or nasal spray.

Various embodiments provide for a method of eliciting an immune response in a subject, comprising: administering to the subject a dose of a deoptimized Yellow Fever Virus of the present invention. The immune response elicited can be against YFV. The immune response elicited can also be a protective immune response against YFV. The deoptimized Yellow Fever Virus is any one of the deoptimized Yellow Fever Virus discussed herein. In various embodiments, the immune response is a protective immune response. In various embodiments, the dose is a prophylactically effective or therapeutically effective dose.

In various embodiments, the dose is about 10³-10⁷ PFU. In various embodiments, the dose is about 10⁴-10⁶ PFU. In various embodiments, the dose is about 10³ PFU. In various embodiments, the dose is about 10⁴ PFU. In various embodiments, the dose is about 10⁵ PFU. In various embodiments, the dose is about 10⁶ PFU. In various embodiments, the dose is about 10⁷ PFU.

In various embodiments, the dose is about 5×10³ PFU. In various embodiments, the dose is about 5×10⁴ PFU. In various embodiments, the dose is about 5×10⁵ PFU. In various embodiments, the dose is about 5×10⁶ PFU. In various embodiments, the dose is about 5×10⁷ PFU.

In various embodiments, the dose is about 3×10⁴ PFU. In various embodiments, the dose is about 3×10⁵ PFU. In various embodiments, the dose is about 3×10⁶ PFU. In various embodiments, the dose is about 3×10⁷ PFU. In various embodiments, the dose is about 3×10⁶ PFU. In various embodiments, the dose is about 3×10⁸ PFU.

In various embodiments, the dose is about 6.25×10⁵ PFU. In various embodiments, the dose is about 6.25×10⁶ PFU. In various embodiments, the dose is about 6.25×10⁷ PFU. In various embodiments, the dose is about 6.25×10⁸ PFU. In various embodiments, the dose is about 6.25×10⁹ PFU.

In various embodiments, the deoptimized Yellow Fever Virus is administered intravenously, or intrathecally, subcutaneously, intramuscularly, intradermally or intranasally. In various embodiments, the deoptimized Yellow Fever Virus is administered intranasally. In various embodiments, the deoptimized Yellow Fever Virus is administered via a nasal drop or nasal spray.

Various embodiments provide for a method of eliciting an immune response in a subject, comprising: administering to the subject a prime dose of a deoptimized Yellow Fever Virus of the present invention; and administering to the subject one or more boost doses of a deoptimized Yellow Fever Virus of the present invention. The immune response elicited can be against YFV. The immune response elicited can also be a protective immune response against YFV. The deoptimized Yellow Fever Virus is any one of the deoptimized Yellow Fever Virus discussed herein. In various embodiments, the dose is a prophylactically effective or therapeutically effective dose.

In various embodiments, the prime dose and/or the one or more boost doses of the deoptimized Yellow Fever Virus is administered intravenously, or intrathecally, subcutaneously, intramuscularly, intradermally or intranasally. In various embodiments, the prime dose and/or the one or more boost doses of the deoptimized Yellow Fever Virus is administered intranasally. In various embodiments, the prime dose and/or the one or more boost doses of the deoptimized Yellow Fever Virus is administered via a nasal drop or nasal spray.

Various embodiments provide for a method of eliciting an immune response in a subject, comprising: administering to the subject a prime dose of an immune composition of the present invention; and administering to the subject one or more boost doses of an immune composition of the present invention. The immune response elicited can be against YFV. The immune response elicited can also be a protective immune response against YFV. The immune composition is any one of the immune composition discussed herein. In various embodiments, the dose is a prophylactically effective or therapeutically effective dose.

In various embodiments, the prime dose and/or the one or more boost doses of the immune composition is administered intravenously, or intrathecally, subcutaneously, intramuscularly, intradermally or intranasally. In various embodiments, the prime dose and/or the one or more boost doses of the immune composition is administered intranasally. In various embodiments, the prime dose and/or the one or more boost doses of the immune composition is administered via a nasal drop or nasal spray.

Various embodiments provide for a method of eliciting an immune response in a subject, comprising: administering to the subject a prime dose of a vaccine composition of the present invention; and administering to the subject one or more boost doses of a vaccine composition of the present invention. The immune response elicited can be against YFV. The immune response elicited can also be a protective immune response against YFV. The vaccine composition is any one of the vaccine composition discussed herein. In various embodiments, the dose is a prophylactically effective or therapeutically effective dose.

In various embodiments, the prime dose and/or the one or more boost doses of the vaccine composition is administered intravenously, or intrathecally, subcutaneously, intramuscularly, intradermally or intranasally. In various embodiments, the prime dose and/or the one or more boost doses of the vaccine composition is administered intranasally. In various embodiments, the prime dose and/or the one or more boost doses of the vaccine composition is administered via a nasal drop or nasal spray.

The timing between the prime and boost dosages can vary, for example, depending on the stage of infection or disease (e.g., non-infected, infected, number of days post infection), and the patient's health. In various embodiments, the one or more boost dose is administered about 2 weeks after the prime dose. That is, the prime dose is administered and about two weeks thereafter, a boost dose is administered. In various embodiments, the one or more boost dose is administered about 4 weeks after the prime dose. In various embodiments, the one or more boost dose is administered about 6 weeks after the prime dose. In various embodiments, the one or more boost dose is administered about 8 weeks after the prime dose. In various embodiments, the one or more boost dose is administered about 12 weeks after the prime dose. In various embodiments, the one or more boost dose is administered about 1-12 weeks after the prime dose.

In various embodiments, the one or more boost doses can be given as one boost dose. In other embodiments, the one or more boost doses can be given as a boost dose periodically. For example, it can be given quarterly, every 4 months, every 6 months, yearly, every 2 years, every 3 years, every 4 years, every 5 years, every 6 years, every 7 years, every 8 years, every 9 years, or every 10 years.

In various embodiments, the prime dose and boost does are each about 10³-10⁷ PFU. In various embodiments, the prime dose and boost does are each about 10⁴-10⁶ PFU. In various embodiments, the prime dose and boost does are each about 10³ PFU. In various embodiments, the prime dose and boost does are each about 10⁴ PFU. In various embodiments, the prime dose and boost does are each about 10⁵ PFU. In various embodiments, the prime dose and boost does are each about 10⁶ PFU. In various embodiments, the dose is about 10⁷ PFU. In various embodiments, the dose is about 10⁸ PFU. In various embodiments, the dose is about 10⁹ PFU.

In various embodiments, the prime dose and boost does are each about 5×10³ PFU. In various embodiments, the prime dose and boost does are each about 5×10⁴ PFU. In various embodiments, the prime dose and boost does are each about 5×10⁵ PFU. In various embodiments, the prime dose and boost does are each about 5×10⁶ PFU. In various embodiments, the prime dose and boost does are each about 5×10⁷ PFU.

In various embodiments, the prime dose and boost does are each about 3×10⁴ PFU. In various embodiments, the prime dose and boost does are each about 3×10⁵ PFU. In various embodiments, the prime dose and boost does are each about 3×10⁶ PFU. In various embodiments, the prime dose and boost does are each about 3×10⁷ PFU. In various embodiments, the prime dose and boost does are each about 3×10⁸ PFU.

In various embodiments, the prime dose and boost does are each about 6.25×10⁵ PFU. In various embodiments, the prime dose and boost does are each about 6.25×10⁶ PFU. In various embodiments, the prime dose and boost does are each about 6.25×10⁷ PFU. In various embodiments, the prime dose and boost does are each about 6.25×10⁸ PFU. In various embodiments, the prime dose and boost does are each about 6.25×10⁹ PFU.

In various embodiments, the dosage for the prime dose and the boost dose is the same.

In various embodiments, the dosage amount can vary between the prime and boost dosages. As a non-limiting example, the prime dose can contain fewer copies of the virus compared to the boost dose. For example, the prime dose is about 10³ PFU and the boost dose is about 10⁴-10⁶ PFU, or, the prime dose is about 10⁴ and the boost dose is about 10⁵-10⁷ PFU.

In various embodiments, wherein the boost dose is administered periodically, the subsequent boost doses can be less than the first boost dose.

As another non-limiting example, the prime dose can contain more copies of the virus compared to the boost dose.

In various embodiments, the immune response is a protective immune response. The protective response can reduce the chances of having Yellow Fever in a subject.

In various embodiments, the dose is a prophylactically effective or therapeutically effective dose.

As discussed, any one of the deoptimized Yellow Fever Virus of the present invention as discussed herein can be used for the method of eliciting an immune response to YFV methods of vaccinating against YFV. In various embodiments, the deoptimized YFV are provided in immune compositions or vaccine composition.

In various embodiments, the E protein of the deoptimized YFV is encoded by a polynucleotide having SEQ ID NO:3.

In various embodiments, the E protein of the deoptimized YFV is encoded by a variant of a polynucleotide having SEQ ID NO:3, wherein the variant is not the YFV 17D sequence or wild type sequence.

In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:3. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of a polynucleotide having SEQ ID NO:3.

In various embodiments, the deoptimized YFV is encoded by YFV 17D, wherein the YFV 17D E protein coding sequence is replaced with the E protein coding sequence variant of a polynucleotide having SEQ ID NO:3, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:3, wherein the variant is not the YFV 17D sequence. In particular embodiments, variant of a polynucleotide having SEQ ID NO:3 has at least 95%, 96%, 97%, 98% or 99% sequence identity to SEQ ID NO:3. In various embodiments, the variant of a polynucleotide having SEQ ID NO:3 has up to 20 mutations in SEQ ID NO:3. In various embodiments, the variant of a polynucleotide having SEQ ID NO:3 has up to 10 mutations in SEQ ID NO:3. In various embodiments, the variant of a polynucleotide having SEQ ID NO:3 has up to 5 mutations in SEQ ID NO:3.

In particular embodiments, the variant of a polynucleotide having SEQ ID NO:3 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:3 encodes a polypeptide sequence with 10-20 amino acid substitutions, deletions or additions as compared to the YFV 17D E protein amino acid sequence. In particular embodiments, variant of a polynucleotide having SEQ ID NO:3 encodes a polypeptide sequence with up to 1-9 amino acid substitutions, deletions or additions as compared to the YFV 17D E protein amino acid sequence. In particular embodiments, variant of a polynucleotide having SEQ ID NO:3 encodes a polypeptide sequence with up to 1-5 amino acid substitutions, deletions or additions as compared to the YFV 17D E protein amino acid sequence.

In various embodiments, the E protein of the deoptimized YFV is encoded by a polynucleotide having SEQ ID NO:4.

In various embodiments, the E protein of the deoptimized YFV is encoded by a variant of a polynucleotide having SEQ ID NO:4, wherein the variant is not the YFV 17D sequence or wild type sequence.

In particular embodiments, variant of a polynucleotide having SEQ ID NO:4 has at least 95%, 96%, 97%, 98% or 99% sequence identity to SEQ ID NO:4 In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:4. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:4.

In various embodiments, the deoptimized YFV is encoded by YFV 17D, wherein the YFV 17D E protein coding sequence is replaced with the E protein coding sequence variant of a polynucleotide having SEQ ID NO:4, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:4, wherein the variant is not the YFV 17D sequence.

In particular embodiments, variant of a polynucleotide having SEQ ID NO:4 has at least 95%, 96%, 97%, 98% or 99% sequence identity to SEQ ID NO:4 In various embodiments, the variant of a polynucleotide having SEQ ID NO:4 has up to 20 mutations in SEQ ID NO:4. In various embodiments, the variant of a polynucleotide having SEQ ID NO:4 has up to 10 mutations in SEQ ID NO:4. In various embodiments, the variant of a polynucleotide having SEQ ID NO:4 has up to 5 mutations in SEQ ID NO:4.

In particular embodiments, the variant of a polynucleotide having SEQ ID NO:4 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:4 encodes a polypeptide sequence with 10-20 amino acid substitutions, deletions or additions as compared to the YFV 17D E protein amino acid sequence. In particular embodiments, variant of a polynucleotide having SEQ ID NO:4 encodes a polypeptide sequence with up to 1-9 amino acid substitutions, deletions or additions as compared to the YFV 17D E protein amino acid sequence. In particular embodiments, variant of a polynucleotide having SEQ ID NO:4 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:5.

In various embodiments, the E protein of the deoptimized YFV is encoded by a variant of a polynucleotide having SEQ ID NO:5, wherein the variant is not the YFV 17D sequence or wild type sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:5. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:5.

In various embodiments, the deoptimized YFV is encoded by YFV 17D, wherein the YFV 17D E protein coding sequence is replaced with the E protein coding sequence variant of a polynucleotide having SEQ ID NO:5, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:5, wherein the variant is not the YFV 17D sequence.

In particular embodiments, variant of a polynucleotide having SEQ ID NO:5 has at least 95%, 96%, 97%, 98% or 99% sequence identity to SEQ ID NO:5. In various embodiments, the variant of a polynucleotide having SEQ ID NO:5 has up to 20 mutations in SEQ ID NO:5. In various embodiments, the variant of a polynucleotide having SEQ ID NO:5 has up to 10 mutations in SEQ ID NO:5. In various embodiments, the variant of a polynucleotide having SEQ ID NO:5 has up to 5 mutations in SEQ ID NO:5.

In particular embodiments, the variant of a polynucleotide having SEQ ID NO:5 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:5 encodes a polypeptide sequence with 10-20 amino acid substitutions, deletions or additions as compared to the YFV 17D E protein amino acid sequence. In particular embodiments, variant of a polynucleotide having SEQ ID NO:5 encodes a polypeptide sequence with up to 1-9 amino acid substitutions, deletions or additions as compared to the YFV 17D E protein amino acid sequence. In particular embodiments, variant of a polynucleotide having SEQ ID NO:5 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:6.

In various embodiments, the E protein of the deoptimized YFV is encoded by a variant of a polynucleotide having SEQ ID NO:6, wherein the variant is not the YFV 17D sequence or wild type sequence.

In particular embodiments, variant of a polynucleotide having SEQ ID NO:6 has at least 95%, 96%, 97%, 98% or 99% sequence identity to SEQ ID NO:6 In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:6. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:6.

In various embodiments, the deoptimized YFV is encoded by YFV 17D, wherein the YFV 17D E protein coding sequence is replaced with the E protein coding sequence variant of a polynucleotide having SEQ ID NO:6, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:6, wherein the variant is not the YFV 17D sequence.

In particular embodiments, variant of a polynucleotide having SEQ ID NO:6 has at least 95%, 96%, 97%, 98% or 99% sequence identity to SEQ ID NO:6. In various embodiments, the variant of a polynucleotide having SEQ ID NO:6 has up to 20 mutations in SEQ ID NO:6. In various embodiments, the variant of a polynucleotide having SEQ ID NO:6 has up to 10 mutations in SEQ ID NO:6. In various embodiments, the variant of a polynucleotide having SEQ ID NO:6 has up to 5 mutations in SEQ ID NO:6.

In particular embodiments, the variant of a polynucleotide having SEQ ID NO:6 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:6 encodes a polypeptide sequence with 10-20 amino acid substitutions, deletions or additions as compared to the YFV 17D E protein amino acid sequence. In particular embodiments, variant of a polynucleotide having SEQ ID NO:6 encodes a polypeptide sequence with up to 1-9 amino acid substitutions, deletions or additions as compared to the YFV 17D E protein amino acid sequence. In particular embodiments, variant of a polynucleotide having SEQ ID NO:6 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:7 (YF-WWDW).

In various embodiments, the E protein of the deoptimized YFV is encoded by a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 950%, 96%, 97%, 98% or 99% sequence identity to SEQ ID NO:7. In various embodiments, the variant of a polynucleotide having SEQ ID NO:7 has up to 20 mutations in SEQ ID NO: 7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:7 (YF-WWDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence SEQ ID NO:7 (YF-WWDW).

In various embodiments, the deoptimized YFV is encoded by YFV 17D, wherein the YFV 17D E protein coding sequence is replaced with an E protein coding sequence variant of a polynucleotide having SEQ ID NO:7 (YF-WWDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with an E protein coding sequence a variant of a polynucleotide having SEQ ID NO:7 (YF-WWDW), wherein the variant is not the YFV 17D sequence.

In particular embodiments, variant of a polynucleotide having SEQ ID NO:7 (YF-WWDW) has at least 95%, 96%, 97%, 98% or 99% sequence identity to SEQ ID NO: 7. In various embodiments, the variant of a polynucleotide having SEQ ID NO:7 has up to 20 mutations in SEQ ID NO:7. In various embodiments, the variant of a polynucleotide having SEQ ID NO:7 has up to 10 mutations in SEQ ID NO:7. In various embodiments, the variant of a polynucleotide having SEQ ID NO:7 has up to 5 mutations in SEQ ID NO:7.

In particular embodiments, the variant of a polynucleotide having SEQ ID NO:7 (YF-WWDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with 10-20 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-9 amino acid substitutions, deletions or additions as compared to the YFV 17D E protein amino acid sequence. In particular embodiments, variant of a polynucleotide having SEQ ID NO:7 (YF-WWDW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:8.

In various embodiments, the E protein of the deoptimized YFV is encoded by a variant of a polynucleotide having SEQ ID NO:8, wherein the variant is not the YFV 17D sequence or wild type sequence.

In particular embodiments, variant of a polynucleotide having SEQ ID NO:8 has at least 95%, 96%, 97%, 98% or 99% sequence identity to SEQ ID NO:8 In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:8. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:8.

In various embodiments, the deoptimized YFV is encoded by YFV 17D, wherein the YFV 17D E protein coding sequence is replaced with the E protein coding sequence variant of a polynucleotide having SEQ ID NO:8, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:8, wherein the variant is not the YFV 17D sequence.

In particular embodiments, variant of a polynucleotide having SEQ ID NO:8 has at least 95%, 96%, 97%, 98% or 99% sequence identity to SEQ ID NO:8. In various embodiments, the variant of a polynucleotide having SEQ ID NO:8 has up to 20 mutations in SEQ ID NO:8. In various embodiments, the variant of a polynucleotide having SEQ ID NO:8 has up to 10 mutations in SEQ ID NO:8. In various embodiments, the variant of a polynucleotide having SEQ ID NO:8 has up to 5 mutations in SEQ ID NO:8.

In particular embodiments, the variant of a polynucleotide having SEQ ID NO:8 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:8 encodes a polypeptide sequence with 10-20 amino acid substitutions, deletions or additions as compared to the YFV 17D E protein amino acid sequence. In particular embodiments, variant of a polynucleotide having SEQ ID NO:8 encodes a polypeptide sequence with up to 1-9 amino acid substitutions, deletions or additions as compared to the YFV 17D E protein amino acid sequence. In particular embodiments, variant of a polynucleotide having SEQ ID NO:8 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:9.

In various embodiments, the E protein of the deoptimized YFV is encoded by a variant of a polynucleotide having SEQ ID NO:9, wherein the variant is not the YFV 17D sequence or wild type sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9.

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein coding sequence of SEQ ID NO:9. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence of SEQ ID NO:9.

In various embodiments, the deoptimized YFV is encoded by YFV 17D, wherein the YFV 17D E protein coding sequence is replaced with the E protein coding sequence variant of a polynucleotide having SEQ ID NO:9, wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein coding sequence variant of a polynucleotide having SEQ ID NO:9, wherein the variant is not the YFV 17D sequence.

In particular embodiments, variant of a polynucleotide having SEQ ID NO:9 has at least 95%, 96%, 97%, 98% or 99% sequence identity to SEQ ID NO:9. In various embodiments, the variant of a polynucleotide having SEQ ID NO:9 has up to 20 mutations in SEQ ID NO:9. In various embodiments, the variant of a polynucleotide having SEQ ID NO:9 has up to 10 mutations in SEQ ID NO:9. In various embodiments, the variant of a polynucleotide having SEQ ID NO:9 has up to 5 mutations in SEQ ID NO:9

In particular embodiments, the variant of a polynucleotide having SEQ ID NO:9 encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:9 encodes a polypeptide sequence with 10-20 amino acid substitutions, deletions or additions as compared to the YFV 17D E protein amino acid sequence. In particular embodiments, variant of a polynucleotide having SEQ ID NO:9 encodes a polypeptide sequence with up to 1-9 amino acid substitutions, deletions or additions as compared to the YFV 17D E protein amino acid sequence. In particular embodiments, variant of a polynucleotide having SEQ ID NO:9 encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:12 (YF-DW).

In various embodiments, the E protein of the deoptimized YFV is encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO: 12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:12 (YF-DW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:12 (YF-DW).

In various embodiments, the deoptimized YFV is encoded by YFV 17D, wherein the YFV 17D E protein coding sequence is replaced with the E protein encoded by a variant of a polynucleotide having SEQ ID NO: 12 (YF-DW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:12 (YF-DW), wherein the variant is not the YFV 17D sequence.

In particular embodiments, variant of a polynucleotide having SEQ ID NO:12 (YF-DW) has at least 95%, 96%, 97%, 98% or 99% sequence identity to SEQ ID NO: 12. In various embodiments, the variant of a polynucleotide having SEQ ID NO: 12 has up to 20 mutations in SEQ ID NO:12. In various embodiments, the variant of a polynucleotide having SEQ ID NO:12 has up to 10 mutations in SEQ ID NO:12. In various embodiments, the variant of a polynucleotide having SEQ ID NO: 12 has up to 5 mutations in SEQ ID NO:12.

In particular embodiments, the variant of a polynucleotide having SEQ ID NO: 12 (YF-DW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:12 (YF-DW) encodes a polypeptide sequence with 10-20 amino acid substitutions, deletions or additions as compared to the YFV 17D E protein amino acid sequence. In particular embodiments, variant of a polynucleotide having SEQ ID NO:12 (YF-DW) encodes a polypeptide sequence with up to 1-9 amino acid substitutions, deletions or additions as compared to the YFV 17D E protein amino acid sequence. In particular embodiments, variant of a polynucleotide having SEQ ID NO: 12 (YF-DW) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:13 (YF-WD).

In various embodiments, the E protein of the deoptimized YFV is encoded by a variant of a polynucleotide having SEQ ID NO:13 (YF-DW), wherein the variant is not the YFV 17D sequence.

In particular embodiments, variant of a polynucleotide having SEQ ID NO:13 (YF-DW) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO:13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant of a polynucleotide having SEQ ID NO: 13 (YF-WD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:13 (YF-WD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:13 (YF-WD).

In various embodiments, the deoptimized YFV is encoded by YFV 17D, wherein the YFV 17D E protein coding sequence is replaced with the E protein encoded by a variant of a polynucleotide having SEQ ID NO: 13 (YF-WD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:13 (YF-WD), wherein the variant is not the YFV 17D sequence.

In particular embodiments, variant of a polynucleotide having SEQ ID NO:13 (YF-WD) has at least 95%, 96%, 97%, 98% or 99% sequence identity to SEQ ID NO: 13. In various embodiments, the variant of a polynucleotide having SEQ ID NO: 13 has up to 20 mutations in SEQ ID NO: 13. In various embodiments, the variant of a polynucleotide having SEQ ID NO:13 has up to 10 mutations in SEQ ID NO:13. In various embodiments, the variant of a polynucleotide having SEQ ID NO: 13 has up to 5 mutations in SEQ ID NO:13.

In particular embodiments, the variant of a polynucleotide having SEQ ID NO:13 (YF-WD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:13 (YF-WD) encodes a polypeptide sequence with 10-20 amino acid substitutions, deletions or additions as compared to the YFV 17D E protein amino acid sequence. In particular embodiments, variant of a polynucleotide having SEQ ID NO:13 (YF-WD) encodes a polypeptide sequence with up to 1-9 amino acid substitutions, deletions or additions as compared to the YFV 17D E protein amino acid sequence. In particular embodiments, variant SEQ ID NO: 13 (YF-WD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:14 (YF-DD).

In various embodiments, the E protein of the deoptimized YFV is encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO: 14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:14 (YF-DD). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:14 (YF-DD).

In various embodiments, the deoptimized YFV is encoded by YFV 17D, wherein the YFV 17D E protein coding sequence is replaced with the E protein encoded by a variant of a polynucleotide having SEQ ID NO:14 (YF-DD), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO:14 (YF-DD), wherein the variant is not the YFV 17D sequence.

In particular embodiments, variant of a polynucleotide having SEQ ID NO: 14 (YF-DD) has at least 95%, 96%, 97%, 98% or 99% sequence identity to SEQ ID NO: 14. In various embodiments, the variant of a polynucleotide having SEQ ID NO: 14 has up to 20 mutations in SEQ ID NO:14. In various embodiments, the variant of a polynucleotide having SEQ ID NO:14 has up to 10 mutations in SEQ ID NO:14. In various embodiments, the variant of a polynucleotide having SEQ ID NO: 14 has up to 5 mutations in SEQ ID NO:14.

In particular embodiments, the variant of a polynucleotide having SEQ ID NO:14 (YF-DD) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:14 (YF-DD) encodes a polypeptide sequence with 10-20 amino acid substitutions, deletions or additions as compared to the YFV 17D E protein amino acid sequence. In particular embodiments, variant of a polynucleotide having SEQ ID NO: 14 (YF-DD) encodes a polypeptide sequence with up to 1-9 amino acid substitutions, deletions or additions as compared to the YFV 17D E protein amino acid sequence. In particular embodiments, variant of a polynucleotide having SEQ ID NO:14 (YF-DD) encodes a polypeptide sequence with up to 1-5 amino acid substitutions, deletions or additions as compared to the YFV 17D E protein amino acid sequence.

In various embodiments, the E protein of the deoptimized YFV is encoded by SEQ ID NO:15 (YF-DDDW).

In various embodiments, the E protein of the deoptimized YFV is encoded by a variant of a polynucleotide having SEQ ID NO:15 (YF-DDDW), wherein the variant is not the YFV 17D sequence.

In particular embodiments, variant of a polynucleotide having SEQ ID NO: 15 (YF-DDDW) has at least 95%, 96%, 97%, 98% or 99% sequence identity to SEQ ID NO:15. In various embodiments, the variant of a polynucleotide having SEQ ID NO:15 has up to 20 mutations in SEQ ID NO:15. In various embodiments, the variant of a polynucleotide having SEQ ID NO:15 has up to 10 mutations in SEQ ID NO: 15. In various embodiments, the variant of a polynucleotide having SEQ ID NO: 15 has up to 5 mutations in SEQ ID NO: 15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

In various embodiments, the deoptimized YFV is encoded by YFV 17D, wherein the YFV 17D E protein coding sequence is replaced with the E protein encoded by SEQ ID NO:15 (YF-DDDW). In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by SEQ ID NO:15 (YF-DDDW).

In various embodiments, the deoptimized YFV is encoded by YFV 17D, wherein the YFV 17D E protein coding sequence is replaced with the E protein encoded by a variant of a polynucleotide having SEQ ID NO:15 (YF-DDDW), wherein the variant is not the YFV 17D sequence. In various embodiments, the deoptimized YFV is encoded by YFV 17D-204, YFV 17DD, or YFV 17D-213, wherein the YFV 17D-204, YFV 17DD, or YFV 17D-213 E protein coding sequence is replaced with the E protein encoded by a variant of a polynucleotide having SEQ ID NO: 15 (YF-DDDW), wherein the variant is not the YFV 17D sequence. In particular embodiments, variant of a polynucleotide having SEQ ID NO: 15 (YF-DDDW) has at least 95%, 96%, 97%, 98% or 99% sequence identity to SEQ ID NO: 15. In various embodiments, the variant of a polynucleotide having SEQ ID NO: 15 has up to 20 mutations in SEQ ID NO: 15. In various embodiments, the variant of a polynucleotide having SEQ ID NO:15 has up to 10 mutations in SEQ ID NO:15. In various embodiments, the variant of a polynucleotide having SEQ ID NO: 15 has up to 5 mutations in SEQ ID NO:15.

In particular embodiments, the variant of a polynucleotide having SEQ ID NO:15 (YF-DDDW) encodes a polypeptide sequence with up to 20, 15, 10, or 5 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with 10-20 amino acid substitutions, deletions or additions as compared to the YFV 17D E protein amino acid sequence. In particular embodiments, variant of a polynucleotide having SEQ ID NO:15 (YF-DDDW) encodes a polypeptide sequence with up to 1-9 amino acid substitutions, deletions or additions as compared to the YFV 17D E protein amino acid sequence. In particular embodiments, variant of a polynucleotide having SEQ ID NO: 15 (YF-DDDW) encodes a polypeptide sequence with up to 1-5 amino acid substitutions, deletions or additions as compared to the YFV 17D E protein amino acid sequence.

### Kits

The present invention is also directed to a kit to vaccinate a subject, to elicit an immune response or to elicit a protective immune response in a subject. The kit is useful for practicing the inventive method of elicit an immune response or to elicit a protective immune response. The kit is an assemblage of materials or components, including at least one of the inventive compositions. Thus, in some embodiments the kit contains a composition including any one of the deoptimized Yellow Fever Virus discussed herein, any one of the immune compositions discussed herein, or any one of the vaccine compositions discussed herein of the present invention. Thus, in some embodiments the kit contains unitized single dosages of the composition including the deoptimized YFV, wherein the E protein coding sequence is deoptimized, the immune compositions, or the vaccine compositions of the present invention as described herein; for example, each vial contains enough for a dose of about 10³-10⁷ PFU of the deoptimized Yellow Fever Virus, or more particularly, 10⁴-10⁶ PFU of the deoptimized Yellow Fever Virus, 10⁴ PFU of the deoptimized Yellow Fever Virus, 10⁵ PFU of the deoptimized Yellow Fever Virus, 10⁶ PFU of the deoptimized Yellow Fever Virus, 107 PFU of the deoptimized Yellow Fever Virus, 10⁸ PFU of the deoptimized Yellow Fever Virus, or 10⁹ PFU of the deoptimized Yellow Fever Virus; or more particularly, 5×10⁴-5×10⁶ PFU of deoptimized Yellow Fever Virus, 5×10⁴ PFU of the deoptimized Yellow Fever Virus, 5×10⁵ PFU of deoptimized Yellow Fever Virus, or 5×10⁶ PFU of the deoptimized Yellow Fever Virus,

3×10⁴ PFU of the deoptimized Yellow Fever Virus, 3×10⁵ PFU of deoptimized Yellow Fever Virus, 3×10⁶ PFU of the deoptimized Yellow Fever Virus, 3×10⁷ PFU of the deoptimized Yellow Fever Virus, or 3×10⁸ PFU of the deoptimized Yellow Fever Virus, 6.25×10⁴ PFU of the deoptimized Yellow Fever Virus, 6.25×10⁵ PFU of deoptimized Yellow Fever Virus, 6.25×10⁶ PFU of the deoptimized Yellow Fever Virus, 6.25×10⁷ PFU of the deoptimized Yellow Fever Virus, 6.25×10⁸ PFU of the deoptimized Yellow Fever Virus, or 6.25×10⁹ PFU of the deoptimized Yellow Fever Virus. In various embodiments, the kit contains multiple dosages of the composition including the deoptimized Yellow Fever Virus, the immune compositions, or the vaccine compositions of the present invention as described herein; for example, if the kit contains 10 dosages per vial, each vial contains about 10×10³-10⁷ PFU of the deoptimized Yellow Fever Virus, or more particularly, 10×10⁴-10⁶ PFU of the deoptimized Yellow Fever Virus, 10×10⁴ PFU of the deoptimized Yellow Fever Virus, 10×10⁵ PFU of the deoptimized Yellow Fever Virus, or 10×10⁶ PFU of the deoptimized Yellow Fever Virus, or more particularly, 50×10⁴-50×10⁶ PFU of the deoptimized Yellow Fever Virus, 50×10⁴ PFU of the deoptimized Yellow Fever Virus, 50×10⁵ PFU of the deoptimized Yellow Fever Virus, or 50×10⁶ PFU of the deoptimized Yellow Fever Virus.

The exact nature of the components configured in the inventive kit depends on its intended purpose. For example, some embodiments are configured for the purpose of vaccinating a subject, for eliciting an immune response or for eliciting a protective immune response in a subject. In one embodiment, the kit is configured particularly for the purpose of prophylactically treating mammalian subjects. In another embodiment, the kit is configured particularly for the purpose of prophylactically treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to vaccinate a subject, to elicit an immune response or to elicit a protective immune response in a subject. For example, for nasal administration, instructions for use can include but are not limited to instructions for the subject to blow the nose and tilt the head back, instructions for the subject reposition the head to avoid having composition dripping outside of the nose or down the throat, instructions for administering about 0.25 mL comprising the dosage into each nostril; instructions for the subject to sniff gently, and/or instructions for the subject to not blow the nose for a period of time; for example, about 60 minutes. Further instructions can include instruction for the subject to not take any immunosuppressive medications.

Optionally, the kit also contains other useful components, such as, diluents, buffers, pharmaceutically acceptable carriers, syringes, droppers, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by known methods, preferably to provide a sterile, contaminant-free environment. The packaging materials employed in the kit are those customarily utilized in vaccines. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing deoptimized Yellow Fever Virus, the immune compositions, or the vaccine compositions of the present invention as described herein. The packaging material generally has an external label which indicates the contents and/or purpose of the kit and/or its components.

### PD-1 Inhibitors and PD-L1 Inhibitors

Examples of anti-PD1 antibodies that can be used as discussed herein include but are not limited to pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, and tislelizumab.

Additional examples of PD-1 inhibitors include but are not limited PF-06801591, anti-PD1 antibody expressing pluripotent killer T lymphocytes (PIK-PD-1), and autologous anti-EGFRvIII 4SCAR-IgT cells.

Examples of anti-PD-L1 antibody include but are not limited to BGB-A333, CK-301, FAZ053, KN035, MDX-1105, MSB2311, SHR-1316, atezolizumab, avelumab, durvalumab, BMS-936559, and CK-301. An additional example of an anti-PD-L1 inhibitor is M7824.

### Chemotherapeutic Agents

Examples of chemotherapeutic agents that can be used as discussed herein include but are not limited to taxanes (paclitaxel, nab-paclitaxel, docetaxel), platinum based therapies (cisplatin), gemcitabine, doxorubicin, or cyclophosphamide.

Additional examples of chemotherapeutic agent that can be sued as discussed herein include but are not limited cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic akylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

### Cancer Immunotherapies

Examples of chemotherapeutic agents that can be used as discussed herein include but are not limited to CTLA-4 blockade, LAG-3 blockade, and agonist of the CD226/TIGIT axis.

### Additional Therapeutic Agents

Examples of additional therapeutic agents that can be used as discussed herein include but are not limited to anti-cancer drugs (including chemotherapeutic agents and antiproliferative agents), therapeutic viral particles, antimicrobials (e.g., antibiotics, antifungals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors).

Examples of antiproliferative agents include but are not limited to alkylating agents, antimetabolites, enzymes, biological response modifiers, hormones and antagonists, androgen inhibitors (e.g., flutamide and leuprolide acetate), antiestrogens (e.g., tamoxifen citrate and analogs thereof, toremifene, droloxifene and roloxifene), Additional examples of antiproliferative agents include, but are not limited to levamisole, gallium nitrate, granisetron, sargramostim strontium-89 chloride, filgrastim, pilocarpine, dexrazoxane, and ondansetron.

### Routes of Administration

In additional to those discussed above, therapeutic oncolytic deoptimized YFV 17D virus (or deoptimized YFV 17D-204, deoptimized YFV 17DD, or deoptimized YFV 17D-213 as described herein) can be delivered intratumorally, intravenously, intrathecally or intraneoplastically (directly into the tumor). A preferred mode of administration is directly to the tumor site. The inoculum of virus applied for therapeutic purposes can be administered in an exceedingly small volume ranging between 1-10 μl.

It will be apparent to those of skill in the art that the therapeutically effective amount of deoptimized YFV 17D virus (or deoptimized YFV 17D-204, deoptimized YFV 17DD, or deoptimized YFV 17D-213 as described herein) of this invention can depend upon the administration schedule, the unit dose of deoptimized YFV 17D virus (or deoptimized YFV 17D-204, deoptimized YFV 17DD, or deoptimized YFV 17D-213 as described herein) administered, whether the deoptimized YFV 17D virus (or deoptimized YFV 17D-204, deoptimized YFV 17DD, or deoptimized YFV 17D-213 as described herein) is administered in combination with other therapeutic agents, the status and health of the patient. In various embodiments, a therapeutically effective amount of 4.74 log 10+/−2 log 10 of deoptimized YFV 17D virus of this invention is administered.

The therapeutically effective amounts of oncolytic recombinant virus can be determined empirically and depend on the maximal amount of the recombinant virus that can be administered safely, and the minimal amount of the recombinant virus that produces efficient oncolysis.

Therapeutic inoculations of oncolytic deoptimized YFV 17D virus (or deoptimized YFV 17D-204, deoptimized YFV 17DD, or deoptimized YFV 17D-213 as described herein) can be given repeatedly, depending upon the effect of the initial treatment regimen. Should the host's immune response to the oncolytic deoptimized YFV 17D virus (or deoptimized YFV 17D-204, deoptimized YFV 17DD, or deoptimized YFV 17D-213 as described herein), administered initially limit its effectiveness, additional injections of an oncolytic deoptimized viruses with a different deoptimized viruses' serotype can be made. The host's immune response to deoptimized YFV 17D virus (or deoptimized YFV 17D-204, deoptimized YFV 17DD, or deoptimized YFV 17D-213 as described herein) can be easily determined serologically. It will be recognized, however, that lower or higher dosages than those indicated above according to the administration schedules selected.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Synthesis of Deoptimized Yellow Fever Virus

Codon pair deoptimized cassettes are introduced into the 17D viral genome by reverse genetics methods to “over-attenuate” the resulting virus. The over-attenuation provides a safety “buffer” that will allow to absorb potential de-attenuating effects of mutations that may occur upon virus adaptation when switching the manufacturing substrate of the vaccine from chick embryos to cell culture.

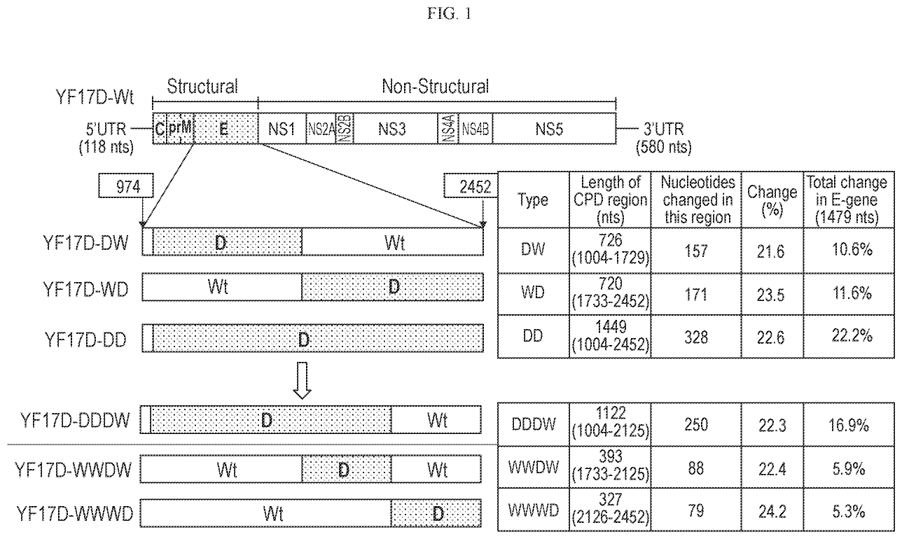

The published full length Yellow Fever Virus Vaccine (17D) genome sequence (Genbank Accession #JN628279, as of Jun. 28, 2021, herein incorporated by reference as though fully set forth) was divided in silico into 8 fragments with overlapping region at both ends. Fragments 1 and 3-8 correspond to the backbone 17D genome and are constant in the virus designs describe in this example. Fragment 2, encoding the E glycoprotein was deoptimized. See FIG. 1. Four versions of Fragment 2 (all encoding same amino acid sequence) were initially synthesized. F2-WW represents the sequence of the YF vaccine strain 17D. A synthetic 17D virus carrying the F2-WW cassette corresponds to a cloned version of the current 17D vaccine strain. In F2-DW, and F2-WD, either the first half or the second half of the E-glycoprotein are deoptimized, respectively. Introduction of F2-DW, and F2-WD into the 17D genome produces vaccine candidates YF-DW and YF-WD, respectively. F2-DD contains a wholly deoptimized E-glycoprotein, and the resulting YF-DD virus is expected to be the most highly attenuated vaccine candidate of the four viruses (YF-WW, YF-DW, YF-WD, YF-DD) currently contemplated. The recovery of YF-DD is described herein. However, the recovery method is applicable to YF-WW, YF-DW, YF-WD, YF-DDDW, YF-WWDW, YF-17D-WD-E-153N, YF-17D-WWDW-E-153N, and other YF deoptimized virus candidates.

The seven backbone fragments F1, F3-8, and four variations of F2 were synthesized de novo (BioBasic, Markham Ontario) and delivered as sequence confirmed plasmids (in low copy number vector pBR322).

Upon receiving synthetic plasmids from BioBasic, all fragments were PCR amplified and purified. Full length overlapping PCR were performed to obtain full length YF-DD DNA genome flanked by 3′ T7 RNA polymerase promoter. T7 in vitro transcription was used to generate infectious full length YF-DD genome RNA genome, which was used to recover YF-DD virus by transfection in animal origin free Vero (WHO 10-87) cells.

The above procedures were repeated with an additional version of F2. F2-DDDW contains a longer deoptimized region, wherein approximately the first ¾^{th} of the E-glycoprotein is deoptimized, as shown in FIG. 1.

### Experimental Procedures:

Cells—Vero WHO 10-87 (MCB+19 passages); animal origin free culture

Medium and reagents used: OptiPRO SFM, DMEM, NEB Q5, DPBS, mMESSAGE mMACHINE™ T7 Transcription Kit, Lipofectamine™ MessengerMAX™ Transfection Reagent

### PCR for Each Fragment

NEB Q5 polymerase was used to amplify all 8 genome fragments, synthesized by BioBasics, as building blocks for downstream overlapping PCR. 1 ng of each plasmids works as templates, amplified with gene specific primers (0.2 uM) in a 40 ul system. All PCR products were purified by DNAland Gel Extraction PCR Purification 2-in-1 Kit.

### Overlapping PCR for Full Length YF-DD

After purifying each PCR products, a mix of 0.02 pmol of each DNA fragment were used to generate full length YF-DD by overlapping PCR. Reaction volume was kept as 20 ul. Conditions were: 98° C. for 30 sec, and 72° C. for 4 min 30 sec for 10 cycles. No primers were used at this step.

After the initial step, 2 ul of overlapping PCR product were mixed with 0.1 uM Forward primer #2519 and Reverse primer #2534, as well as 2×Q5 to amplify the full length YF-DD. Reaction conditions were: 98° C. for 10 sec, 60° C. for 45 sec, and 72° C. for 5 min 30 sec, for 15 cycles. The final 11 kb full length YF-DD was gel checked. Full length products were further purified by DNAland Gel Extraction PCR Purification 2-in-1 Kit.

### Diagnostic PCR Check

16 diagnostic PCRs were used to confirm that the F2-DD PCR building block as well as the final full length YF-DD DNA genome carry the intended deoptimized F2 sequence, and rule out presence of 17D sequence in the F2 region (E domain).

### RNA Synthesis

HiScribe™ T7 In Vitro Transcription Kit (NEB) were used to generate full length YF-DD RNA. 2 ul of GTP, UTP, CTP (each at 100 mM concentration, 0.4 ul of ATP (100 mM), 4 ul 40 mM m7G(5′)ppp(5′) RNA Cap Structure Analog (NEB) NA synthesis were set at 37° C. for 3 hours. 2 ul of RNA were gel checked.

### Transfection

In vitro synthesized YF-DD RNA was used in transfection. Vero cells, seeded on 4×35 mm dishes. For transfection, 3 ul/7 ul RNA were mixed with 3.5 ul/7 ul Lipofectamine MessengerMAX mRNA Transfection Reagent for 5 min, and transferred to Vero cells grown in DMEM+OptiPRO. Mock transfected dishes received the same amount of Lipofectamine, without RNA. Medium were changed every 2-3 days until Day 12 post transfection. Cell death were monitored daily.

### Virus Passage

Supernatants from Day 4, Day 7 and Day 12 post transfection dishes were collected and used to infect fresh Vero Cells.

### YF Staining

To visualize YF-DD virus-infected cells, mouse monoclonal anti-Flavivirus Group Antigen Antibody, clone D1-4G2-4-15 (ATCC® HB-112), in conjunction with HRP-labeled goat anti-mouse secondary antibody and VECTOR VIP chromog 11 monolayers on Day 12 post transfection, or Day 8 post infection.

### Results & Discussion

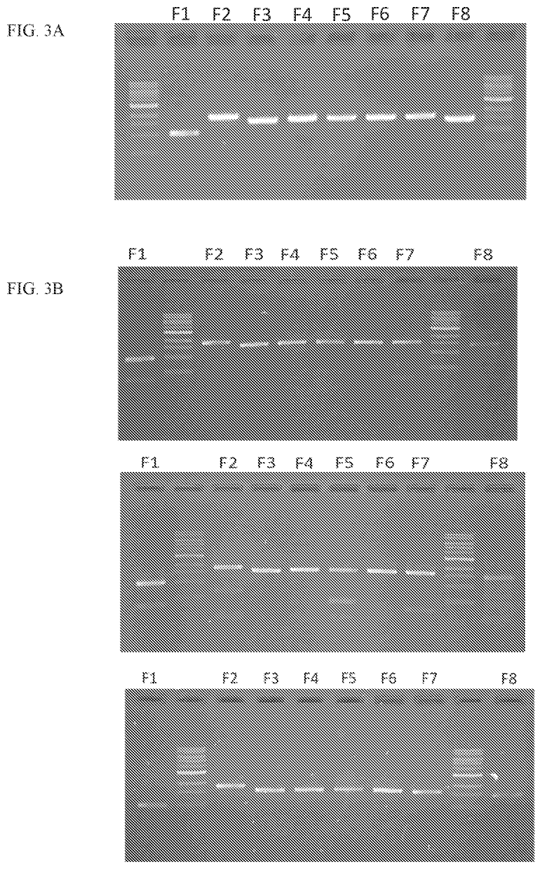

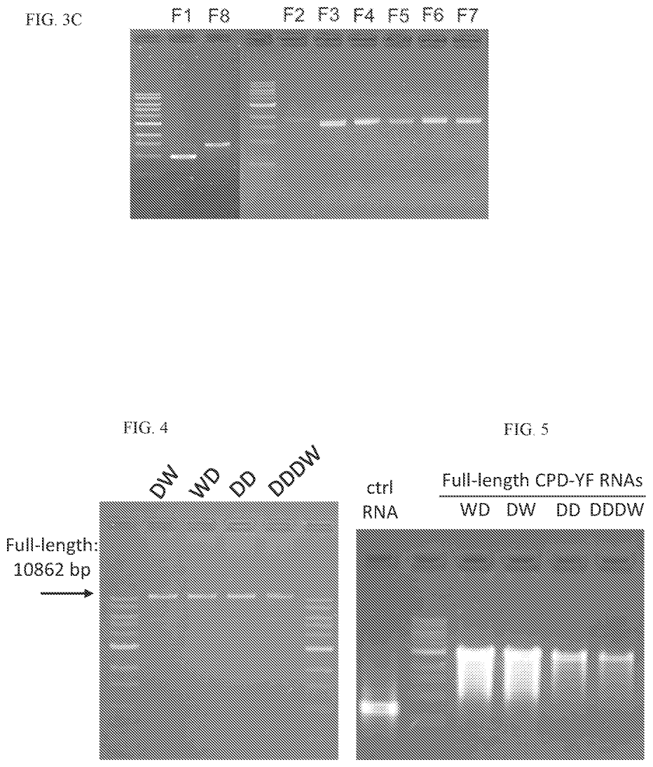

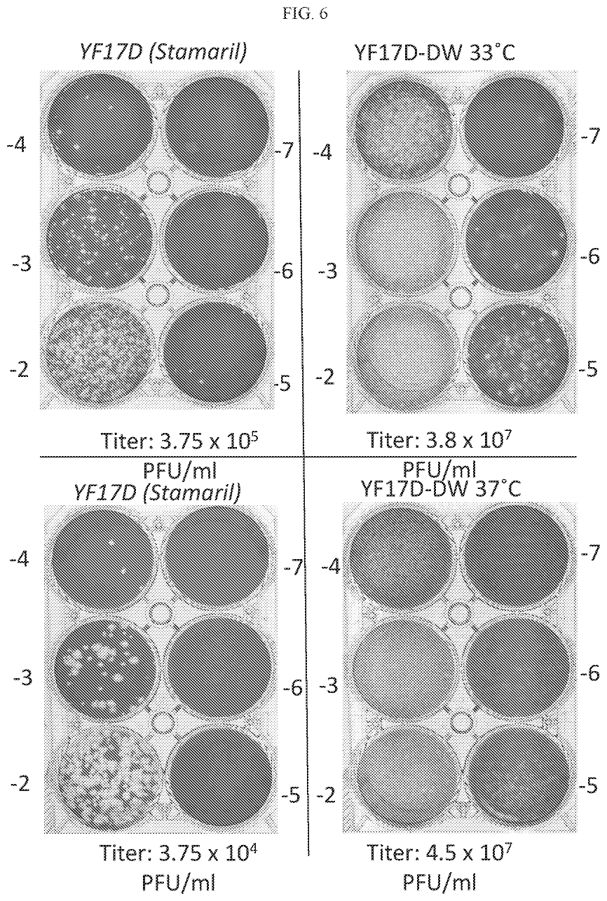

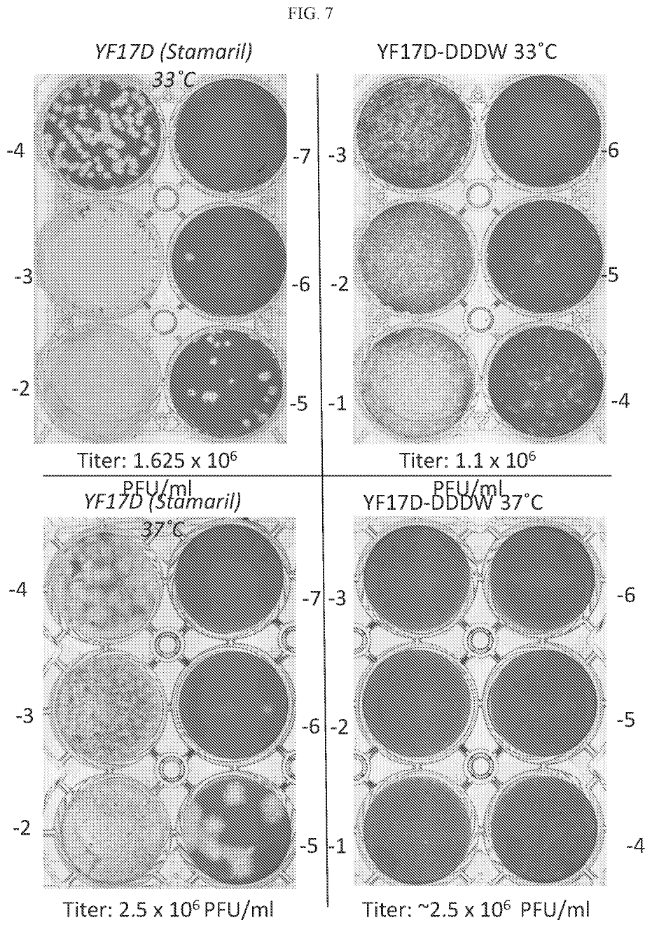

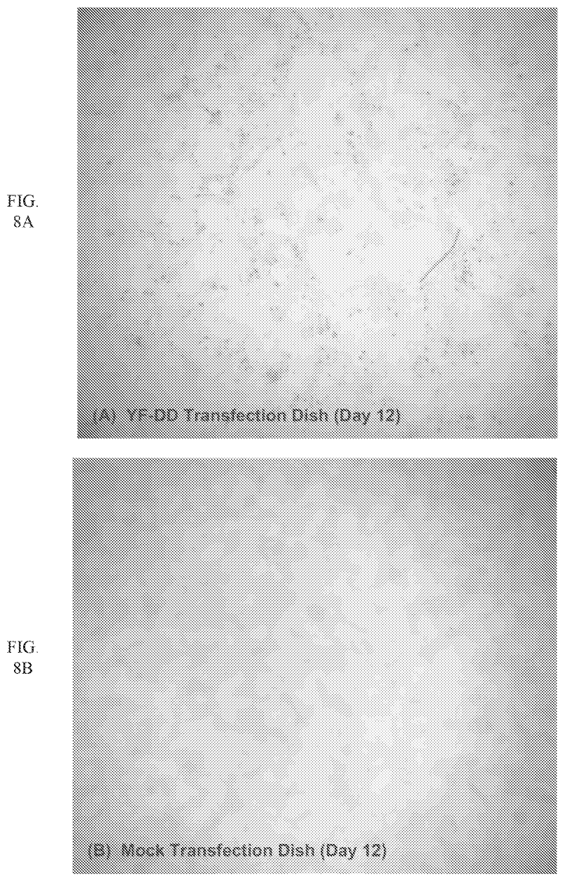

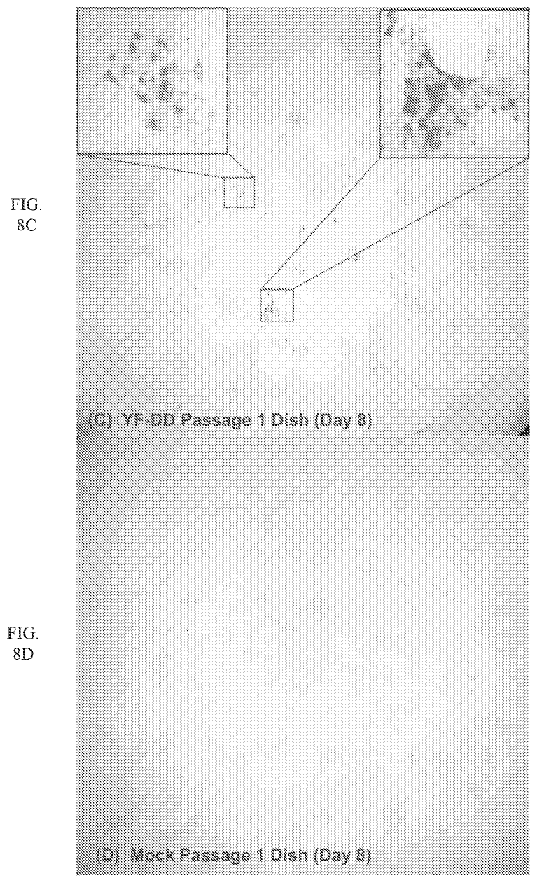

1. PCR for all 8 Fragment. All PCR reactions from original BioBasic plasmids were successful. All PCR products were purified by DNAland Gel Extraction PCR Purification 2-in-1 Kit. See FIG. 3A.
2. Overlapping PCR for Full Length YF-DD. Full length YF-DD (11 kb) was successfully generated by overlapping PCR. Full length products were further purified by DNAland Gel Extraction PCR Purification 2-in-1 Kit.
3. Diagnostic PCR Check. The first 8 diagnostic PCR check show correct pattern on both building block F2-DD (PCR product using in overlapping PCR) and full length YF-DD, indicating the first half of F2 region was correct deoptimized sequence without any WT contamination. The second sets of 8 diagnostic PCR showed correct pattern on both building block F2-DD (PCR product using in overlapping PCR) and full length YF-DD, indicating the second half of F2 region was the correct deoptimized sequence without any WT contamination.
4. RNA synthesis. Full length Overlapping PCR YF-DD were used in RNA synthesis. RNA was evaluated before transfection.
5. Detection of Yellow Fever Antigen by Immunohistochemical Staining of Transfected or Infected Cells. FIG. 8A-8D.

Yellow Fever Vaccine candidate YF-DD, which carries a wholly deoptimized E domain was successfully recovered by overlapping PCR and RNA transfection on Vero cells. Both the building block F2-DD and the full-length overlapping PCR products of YF-DD were PCR confirmed to carry the intended deoptimized DD sequence without detectable 17D sequence in the F2 region. Full length viral RNA was of high quality before transfection. The YF-DD virus was viable after transfection, as evidenced by a preponderance of infected cells upon immunohistochemical staining 12 days after RNA transfection.

YF-DD virus produced very little or no CPE after transfection. Blind passaging of the day 4 transfection harvest on fresh Vero cells confirmed the recovery of infectious YF-DD virus, as evidenced by a preponderance of newly infected cells upon immunohistochemical staining 8 days after infection (again without noticeable CPE). The absence of CPE is in stark contrast to the parental 17D virus under similar conditions (data not shown), indicating that YF-DD will likely be very highly attenuated.

The YF-DD virus will be further passaged, titered and sequenced to prepare it for mouse neurovirulence testing.

### Example 2

Assembly and Recovery of Yellow Fever 17D-WWDW from Synthetic DNA

### Experimental Overview

We recovered a rationally-designed, codon pair deoptimized (CPD) YFV vaccine candidate 17D-WWDW using our proprietary platform of codon-pair deoptimization technology, SAVE (Coleman et al., 2008, Mueller et al., 2010) based on sequences of the Yellow Fever vaccine strain 17D RK1 (Stock et al., 2012. GenBank: JN628279.1).

The full-length (FL) 17D-WWDW genome sequence was produced by overlapping PCR using a series of plasmid DNAs synthesized by BioBasic Inc. Eight viral genome fragments were amplified using YFV specific primers from corresponding plasmid DNA and reconstructed into a 10.862 kb full-length viral genome through overlapping PCR. Live 17D-WWDW virus was recovered following in vitro transcription and RNA transfection of Vero 10-87 WHO cells (cGMP manufactured master cell bank). 17D-WWDW was characterized by plaque phenotype (plaque assay) and sequence (Sanger sequencing of the CPD region).

### Experimental Procedures:

List of all reagents used for this project

| | Catalog | |
|---|---|---|
| Name of Reagent | # | Lot # |
| 2 × Q5 Master Mix | M0492L | 10072059 |
| HiScribe ™ T7 RNA Polymerase Mix | E2040S | 10087151 |
| ^{m7}G(5′) ppp(5′)A Cap Analog | S1405S | 10058022 |
| Lipofectamine ™ 2000-CD | 12566014 | 2196185 |
| OptiPRO SFM | 12309019 | 2187167 |
| MEM | 11095080 | 2185755 |
| DMEM | 11095080 | 2177997 |
| Penicillin/Streptomycin | 15140122 | 2199840 |
| Gibco FBS, Qualified | 10437036 | 1982167 |
| SuperScript ™ IV First-Strand | 18091050 | 01048694 |
| Synthesis | | |
| WHO 10-87 Vero cells R&D | | 1-101720-1 |
| WCB (Passage #15) | | |
| DNALand Gel Extraction Kit | GP1002 | SSGB1-1, |
| | | SSGBE-2, |
| | | SSEBH18 |
| PCR purification kit | T1020S | 10029809 |

### Genome Sequences

Original yellow fever virus strain 17D RK1 sequences were derived from GenBank JN628279.1.

### DNA Fragment Synthesis

To re-construct viral genomes de novo, the parental 17D genome was divided into eight DNA fragments and synthesized at BioBasic Inc. (Ontario, Canada). Fragment 2 contained the CPD sequences.

### PCR and Overlapping PCR

Amplification of eight (8) viral genome fragments from DNA plasmids followed standard PCR protocols. Q5® Hot Start High-Fidelity DNA Polymerase (NEB, Ipswich, Massachusetts) was used in all PCR processes to avoid introduction of spurious mutations.

To amplify genome fragments from DNA plasmids, a standard PCR setup was used. The 25 μl reaction contained 1 μl plasmid DNA, 1 μl of forward and reverse primers (detailed in Table 2) at 5 μM, 12.5 μl of the 2×Q5 Master Mix (MM) and 9.5 μl dH2O. Reaction parameters were as follows: 98° C. 120 sec to initiate the reaction, followed by 27 cycles of 98° C. for 10 sec, 60° C. for 15 sec, and 72° C. for 60 sec and a final extension at 72° C. for 7 min. Amplicons were verified by agarose gel electrophoresis and gel purified using the DNALand Gel Extraction Kit (DNALand Scientific). All PCR fragments were eluted with

30 μl dH₂O and final concentrations of purified DNA fragments were quantified by Nanodrop.

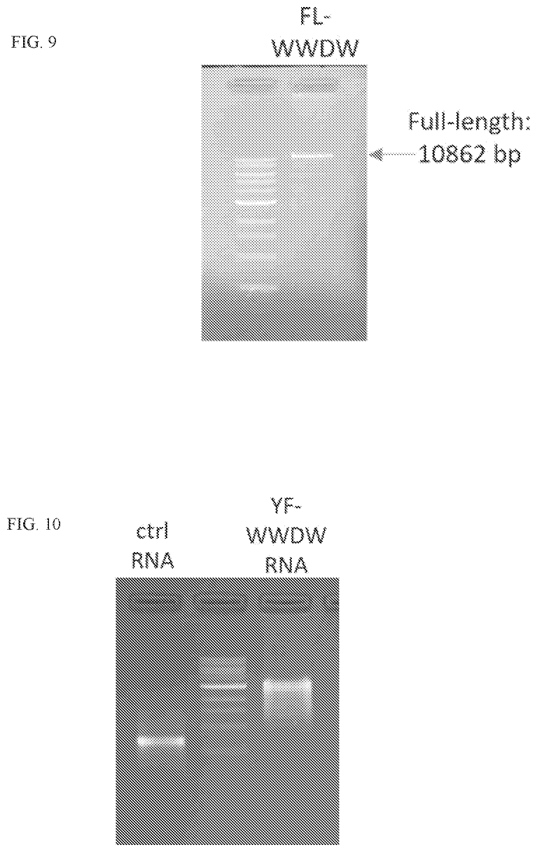

For overlapping PCR, a mix of 40 ng of each DNA fragment (keeping the molar ratio at 1:1), 10 μl 2×Q5 MM and dH₂O to a final volume of 20 μl was made. This first PCR reaction was carried out under the following conditions: 98° C. for 30 sec, and 65° C. for 6 min 30 sec for 10 cycles. After the initial extension, 2 μl of the first PCR reaction was added into a new tube containing 1 μl of the 5′ and 3′ flanking primers (5 μM) for whole genome, 10 μl of the 2×Q5 master mixture, and 10 μl dH₂0, and mixed well. This second PCR reaction was performed under the following conditions: 98° C. for 10 sec, 60° C. for 45 sec, and 65° C. for 6.5 min, for 15 cycles. The final 10.862 kb product was visualized on a 0.9% agarose gel (FIG. 9).

### In Vitro Transcription

Overlapping PCR products containing the full-length 17D-WWDW viral genome were used directly for capped in vitro transcription using the HiScribe™ T7 In Vitro Transcription Kit. Briefly, 4 μl of overlapping PCR product was mixed with 2 μl 1 OX reaction buffer, 2 μl of GTP, UTP, CTP (each at 100 mM concentration) and 2 μl of ATP (20 mM), 4 μl 40 mM m7G(5′)ppp(5′)A RNA Cap Structure Analog (NEB) and 2 μl of T7 RNA polymerase mix. The reaction was incubated at 37° C. for 2.5 hrs. 1 μl of the in vitro transcription product was subjected to gel electrophoresis to assess RNA quality and quantity. RNA was used immediately for transfection. The remaining RNA was purified by LiCl method (Ambion) and stored at −80° C.

### RNA Transfection

RNA transfection was performed using Lipofectamine® 2000 CD following manufacturer's instructions. Vero cells were seeded into 12-well plates and allowed to grow to near 80-85% confluence. For transfection, 0.2-2 μl Lipofectamine2000 CD and 0.1-1 μl RNA (keeping the ratio of Lipo:RNA=2:1) were diluted in 100 μl OptiPRO. 3-4 hrs later, transfection medium was exchanged for 2 ml OptiPRO+DMEM (v:v=50%:50%). Plates were monitored daily for cytopathic effect (CPE). Supernatant was collected when CPE appeared (3-4 days post transfection). Supernatants were stored at −80° C. for analysis of virus titers and virus passage.

### Virus Passaging

Viral supernatant collected following the RNA transfection was diluted to 1:1000. 10 μl of this viral dilution were added to a 35-mm dish of Vero cells (85-90% confluence) containing 800 μl OptiPro medium for infection in a 33° C. incubator. 3.5 hrs later, the infection medium was removed and 3 ml of OptiPRO+DMEM (v:v=50%:50%) were added and the dish continued to incubate in a 33° C. incubator. Cells were monitored daily for cytopathic effect (CPE). Virus harvested from passage 2 was processed for Sanger sequencing (described below).

### Virus Growth Kinetics

To test virus growth kinetics, Vero cells were seeded in a 6-well plate and allowed to reach near confluency. Cells were then infected with the parental 17D and the deoptimized 17D-WWDW at an MOI=0.01. Specifically, medium was replaced with 800 ul OptiPRO SFM in each well. After adding the virus stock at proper dilution, plate was gently rocked at RT for 30 min. Then, the plate was placed in a 33° C. cell culture incubator for 2.5 hr. At the end of the incubation, inoculum was aspirated and 2 ml fresh OptiPRO+DMEM (v:v=50%:50%) were added to each well. Cells were further incubated for 3 days or until cytopathic effect became widespread. Supernatants were collected daily for plaque assay (PA) to quantify viral titers.

### RT-PCR

Viral RNA was isolated from the cell culture supernatant using Viral RNA isolation kit from Qiagen (Germantown, MD). Reverse transcription was carried out using SuperScript™ IV First-Strand Synthesis System with random hexamers and a 3′-end of genome-matched specific primer #2534 to increase the chances of getting a complete 3′ end. This viral RNA was used to set up sequencing reactions for the full-length viral genome to examine the genetic stability of the genome when CPD sequences were introduced.

### Plaque Assay (PA)

To characterize the growth properties of the parental 17D and 17D-WWDW viruses, a plaque assay was performed in Vero cells. Cells were seeded in 12-well plate and allowed to grow to ˜80-90% confluence. Ten-fold serial dilutions (from 10-2-10-7) of viruses were made in OptiPRO.

Cells were incubated with 400 μl/well (12-well) of virus dilution for 3 hours at 33° C. or 37° C. After virus adsorption, 1.6 ml/well (for 12-well plate) of agarose overlay was added to each well. The agarose overlay was prepared as following: 20 ml 1% LMP agarose in MEM (final: 0.8%, freshly made); 0.5 ml FBS (final: 2%); 0.25 ml 100× Penicillin/Streptomycin (final: 1×; Invitrogen)

The plates were incubated at 37° C. or 33° C. for 5 days. Each well was fixed by the addition of 2 ml/well of 10% formaldehyde for 1-2 hr at RT. The agarose overlay was removed, and the cells were stained with crystal violet to visualize the plaques.

### Full-Length Genome Sequencing and Data Analysis

17D-WWDW virus harvested at passage 2 (lot 9-073021-1-3) from WHO 10-87 WCB Vero cells was sequenced. cDNA was synthesized from the viral RNA and used to amplify genome segments by standard PCR using primers listed in Table 2 except 2519F was replaced with 2557F (AGT AAA TCC TGT GTG CTA ATT GAG GTG (SEQ ID NO:17), starting from authentic 5′-end of YFV genome) for fragment 1. Sequencing was performed by Genewiz Inc (South Plainfield, NJ). Samples were prepared per company's guidance (sequencing tracking #: 30-563313543 and 30-564734213). Sequencing results were analyzed using online tool NCBI Blastn.

### Results

Features of 17D-WWDW after Codon Pair Deoptimized Design

We targeted the coding sequence of YF glycoprotein Envelope (E) gene, which functions in viral attachment, entry, and membrane fusion, for codon-pair deoptimization. The schematic of the YFV genome (FIG. 1) shows the polyprotein coding region and the coding regions of polypeptides before proteolytic processing. The structural E region was recoded in CPD manner. In the schematic, D stands for the region with CPD sequence (D, red) and W is YF-17D wild-type (W, blue) sequences. The number of nucleotides changed in CPD within E gene is shown in the table of FIG. 1.

### Generation of the Full-Length 17D-WWDW Genome DNA by Overlapping PCR

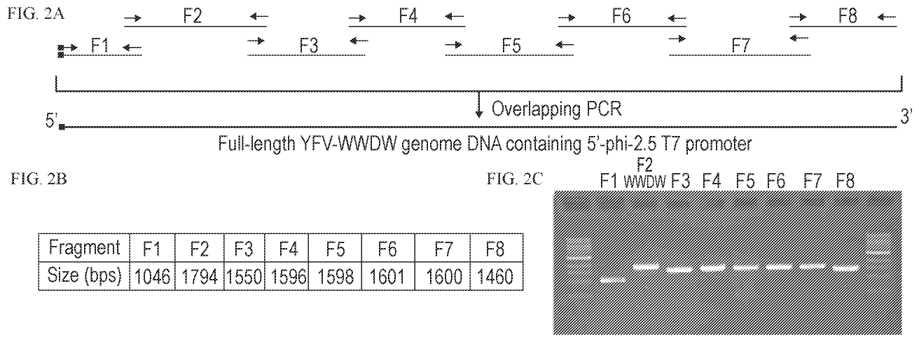

To generate the full-length 17D-WWDW genome DNA from chemically synthesized DNA plasmids, eight genome fragments (sized from 1.0-1.8 kb) were amplified from eight individual plasmids containing different genome fragments of 17D-WWDW, as indicated in FIG. 2. Oligo nucleotide primers were ordered on 7/10/2021 and received on Jul. 14, 2021. Full-length 17D-WWDW genome DNA was then successfully generated by overlapping PCR of eight fragments as shown in FIG. 9. Fragment 2 (F2) contains the CPD sequences.

Generation of the Full-Length 17D-WWDW RNA by In Vitro Transcription and Transfection into Vero Cells

The full-length 17D-WWDW PCR template was used for RNA transcription. RNA integrity was verified by running 1 ul of in vitro transcript on 0.6% TAE-Agarose gel (FIG. 10). The results confirmed that the full-length 17D-WWDW RNA was successfully transcribed. 1 μl of the 17D-WWDW RNA was used for RNA transfection into WHO 10-87 Vero cells (˜80% confluence, OptiPro serum-free medium) by using Lipofectamine2000®-CD reagent as described in Experimental Procedures. Cells were checked daily for CPE. CPE was observed on day 3 post-transfection. It took approximately 10 days to recover live 17D-WWDW from the date of receipt of PCR primers.

Identification of the Deoptimized Region of F2-WWDW by Two-Step Procedure after Recovery of Viable 17D-WWDW Virus

When viable 17D-WWDW virus was detected on day 3 post transfection, supernatants were collected from infected Vero cells and viral RNA was extracted by using QIAamp Viral RNA kit. RT-PCR was used to produce viral cDNA by using SuperScript™ IV First-Strand Synthesis System. To verify the 17D-WWDW virus generated from overlapping PCR contained no wild-type sequences in F2 and that the deoptimized sequence did not accumulate spurious mutations, we checked the genomes of newly recovered 17D-DW by plaque assay at passage 1 and Sanger sequencing at passage 2.

### Plaque Assay at 37° C. and 33° C.

If the 17D-WWDW virus was contaminated with parental 17D virus, the plaque assay would show two different plaque phenotypes in the same well. The following figures demonstrated that the 17D-WWDW virus recovered from overlapping PCR template was a uniform culture of deoptimized virus without contamination of parental 17D incubated at 37° C. for 5 days (FIG. 11).

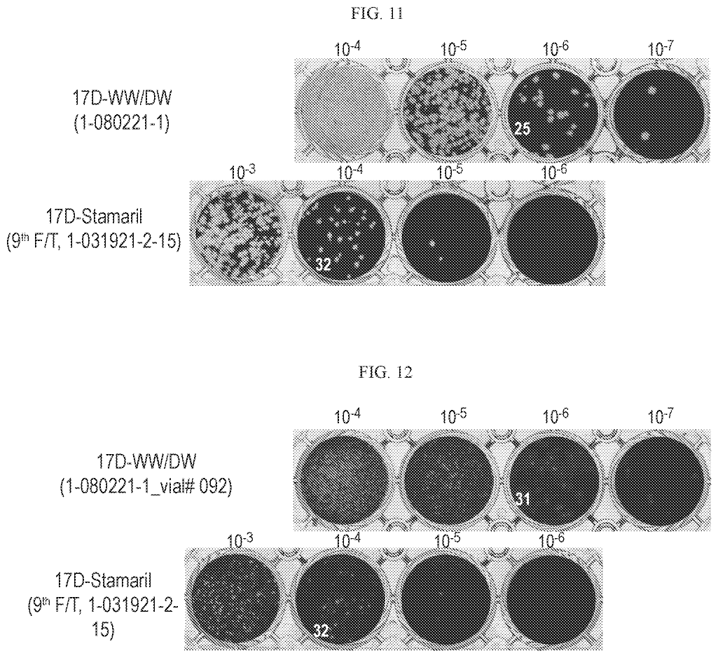

When the plaque assay was carried out at 33° C., 17D-WWDW plaques appeared smaller and less crisp than at 37° C. (FIG. 12). The plaque sizes from both 17D-WWDW and parental 17D at 33° C. were smaller than those observed at 37° C., but plaques from 17D-WWDW were more diffuse and less open than parental 17D (FIG. 12).

### Sanger Sequencing

The 17D-WWDW virus recovered at passage 2 (D3, ID: 9-073021-1-3) was prepared for whole genome sequencing. Results of DNA sequencing demonstrated that 17D-WWDW F2 fragment carries all deoptimized sequences as originally designed when aligned to the reference 17D sequences.

### Discussion and Conclusions

Live attenuated, codon pair deoptimized vaccine candidate against yellow fever, 17D-WWDW, was successfully recovered following genome reconstruction via overlapping PCR and transfection. From the time of receipt of PCR primers, it took approximately 10 days to recover live 17D-WWDW. Two different methods verified that the recovered 17D-WWDW viral genome was correct as designed and did not accumulate any spurious mutations in the deoptimized region. Whole genome sequencing of the passage 2 virus demonstrated that the sequence of the vaccine candidate was as designed and did not contain spurious mutations.

Plaque morphology of 17D-WWDW and parental 17D were similar at 37° C.: crisp, large, well-defined plaques. 17D-WWDW exhibited an altered plaque phenotype at 33° C. as compared to parental 17D, forming plaques that were more diffuse and less open than 17D. This suggests that 17D-WWDW may have a temperature sensitive phenotype as compared to 17D.

### Example 3

### Full-Length Sequencing of Yellow Fever 17D-WWDW at Passages 5, 12, and 15

The full-length sequencing of 17D-WWDW vaccine candidate at passage 5 (ID: 9-100321-1), passage 12 (ID: 9-013020-1), and passage 15 (ID: 9-020822-1) is described herein.

To demonstrate sequence stability and define a sequence for yellow fever (YF) vaccine candidate 17D-WWDW vaccine virus was passaged in cell culture for 15 passages post-recovery and sequenced at passage 5, 12 and 15 using whole genome Sanger sequencing. Specifically, standard RT-PCR was performed, and 8 fragments were PCR-amplified and subjected to Sanger sequencing to confirm virus identity and to identify any spurious mutations. Sequencing reactions were prepared and sequenced. The resulting sequence was aligned with the designed 17D-WWDW sequence.

No nucleotide mutations were detected in the YF-WWDW genome at any passaged sequenced up to and including passage 15.

During passage of 17D-WWDW in Vero cell culture we detected no point mutation from passage 1 to passage 15. Viral genome at passage 15 was 100% identical at the nucleotide level to the designed sequence, indicating that 17D-WWDW was genetically stable for 15 passages in cell culture.

### Experimental Procedures

### Cells

Vero WHO 10-87 R&D working cell bank (Passage 8)

### Medium and Reagents Used

| Name of Reagent | Manufacturer | Catalog # | Lot # |
|---|---|---|---|
| 2 × Q5 MasterMix | New England Biolabs | M0492L | 10072059 |
| OptiPRO SFM | Thermo Fisher Scientific | 12309019 | 2187167 |
| SuperScript ™ IV | Thermo Fisher Scientific | 18091050 | 01048694 |
| Trizol Reagent | Ambion | 15596026 | 263912 |
| PCR purification kit | New England Biolabs | T1020S | 10029809 |

Step 1: Viral passage. Viral supernatant was collected on day 4 following the RNA transfection. 1 μl of this viral dilution was added to a 35-mm dish of Vero cells (85-90% confluence) containing 800 μl OptiPro medium for infection in a 33° C. incubator and incubated for 3.5 hrs. The infection medium was removed and 3 ml of OptiPRO+DMEM (v:v=50%:50%) were added and the dish continued to incubate in a 33° C. incubator. Cells were monitored daily for cytopathic effect (CPE). Approximately 3 days were required to develop ˜30-40% CPE. Then, virus supernatant was harvested. Virus collected from passages 5, 12 and 15 was processed for Sanger sequencing.

Step 2: Viral RRNA extraction and Viral cDNA synthesis. Viral RNA was isolated from the cell culture supernatant using Viral RNA isolation kit from Qiagen (Germantown, MD). Reverse transcription was carried out using SuperScript™ IV First-Strand Synthesis System with random hexamers and a 3′-end of genome-matched specific primer #2534 to increase the chances of getting a complete 3′ end.

1 μl of viral RNA (vRNA) was used as a template for cDNA synthesis using SuperScript IV First-Strand cDNA synthesis kit.

Specifically, 0.5 μl Random Hexamer, 0.5 μl 10 mM dNTP, 1 μl vRNA, 4.5 μl H₂O were mixed and heated at 65° C. for 5 min, then immediately incubated on ice for 2 min. The reaction was mixed with 0.5 μl RNase Inhibitor, 2 μl 5×SSIV buffer, 0.5 μl 100 mM DTT, and 0.5 μl SuperScript IV Reverse Transcriptase (200 U). The reaction was then incubated at 23° C. for 10 min, followed by 50° C. for 40 min and 80° C. for 10 min.

Step 3: PCR to generate fragments. NEB 2×Q5 MasterMix (MM) system was used to generate eight 1.8 kb fragments of YF-WWDW vaccine candidate (CDX-460) by standard PCR using primers listed in Table 2 except Primer #2519 was replaced with 2557F (AGT AAA TCC TGT GTG CTA ATT GAG GTG (SEQ ID NO: 17)), starting from authentic 5′-end of YFV genome) for fragment 1 at passage 5 and 8, respectively.

In each reaction, 0.5 μl of freshly made cDNA was used as template, mixed with 1 μl of 1 μM Forward primer, 1 μl of 1 μM Reverse primer, 2.5 μl H₂O, and 5 μl NEB 2×Q5 MM.

17D-WWDW vaccine candidate RT-PCR fragments from Passages 5, 12, and 15 are shown in FIG. 3B.

### Example 4

Full-Length Sequencing of Yellow Fever 17D-WWDW from Non-Human Primate Sera

### Experimental Overview

To demonstrate sequence stability of YF vaccine candidate 17D-WWDW after replication in vivo, virus isolated from a serum sample from Southern Research NHP study 16128.02 was sequenced. Samples from this study were shipped frozen. A sample of serum taken from animal 18164 at 4 days post-vaccination with 1×10⁵ PFU of 17D-WWDW was used for genome isolation and Sanger sequencing. The viral RNA was extracted, standard RT-PCR was performed, and 8 fragments were PCR-amplified and subjected to Sanger sequencing to confirm virus identity and to identify any spurious mutations.

Sequencing samples were prepared under BSL2 containment and submitted to Genewiz and Eurofins for sequencing. The resulting sequence was aligned with the designed sequence of the designed 17D-WWDW on the backbone of the YF vaccine strain 17D.

No nucleotide mutations were detected in the viral genome RNA extracted from the specified serum sample. 17D-WWDW remained identical to the design sequence following replication in vivo.

### Experimental Procedures

### Reagents and Kits Used

| Name of Reagent | Manufacturer | Catalog # | Lot # |
|---|---|---|---|
| QIAamp Viral RNA Kit | Qiagen | 52906 | 166012744 |
| 2 × Q5 MasterMix | New England | M0492L | 10072059 |
| | Biolabs | | |
| SuperScript ™ IV | Thermo Fisher | 18091050 | 01048694 |
| | Scientific | | |
| PCR purification kit | New England | T1020S | 10029809 |
| | Biolabs | | |

Step 1: Viral RNA extraction and viral cDNA synthesis: Viral RNA was isolated from 140 μl NHP serum using QIAamp Viral RNA Kit from Qiagen (Germantown, MD). Reverse transcription was carried out using SuperScript™ IV First-Strand Synthesis System with random hexamers and a 3′-end of genome-matched specific primer #2534 to increase the chances of getting a complete 3′ end. 9.5 μl of viral RNA (vRNA) was used as a template for cDNA synthesis using SuperScript IV First-Strand cDNA synthesis kit.

Specifically, 1 μl Random Hexamer, 1.5 μl specific primer #2534, 1 μl 10 mM dNTP, 9.5 μl vRNA were mixed and heated at 65° C. for 5 min, then immediately incubated on ice for 2 min. The reaction was mixed with 1 μl RNase Inhibitor, 1 μl 100 mM DTT, 4 μl 5×SSIV buffer, and 1 μl SuperScript IV Reverse Transcriptase (200 U). The reaction was then incubated at 23° C. for 10 min, followed by 50° C. for 40 min and 80° C. for 10 min.

Step 2: PCR to generate fragments: NEB 2×Q5 MasterMix (MM) system was used to generate eight 1-1.8 kb fragments of YF-WWDW vaccine candidate (CDX-460) by standard PCR using primers.

In each reaction, 1 μl of freshly made cDNA was used as template, mixed with 1 μl of 2.5 μM Forward primer, 1 μl of 2.5 μM Reverse primer, 4.5 μl H₂O, and 10 μl NEB 2×Q5 MM.

Step 3: Full-length genome sequencing and data analysis: 0.5-2 μl of each PCR fragment generated in Step 2 was used directly in sequencing reactions or, if any primer dimers were found on 1% agarose gel, were column purified by NEB cleanup PCR purification kit. Sequencing reactions consisted of 18-20 ng unpurified/purified PCR product, 2 μl of 5 μM sequencing primers, added to a total volume of 15 μl. The sequencing sample tubes were submitted to Genewiz Inc (South Plainfield, NJ) and Eurofins (Louisville, KY) for Sanger sequencing. Samples were prepared per company's guidance. Sequencing results were analyzed using NCBI Blastn online tool.

### Results

17D-WWDW vaccine candidate RT-PCR fragments from viremia sample #18164 are shown in FIG. 3C.

There were no point mutations in 17D-WWDW viral genome from viremia sample #18164

After amplification of the viral genome sequence from viremia sample #18164, no mutations were detected in the entire viral genome (17D-WWDW).

### Discussion and Conclusions

We successfully extracted the viral RNA from the serum sample of a non-human primate (ID; #18164, 4 days post-vaccination, SR study 16128.02) that had a very low viral (˜331 PFU/ml verified by conversion of qRT-PCR assay, see report KP01). The genome fragments were amplified by a conventional RT-PCR approach. The result of full-length genome sequencing demonstrated 17D-WWDW remained 100% identical to the designed sequence following replication in vivo, demonstrating genetic stability.

### Example 5

### Scalable, Genetically Stable and Safe Live Attenuated Vaccine Against Yellow Fever Virus

Materials and Methods. As noted in the above examples, the sequence of 17D-204 vaccine strain YF/Vaccine/USA/Sanofi-Pasteur-17D-204/UF795AA/YFVax was derived from GenBank entry JX503529. The E gene sequence was subjected to the SAVE algorithm to design two vaccine candidates with varying extents of deoptimization. 17D-WWDW carries deoptimizing mutations over approximately 25% of the length of the gene and contains 88 synonymous mutations. 17D-WD carries mutations nearly 50% of the E gene and contains 171 mutations. The deoptimized 17D-WWDW and 17D-WD E gene fragments were synthesized de novo. Synthetic deoptimized fragment was assembled with several other 17D gene fragments using overlapping PCR to generate full-length 17D-WD and 17D-WWDW. Full length RNA was then in vitro transcribed from the template and transfected into WHO 10-87 Vero cells and live virus was recovered and characterized in vitro. Virus was passaged for 15 passages in Vero cells to demonstrate genetic stability.

Safety and Immunogenicity in Non-human Primates (NHPs): Rhesus macaques, six males and six females, 1-4 years of age, were randomized into four groups (n=3). On Days 0 and 30, animals were vaccinated by the subcutaneous (SC) route with 17D-WD (10⁶ PFU) or two dose levels of 17D-WWDW (105 and 10⁷ PFU) or control YF 17D Reference Vaccine 168-73 (10⁵ PFU). Daily clinical observations along with monitoring of animals' body weight, body temperature, and food consumption were performed daily. Blood samples were collected on Days 0, 2, 4, 6, 30, 32, 34, 36 for viral load assessment (via qRT-PCR) and on Days 0, 21, 30, and 51 for immunogenicity assessment (via plaque reduction neutralization assay (PRNT) against reference virus 17D).

### Results

Two deoptimized vaccine candidates, 17D-WD and 17D-WWDW, based on the 17D strain of Yellow Fever were successfully recovered and sequence confirmed. Both vaccine candidates were well tolerated in NHPs following SC administration. Injection sites appeared normal post primary and boost immunizations and none of the animals showed cutaneous reactions. All clinical observations were normal for animals receiving the two deoptimized vaccines and 17D vaccine. There was no significant weight loss or fever observed in any of the animals in any group during the study.

Serum collected on days 21 and 30 showed an average 30- to 200-fold higher antibody level as compared to baseline titers at day 0. This increase in PRNT endpoint titers in day 51 samples (post-boost) went as high as 300- to 800-fold higher than baseline, indicating that boosting was beneficial in increasing antibody titers for all vaccines tested in the study. After both prime and boosting dose, candidate vaccine 17D-WWDW (at both 10⁷ and 10⁵ dose levels) produced levels of nAbs that were not statistically different from 17D at 10⁵ PFU after either the prime or the boosting dose (p-values >0.05). Levels of nAbs produced by candidate vaccine 17D-WD after the first dose were significantly lower than those of the 17D comparator (p=0.0001 at day 30). As a result of providing a stronger boost in antibody response than 17D, 17D-WD titers rose after the second dose to levels not statistically different from those seen after 2 doses of 17D (p>0.05).

Viremia was detected in all animals on Day 2 at a low titer, which decreased and/or became undetectable in most animals by day 6. 17D-WD and 17D-WWDW vaccinated animals had overall fewer days of viremia than 17D vaccinated animals. There was a trend of 17D-WD viremia dropping more rapidly, becoming undetectable in all animals on d6 post vaccination, suggesting that 17D-WD maybe be more attenuated than the 17D reference vaccine. However due to the small group sizes and spread of the values within the groups neither group mean viremia titers reached statistical significance relative to the reference vaccine group (p>0.05).

There was no detectable viremia in any animal on Days 32, 34, and 36 (2, 4, and 6 days after dose 2), suggesting that vaccinated animals were protected from the surrogate challenge posed by the vaccine booster dose.

Additional details of this study and data are described in the example below.

### Example 6

### Safety and Immunogenicity of a Live Attenuated Yellow Fever (YF) Vaccine in Rhesus Macaques

The objective of this non-GLP study was to evaluate the safety, attenuation and immunogenicity of two live attenuated yellow fever (YF) vaccine candidates (YF 17D-WD and 17D-WWDW) as compared to YF 17D reference vaccine in Rhesus Macaques when administered subcutaneously using a primary and boost vaccination approach.

### Test Articles

| | |
|---|---|
| Name: | YF (Groups 1, 2 and 3) |
| Viral | 17D-WD - 3 × 10⁶ PFU/mL |
| Titer: | 17D-WWDW - 6.25 × 10⁷ PFU/mL |
| Character- | Codon-pair deoptimized live-attenuated yellow fever vaccines |
| ization: | based on the 17D vaccine strain. Additional information can |
| | be found in the certificate of analysis. |
| Formula- | The vaccine candidates were diluted for Day 0 and Day 30 |
| tions: | vaccinations as shown in Group Assignments. |
| Routes: | Test articles were administered via the SC route on Days 0 |
| | and 30. |
| Storage: | The vaccines were stored at −80° C. ± 10° C. until diluted |
| | for vaccination. Vaccines were stored on wet ice during |
| | transfer and dose administration. |

### Vaccine Diluent

| | |
|---|---|
| Name: | 50/50 DMEM and OptiPro |
| Special Handling: | Aseptic technique |
| Storage: | 2-6° C. |

### Control Article

| | |
|---|---|
| Name: | YF 17D Reference Vaccine Provided by NIBSC |
| Lot | 168-73 |
| Number(s): | |
| Manu- | Prepared by Robert Koch Institute and supplied by |
| facturer: | NIBSC |
| Special | This reagent was handled under BSL-2/ABSL-2 conditions. |
| Handling: | Personnel wore surgical protective gloves and avoided |
| | contact with skin, eyes, or mucous membranes when |
| | handling the reagent. |
| Titer: | Actual titer (in log10 PFU) was documented in the study |
| | records. Animals were dosed with a target dose of 5 × |
| | log10 PFU/animal. |
| Routes: | Control article was administered via the SC route on Days |
| | 0 and 30. |
| Character- | The titer on the label indicated 5 × 10⁵ PFU/ampule |
| ization: | and passage level for this lot was #237. |
| Stability & | Lyophilized material. On receipt it was stored in the |
| Storage: | freezer (−20° C. or below). |

### Reference Vaccine Diluent

| | |
|---|---|
| Name: | Water for Injection (WFI) |
| Special | Aseptic technique. |
| Handling: | |
| Character- | No DNase, RNase, or protease activity detected. |
| ization: | Compliance with current USP monograph test |
| | requirements for Water for Injection (WFI). |
| Stability & | Room Temperature (15-30° C.). |
| Storage: | |

### Test System

| | |
|---|---|
| Species & Strain: | Rhesus Macaques (Macaca mulatta) |
| Supplier: | Documented in the study records |
| Quarantine: | At least 35 days (the actual duration is documented |
| | in the study records) |
| Age at Study | 1-4 years of age (Actual ages were documented in |
| Start: | the study records) |
| Weight at Study | 3.5-6.0 kg (Actual weights were documented in the |
| Start: | study records) |
| Number on Study: | 12 |
| Sex: | 6 males and 6 females |

On 20 Jul. 2021, a total of twelve Rhesus macaques (Macaca fascicularis) (six males and six females) were received at Southern Research (SR) in Birmingham (AL) from SR location in Frederick (MD) and quarantined in the A/BSL-2 facility for 27 days prior to study initiation. The animals were quarantined and observed at the SR Frederick site before re-location. The animals were uniquely identified by tattoo.

All animals were observed twice daily throughout the quarantine and study periods for signs of morbidity and mortality.

On the first day of dosing with the test article (17 Aug. 2021), the animals were 2.8-4.1 years old and weighed 4.4-5.3 kg (males) and 3.1-4.4 kg (females). Animals were single housed in stainless steel cages in an environmentally monitored, ventilated room at Southern Research (SR). Fluorescent lighting provided illumination approximately 12 hours per day. Animals were fed commercial 2025C Primate Diet (Envigo, Madison, WI) twice daily during the quarantine and throughout the study.

Water from Birmingham's public water supply was provided ad libitum during the quarantine and study periods via an automatic watering system. Analyses of the feed, provided by the manufacturer, and the analyses of periodic water samples were reviewed by Southern Research's Veterinarian, or designee, to assure that no known contaminants were present at levels that could interfere with and affect the outcome of the study. Animals were provided with consumable enrichment and toys.

Housing and animal care conformed to the guidelines of the U.S. Department of Agriculture, the Guide for the Care and Use of Laboratory Animals, and to the applicable Standard Operating Procedures (SOPs) of Southern Research.

### Study Design Overview

This study was designed to assess the safety and immunogenicity of two live attenuated yellow fever (YF) vaccine candidates (YF 17D-WD and 17D-WWDW) in Rhesus macaques. Prior to Day 0, twelve (12) rhesus macaques (six males and six females) were randomized into four groups (three animals per group, mixed sex) according to gender/weight using Provantis Software. Two males and one female were randomized into both Group 1 and Group 3. One male and two females were randomized into both Group 2 and Group 4. On Days 0 and 30, animals were vaccinated via SC injection with YF Vaccines 17D-WD (10⁶ PFU) or two dose levels of 17D-WWDW [10⁵ PFU (low) and 10⁷ PFU (high)] assigned to Groups 1, 2, and 3, respectively, or control YF 17D Reference Vaccine (Group 4, 10⁵ PFU) as outlined in Group Assignments.

Clinical signs, injection site monitoring, food consumption, body weight, and body temperature were assessed as indicated in Study Schedule. Blood was collected on Days 0, 2, 4, 6, 21, 30, 32, 34, 36, and 51. Study termination occurred on Day 51 (animals were not euthanized).

| | | | Test Article/Control Article Administration | | | | | Study |
|---|---|---|---|---|---|---|---|---|
| | Number of | Animal | | Volume | Dose | | | Termination |
| Group | Animals | IDs | Name | (mL) | (PFU) | Frequency | ROA 1 | Day |
| 1 | 3 (2M/1F) | 18160 (M) | 17D - | 0.5 | 106 | Day 0, | SC | 51 |
| | | 18155 (M) | WD | | | Day 30 | | |
| | | 18161 (F) | | | | | | |
| 2 | 3 (1M/2F) | 18159 (M) | 17D - | 0.5 | 105 | Day 0, | SC | 51 |
| | | 18166 (F) | WWDW | | | Day 30 | | |
| | | 18164 (F) | | | | | | |
| 3 | 3 (2M/1F) | 18157 (M) | 17D - | 0.5 | 107 | Day 0, | SC | 51 |
| | | 18158 (M) | WWDW | | | Day 30 | | |
| | | 18162 (F) | | | | | | |
| 4 | 3 (1M/2F) | 18156 (M) | YF 17D 168- | 0.5 | 105 | Day 0, | SC | 51 |
| | | 18165 (F) | 73 | | | Day 30 | | |
| | | 18163 (F) | | | | | | |

1 Route of Administration

### Study Schedule

| Study Day | 0 | 2 | 4 | 6 | 7 | 14 | 21 | 23 | 25 | 27 | 30 | 32 | 34 | 36 | 51 |
|---|---|---|---|---|---|---|---|---|---|---|---|---|---|---|---|
| Vaccination | x | | | | | | | | | | x | | | | |
| Daily Mortality Check | Animals were observed twice daily (AM/PM) | | | | | | | | | | | | | | |
| Weight, Temp, clinical signs with | Daily Days | | | | | | | | | | Daily Days | | | | |
| clinical score, food consumption¹ | 0-6 | | | | | | | | | | 30-36 | | | | |
| Injection site monitoring | x | x | x | x | | | | | | | x | x | x | x | x |
| Blood: Serum for PRNT2 (2.0 mL | x | | | | | | x | | | | x | | | | x |
| SST3) | | | | | | | | | | | | | | | |
| Blood: Serum (viremia2) (2.0 mL | x | x | x | x | | | | | | | x | x | x | x | |
| SST3) | | | | | | | | | | | | | | | |
| End of study (animals were not | | | | | | | | | | | | | | | x |
| euthanized) | | | | | | | | | | | | | | | |

¹Body weight and temperature data were collected during blood sample collection when possible to avoid frequent anesthesia.
²Samples were shipped for analysis following Day 21 collection and again after study termination.
³SST—Serum Separator Tube

### Vaccine Administration

On Days 0 and 30, 12 animals (all groups) were immunized subcutaneously via inoculation with a single dose of a test article or the reference vaccine in the right or left quadriceps, respectively, as outlined for each group in Group Assignments. Prior to vaccination, the hair was removed from the injection site, the site was marked with an indelible marker and cleaned with alcohol.

### Clinical Observations

All animals were observed for signs of mortality and morbidity twice daily (AM/PM) during quarantine and study periods. Detailed clinical observations were performed daily on Days 0-6, and Days 30-36 as shown in Study Schedule. Each day, each animal was given a numerical score based on the scale as specified by the WHO technical report series (TRS) 978:

0—normal, clinical sign not present; 1—rough coat, not eating; 2—high-pitched voice, inactive, slow moving; 3—shaky movements, tremors, incoordination, limb weakness; 4—inability to stand, limb paralysis or death.

### Injection Site Monitoring

Injection sites were monitored daily on Days 0-6 and 30-36 as outlined in Study Schedule. Clinical signs including but not limited to rash, erythema, and swelling observed at the injection sites were recorded.

### Blood Collection

Animals were anesthetized and blood was collected into serum separator tubes (SST) to supply the volumes indicated in Study Schedule. Samples were taken from right femoral (RF) or left femoral (LF) vein.

### In-Life Parameters

For all animals, rectal temperatures and body weights were collected as outlined in Study Schedule.

### Food Consumption

Food consumption was monitored and recorded for all study animals as shown in Study Schedule.

### Anesthesia

Animals were anesthetized for the following procedures:
SC Vaccination
Blood collection
Detailed Clinical Observation
Temperature and body weight collections
Preparation for euthanasia
Cage changes (if needed)

Animals were anesthetized with Ketamine HCl at approximately 10-30 mg/kg via intramuscular (IM) injection. When additional anesthesia was required, the original substance was given at no more than ½ of the original dose for subsequent injections.

### In-Vitro Test Procedures

### Test Article Preparation

The vaccines were provided by the Sponsor. The test articles were kept at ≤−80° C. until the day of vaccination. Prior to vaccination, test articles were thawed at room temperature, diluted in diluent provided by the Sponsor, 50/50 DMEM and OptiPro, as indicated below and injected subcutaneously.

| | | | | Total | | | | |
|---|---|---|---|---|---|---|---|---|
| | | | | Inoculum | Diluent | Vaccine | | Volume/ |
| | Vaccine | Dose | | Volume | Volume | Volume | | animal |
| Group | Name | (PFU) | N + 1 | (mL) | (μL) | (μL) | | (mL) |
| 1 | 17D-WD | 10⁶ | 4 | 2 | 667 | 1333 | uL | 0.5 |
| 2 | 17D-WWDW | 10⁵ | 4 | 2 | 1980 | 20 μL of diluted | | 0.5 |
| | | | | | | WWDW in #31 | | |
| 3 | 17D-WWDW | 10⁷ | 4 | 2 | 1360 | 640 | uL | 0.5 |

¹ 1:100 dilution of the virus in line #3.

Animals were inoculated within 3 hours of preparing the syringes for dosing; vaccines were kept on wet ice until dosing. Any remaining vaccine material was returned to the designated storage temperature (but not reused) and shipped back to the sponsor at the end of the study.

### Reference Yellow Fever Vaccine Virus (YF 17D) Preparation

Lyophilized yellow fever 17D vaccine virus reference batch YF 17D/168-73 was obtained from NIBSC. Ampule vials were removed from a freezer and allowed to acclimate to room temperature (about 10 minutes). The ampule was gently tapped to collect the material at the bottom (labeled) end. Once the ampule had been opened, the contents of ampule were reconstituted in 0.5 mL WFI as described in NIBSC product information sheet, then further diluted 1 to 5 with vaccine diluent (50/50 DMEM and OptiPro), as indicated below, to achieve a target dose of 10⁵ PFU/0.5 mL per animal.

| | | Dose | | Total Inoculum | Reconstitution | Diluent | Volume/ |
|---|---|---|---|---|---|---|---|
| Group | Vaccine Name | (PFU) | N + 1 | Volume (mL) | Volume (mL) | Volume (mL) | animal (mL) |
| 4 | YF 17D 168-73 | 10⁵ | 4 | 2.5 | 0.5 | 2.0 | 0.5 |

Each 1.0 mL syringe with 27-gauge needle was filled with 0.5 mL of YF 17D vaccine inoculum and kept on ice until transferred to animal facility for dosing. Any remaining vaccine material was retained and stored at −80° C. or below for shipping back to the sponsor.

### Blood Processing

Blood was processed following Standard Operating Procedures (SOPs) of Southern Research or manufacturer recommendations for centrifuge time, speed and temperature, aliquoted into appropriate vials and stored at −70° C. or below until shipment to the Sponsor.

### Results and Discussion

### Clinical Observations

Clinical observation data are summarized in the table below. All clinical observations were normal except for emesis recorded for a female in Group 4 (18163) on Days 4-6. The association of the observation to the reference vaccine is uncertain.

### Safety and Immunogenicity of a Live Attenuated Yellow Fever (YF) Vaccine in Rhesus Macaques

### Summary of Clinical Observations

| | Observation | Days(s) Relative to Start Date | | | | | | | | | | | | | |
|---|---|---|---|---|---|---|---|---|---|---|---|---|---|---|---|
| Sex: Both | Type: All types | 0 | 1 | 2 | 3 | 4 | 5 | 6 | 30 | 31 | 32 | 33 | 34 | 35 | 36 |
| 1 M/F | Normal | 3 | 3 | 3 | 3 | 3 | 3 | 3 | 3 | 3 | 3 | 3 | 3 | 3 | 3 |
| 2 M/F | Normal | 3 | 3 | 3 | 3 | 3 | 3 | 3 | 3 | 3 | 3 | 3 | 3 | 3 | 3 |
| 3 M/F | Normal | 3 | 3 | 3 | 3 | 3 | 3 | 3 | 3 | 3 | 3 | 3 | 3 | 3 | 3 |
| 4 M/F | Normal | 3 | 3 | 3 | 3 | 2 | 2 | 2 | 3 | 3 | 3 | 3 | 3 | 3 | 3 |
| | Emesis | — | — | — | — | 1 | 1 | 1 | — | — | — | — | — | — | — |

Key page

### Group Information

| Short | Long | | Report | | |
|---|---|---|---|---|---|
| Name | Name | Type | Headings | | |
| 1 | 1 M/F | Dose | 17D-WD | 10\hat{ }6 PFU | Dose |
| 2 | 2 M/F | Dose | 17D-WWDW | 10\hat{ }5 PFU | Dose |
| 3 | 3 M/F | Dose | 17D-WWDW | 10\hat{ }7 PFU | Dose |
| 4 | 4 M/F | Control | YF 17D | 5 log 10 | PFU Dose |

### Injection Site Monitoring

Injection sites appeared normal for all vaccinated animals and there were no adverse reactions recorded.

### In-Life Parameters

Body weight data are summarized in the table below.

### Safety and Immunogenicity of a Live Attenuated Yellow Fever (YF) Vaccine in Rhesus Macaques Summary of Body Weights

| | | Day(s) Relative to Start Date | | | | | | | |
|---|---|---|---|---|---|---|---|---|---|
| Sex: Both | | 0 | 2 | 4 | 6 | 30 | 32 | 34 | 46 |
| 17D-WD | Mean | 4.70 | 4.93 | 5.27 | 4.87 | 5.53 | 5.50 | 5.13 | 5.37 |
| 10\hat{ }6 PFU | SD | 0.52 | 0.59 | 0.67 | 0.64 | 0.40 | 0.36 | 0.61 | 0.76 |
| Dose | N | 3 | 3 | 3 | 3 | 3 | 3 | 3 | 3 |
| 17D-WWDW | Mean | 4.40 | 4.37 | 4.90 | 4.50 | 4.87 | 4.83 | 4.67 | 4.93 |
| 10\hat{ }5PFU | SD | 1.14 | 1.10 | 1.18 | 0.95 | 0.95 | 0.83 | 0.91 | 0.75 |
| Dose | N | 3 | 3 | 3 | 3 | 3 | 3 | 3 | 3 |
| 17D-WWDW | Mean | 4.80 | 4.80 | 5.43 | 4.83 | 5.17 | 5.30 | 5.10 | 5.20 |
| 10\hat{ }7 PFU | SD | 0.40 | 0.70 | 0.47 | 0.29 | 0.67 | 0.62 | 0.53 | 0.69 |
| Dose | N | 3 | 3 | 3 | 3 | 3 | 3 | 3 | 3 |
| YF 17D | Mean | 4.03 | 4.07 | 4.57 | 4.07 | 4.67 | 4.67 | 4.33 | 4.50 |
| 5 log10 | SD | 0.80 | 0.75 | 0.80 | 0.75 | 0.60 | 0.70 | 0.65 | 0.56 |
| Dose | N | 3 | 3 | 3 | 3 | 3 | 3 | 3 | 3 |

Body temperature data are summarized in the table below. All vaccines tested in the study were well tolerated in animals and no significant weight loss or fever was observed in any of the animals during the study.

All animals consistently ate all of their food daily during Days 0-6 and Days 30-36.

### Safety and Immunogenicity of a Live Attenuated Yellow Fever (YF) Vaccine in Rhesus Macaques

Summary of Body Temperature (Deg. F)

| | | Day(s) Relative to Start Date | | | | | | | |
|---|---|---|---|---|---|---|---|---|---|
| Sex: Both | | 0 | 2 | 4 | 6 | 30 | 32 | 34 | 46 |
| 17D-WD | Mean | 102.67 | 101.33 | 101.57 | 102.30 | 101.93 | 102.30 | 102.57 | 102.67 |
| 10\hat{ }6 PFU | SD | 0.93 | 0.35 | 1.29 | 0.26 | 1.07 | 0.40 | 0.15 | 0.81 |
| Dose | N | 3 | 3 | 3 | 3 | 3 | 3 | 3 | 3 |
| 17D-WWDW | Mean | 100.73 | 101.07 | 99.80 | 101.67 | 102.40 | 102.10 | 102.17 | 102.90 |
| 10\hat{ }5PFU | SD | 2.37 | 1.10 | 2.78 | 1.16 | 0.82 | 0.95 | 0.57 | 0.20 |
| Dose | N | 3 | 3 | 3 | 3 | 3 | 3 | 3 | 3 |
| 17D-WWDW | Mean | 101.73 | 100.73 | 100.57 | 101.87 | 100.47 | 100.23 | 101.93 | 101.93 |
| 10\hat{ }7 PFU | SD | 0.76 | 1.00 | 1.23 | 0.38 | 3.35 | 3.61 | 0.87 | 0.47 |
| Dose | N | 3 | 3 | 3 | 3 | 3 | 3 | 3 | 3 |
| YF 17D | Mean | 101.83 | 101.03 | 101.50 | 101.90 | 101.40 | 102.33 | 102.10 | 102.30 |
| 5 log10 | SD | 0.90 | 0.47 | 0.87 | 1.05 | 0.44 | 0.75 | 0.35 | 0.17 |
| Dose | N | 3 | 3 | 3 | 3 | 3 | 3 | 3 | 3 |

### CONCLUSIONS

This study was designed to evaluate the safety, attenuation and immunogenicity of two live attenuated yellow fever (YF) vaccine candidates (YF 17D-WD and YF 17D-WWDW) in Rhesus Macaques, as compared to YF 17D reference vaccine.

All animals received vaccines (17D-WD (10⁶ PFU), 17D-WWDW (low dose, 10⁵ PFU), 17D-WWDW (high dose, 10⁷ PFU) and YF 17D (10⁵ PFU) on Day 0 and 30 via SC injection. The in-life assessments demonstrated that YF-Vaccines 17D-WD, 17D-WWDW (low dose) and 17D-WWDW (high dose) were well tolerated with no adverse injection site reactions observed in any animal. Overall, there was no apparent difference in the in-life parameters between the YF-Vaccines 17D-WD and 17D-WWDW and the reference vaccine.

Serum for immunogenicity (PRNT) and viral load assessment (qRT-PCR) was collected as described in the study protocol.

Analysis of neutralizing antibodies by PRNT50 assay on serum collected on Days 21, 30, and 51 was conducted. Day 30 PRNT50 titers showed an average increase of 30- to 200-fold over baseline after one dose of vaccine. A second identical dose of each vaccine given on Day 30 further increased PRNT50 titers in all groups between 3 to 10-fold by Day 51, to as high as 300- to 800-fold above baseline. After both prime and boosting dose, candidate vaccine 17D-WWDW (at both 105 and 107 dose levels) produced levels of nAbs that were not statistically different from 17D WHO reference preparation 168-73 at a standard 105 PFU dose after either the prime or the boosting dose (p-values >0.05). Taken together, these data suggest that a single dose of either 10⁵ or 10⁷ PFU of vaccine candidate 17D-WWDW elicits similar immunogenicity to 17D.

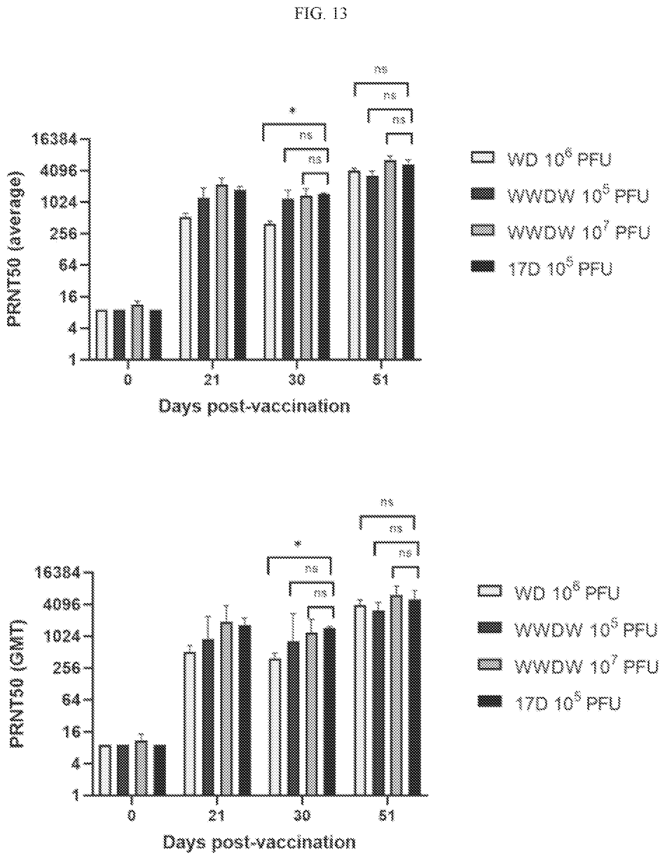

FIG. 13 depicts neutralizing antibody titers against 17D in monkey sera.

Levels of nAbs produced by candidate vaccine 17D-WD after the first dose were significantly lower than those of the 17D comparator (p=0.0001 at Day 30). 17D-WD titers rose after the second dose to levels not statistically different from those seen after 2 doses of 17D (p>0.05), suggesting that 17D-WD provided a stronger boost in antibody response than 17D.

Viremia analysis by qRT-PCR on samples collected on Days 2, 4, 6, 32, 24 and 36 was conducted. Viremia was detected in all animals on Day 2 at a low titer, which decreased and/or became undetectable in most animals by Day 6. 17D-WD and 17D-WWDW vaccinated animals had overall fewer days of viremia than 17D vaccinated animals. There was a trend of 17D-WD viremia dropping more rapidly, becoming undetectable in all animals on Day 6 post vaccination, suggesting that 17D-WD may be more attenuated than the 17D reference vaccine. However due to the small group sizes and spread of the values within the groups neither group mean viremia titers reached statistical significance relative to the reference vaccine group (p-values >0.05).

There was no detectable viremia in any animal on Days 32, 34, and 36 (2, 4, and 6 days after dose 2), suggesting that vaccinated animals were protected from the surrogate challenge posed by the vaccine booster dose.

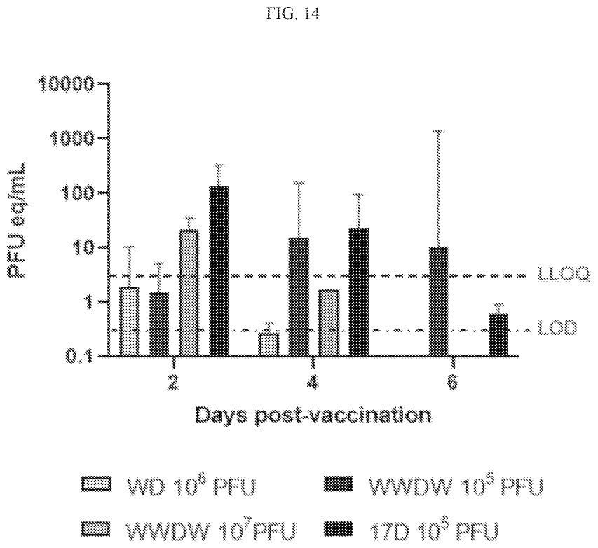

FIG. 14 depicts post-vaccination viremia.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A polynucleotide comprising a polynucleotide encoding one or more viral proteins or one or more fragments thereof of a parent Yellow Fever virus (YFV):
wherein the polynucleotide is recoded compared to its parent YFV,
wherein the amino acid sequence of the one or more viral proteins, or one or more fragments thereof of the parent YFV encoded by the polynucleotide remains the same, or
wherein the amino acid sequence of the one or more viral proteins or one or more fragments thereof of the parent YFV encoded by the polynucleotide comprises one or more amino acid substitutions, additions, or deletions.

2. The polynucleotide of claim 1, wherein the one or more viral proteins or one or more fragments thereof is the E protein or a fragment thereof.

3. The polynucleotide of claim 2, wherein the E protein or a fragment thereof is encoded by a polynucleotide having SEQ ID NOs:7, 3, 9, 8, 4, 5 or 6, or a fragment thereof, or
variant of a polynucleotide having SEQ ID NOs:7, 3, 9, 8, 4, 5 or 6, or a fragment thereof.

4. (canceled)

5. (canceled)

6. (canceled)

7. (canceled)

8. The polynucleotide of claim 1, wherein the polynucleotide sequence is SEQ ID NOs:7, 3, 9, 8, 4, 5 or 6.

9. The polynucleotide claim 1, wherein the parent YFV is YFV strain 17D (YFV 17D), or has at least 95%, 96%, 97%, 98%, or 99% sequence identity to YFV 17D, or wherein the parent YFV is YFV 17D-204, YFV 17DD, or YFV 17D-213, or has at least 95%, 96%, 97%, 98%, or 99% sequence identity to YFV 17D-204, YFV 17DD, or YFV 17D-213.

10. (canceled)

11. A polypeptide encoded by a polynucleotide of claim 1.

12. A deoptimized Yellow Fever virus (YFV) comprising a polynucleotide of claim 1.

13. A deoptimized Yellow Fever virus (YFV) comprising a polypeptide encoded by a polynucleotide of claim 1.

14. A deoptimized Yellow Fever virus (YFV) of claim 12, wherein expression of one or more of its viral proteins is reduced compared to its parent YFV.

15. An immune composition or vaccine composition comprising a deoptimized YFV of claim 12.

16. A method of treating a malignant tumor or reducing tumor size, comprising:
administering a deoptimized Yellow Fever virus (YFV) of claim 12, or an immune composition comprising a deoptimized YFV of claim 12 to a subject in need thereof.

17. A method of treating a malignant tumor or reducing tumor size, comprising:
administering a prime dose of deoptimized YFV of claim 12, or an immune composition comprising a deoptimized YFV of claim 12, to a subject in need thereof; and
administering one or more boost dose of deoptimized YFV of claim 12, or an immune composition comprising a deoptimized YFV of claim 12, to the subject in need thereof.

18. (canceled)

19. The method of claim 16, wherein the deoptimized YFV is deoptimized YFV strain 17D (YFV 17D), or wherein the deoptimized YFV is deoptimized YFV 17D-204, deoptimized YFV 17DD, or deoptimized YFV 17D-213.

20. (canceled)

21. (canceled)

22. (canceled)

23. (canceled)

24. (canceled)

25. (canceled)

26. (canceled)

27. (canceled)

28. (canceled)

29. The method of claim 16, wherein the method further comprises administering a PD-1 inhibitor or a PD-L1 inhibitor.

30. (canceled)

31. (canceled)

32. (canceled)

33. (canceled)

34. The method of claim 16, wherein treating the malignant tumor stimulates an inflammatory immune response in the tumor, or wherein treating the malignant tumor recruits pro-inflammatory cells to the tumor, or wherein treating the malignant tumor stimulates an anti-tumor immune response.

35. (canceled)

36. (canceled)

37. (canceled)

38. The method of claim 16, wherein the malignant tumor is selected from a group consisting of glioma, neuroblastoma, glioblastoma multiforme, adenocarcinoma, medulloblastoma, mammary carcinoma, prostate carcinoma, colorectal carcinoma, hepatocellular carcinoma, bladder cancer, prostate cancer, lung carcinoma, bronchial carcinoma, epidermoid carcinoma, and melanoma.

39. The method of claim 16, wherein the deoptimized YFV is administered intratumorally, intravenously, intracerebrally, intramuscularly, intraspinally or intrathecally.

40. The method of claim 16, wherein administering the deoptimized YFV causes cell lysis in the tumor cells.

41. A method of eliciting an immune response in a subject in need thereof, comprising:
administering a deoptimized Yellow Fever virus (YFV) of claim 12, or an immune or vaccine composition comprising a deoptimized YFV of claim 12, to a subject in need thereof.

42. A method of eliciting an immune response in a subject, comprising:
administering a prime dose of deoptimized YFV of claim 12, or an immune or vaccine composition comprising a deoptimized YFV of claim 12, to a subject in need thereof; and administering one or more boost dose of deoptimized YFV of claim 12, or an immune or vaccine composition comprising a deoptimized YFV of claim 12, to the subject in need thereof.

43. (canceled)

44. (canceled)

45. (canceled)


SEQUENCE LISTING (42 sequences)

SEQ ID NO: 1 - DNA, 10862 bp, Yellow fever virus
  source 1..10862: mol_type = genomic DNA
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaccttcaagaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggaatgctgttgatgacgggtggagtgaccttggtgcggaaaaacagatggttgctcctaaatgtgacatctgaggacctcgggaaaacattctctgtgggcacaggcaactgcacaacaaacattttggaagccaagtactggtgcccagactcaatggaatacaactgtcccaatctcagtccaagagaggagccagatgacattgattgctggtgctatggggtggaaaacgttagagtcgcatatggtaagtgtgactcagcaggcaggtctaggaggtcaagaagggccattgacttgcctacgcatgaaaaccatggtttgaagacccggcaagaaaaatggatgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcccttgagcatgagatgtggagaagcagggcagatgagatcaatgccatttttgaggaaaacgaggtggacatttctgttgtcgtgcaggatccaaagaatgtttaccagagaggaactcatccattttccagaattcgggatggtctgcagtatggttggaagacttggggtaagaaccttgtgttctccccagggaggaagaatggaagcttcatcatagatggaaagtccaggaaagaatgcccgttttcaaaccgggtctggaattctttccagatagaggagtttgggacgggagtgttcaccacacgcgtgtacatggacgcagtctttgaatacaccatagactgcgatggatctatcttgggtgcagcggtgaacggaaaaaagagtgcccatggctctccaacattttggatgggaagtcatgaagtaaatgggacatggatgatccacaccttggaggcattagattacaaggagtgtgagtggccactgacacatacgattggaacatcagttgaagagagtgaaatgttcatgccgagatcaatcggaggcccagttagctctcacaatcatatccctggatacaaggttcagacgaacggaccttggatgcaggtaccactagaagtgaagagagaagcttgcccagggactagcgtgatcattgatggcaactgtgatggacggggaaaatcaaccagatccaccacggatagcgggaaagttattcctgaatggtgttgccgctcctgcacaatgccgcctgtgagcttccatggtagtgatgggtgttggtatcccatggaaattaggccaaggaaaacgcatgaaagccatctggtgcgctcctgggttacagctggagaaatacatgctgtcccttttggtttggtgagcatgatgatagcaatggaagtggtcctaaggaaaagacagggaccaaagcaaatgttggttggaggagtagtgctcttgggagcaatgctggtcgggcaagtaactctccttgatttgctgaaactcacagtggctgtgggattgcatttccatgagatgaacaatggaggagacgccatgtatatggcgttgattgctgccttttcaatcagaccagggctgctcatcggctttgggctcaggaccctatggagccctcgggaacgccttgtgctgaccctaggagcagccatggtggagattgccttgggtggcgtgatgggcggcctgtggaagtatctaaatgcagtttctctctgcatcctgacaataaatgctgttgcttctaggaaagcatcaaataccatcttgcccctcatggctctgttgacacctgtcactatggctgaggtgagacttgccgcaatgttcttttgtgccgtggttatcataggggtccttcaccagaatttcaaggacacctccatgcagaagactatacctctggtggccctcacactcacatcttacctgggcttgacacaaccttttttgggcctgtgtgcatttctggcaacccgcatatttgggcgaaggagtatcccagtgaatgaggcactcgcagcagctggtctagtgggagtgctggcaggactggcttttcaggagatggagaacttccttggtccgattgcagttggaggactcctgatgatgctggttagcgtggctgggagggtggatgggctagagctcaagaagcttggtgaagtttcatgggaagaggaggcggagatcagcgggagttccgcccgctatgatgtggcactcagtgaacaaggggagttcaagctgctttctgaagagaaagtgccatgggaccaggttgtgatgacctcgctggccttggttggggctgccctccatccatttgctcttctgctggtccttgctgggtggctgtttcatgtcaggggagctaggagaagtggggatgtcttgtgggatattcccactcctaagatcatcgaggaatgtgaacatctggaggatgggatttatggcatattccagtcaaccttcttgggggcctcccagcgaggagtgggagtggcacagggaggggtgttccacacaatgtggcatgtcacaagaggagctttccttgtcaggaatggcaagaagttgattccatcttgggcttcagtaaaggaagaccttgtcgcctatggtggctcatggaagttggaaggcagatgggatggagaggaagaggtccagttgatcgcggctgttccaggaaagaacgtggtcaacgtccagacaaaaccgagcttgttcaaagtgaggaatgggggagaaatcggggctgtcgctcttgactatccgagtggcacttcaggatctcctattgttaacaggaacggagaggtgattgggctgtacggcaatggcatccttgtcggtgacaactccttcgtgtccgccatatcccagactgaggtgaaggaagaaggaaaggaggagctccaagagatcccgacaatgctaaagaaaggaatgacaactgtccttgattttcatcctggagctgggaagacaagacgtttcctcccacagatcttggccgagtgcgcacggagacgcttgcgcactcttgtgttggcccccaccagggttgttctttctgaaatgaaggaggcttttcacggcctggacgtgaaattccacacacaggctttttccgctcacggcagcgggagagaagtcattgatgctatgtgccatgccaccctaacttacaggatgttggaaccaactagggttgttaactgggaagtgatcattatggatgaagcccattttttggatccagctagcatagccgctagaggttgggcagcgcacagagctagggcaaatgaaagtgcaacaatcttgatgacagccacaccgcctgggactagtgatgaatttccacattcaaatggtgaaatagaagatgttcaaacggacatacccagtgagccctggaacacagggcatgactggatcctggctgacaaaaggcccacggcatggttccttccatccatcagagctgcaaatgtcatggctgcctctttgcgtaaggctggaaagagtgtggtggtcctgaacaggaaaacctttgagagagaataccccacgataaagcagaagaaacctgactttatattggccactgacatagctgaaatgggagccaacctttgcgtggagcgagtgctggattgcaggacggcttttaagcctgtgcttgtggatgaagggaggaaggtggcaataaaagggccacttcgtatctccgcatcctctgctgctcaaaggagggggcgcattgggagaaatcccaacagagatggagactcatactactattctgagcctacaagtgaaaataatgcccaccacgtctgctggttggaggcctcaatgctcttggacaacatggaggtgaggggtggaatggtcgccccactctatggcgttgaaggaactaaaacaccagtttcccctggtgaaatgagactgagggatgaccagaggaaagtcttcagagaactagtgaggaattgtgacctgcccgtttggctttcgtggcaagtggccaaggctggtttgaagacgaatgatcgtaagtggtgttttgaaggccctgaggaacatgagatcttgaatgacagcggtgaaacagtgaagtgcagggctcctggaggagcaaagaagcctctgcgcccaaggtggtgtgatgaaagggtgtcatctgaccagagtgcgctgtctgaatttattaagtttgctgaaggtaggaggggagctgctgaagtgctagttgtgctgagtgaactccctgatttcctggctaaaaaaggtggagaggcaatggataccatcagtgtgtttctccactctgaggaaggctctagggcttaccgcaatgcactatcaatgatgcctgaggcaatgacaatagtcatgctgtttatactggctggactactgacatcgggaatggtcatctttttcatgtctcccaaaggcatcagtagaatgtctatggcgatgggcacaatggccggctgtggatatctcatgttccttggaggcgtcaaacccactcacatctcctatatcatgctcatattctttgtcctgatggtggttgtgatccccgagccagggcaacaaaggtccatccaagacaaccaagtggcatacctcattattggcatcctgacgctggtttcagcggtggcagccaacgagctaggcatgctggagaaaaccaaagaggacctctttgggaagaagaacttaattccatctagtgcttcaccctggagttggccggatcttgacctgaagccaggagctgcctggacagtgtacgttggcattgttacaatgctctctccaatgttgcaccactggatcaaagtcgaatatggcaacctgtctctgtctggaatagcccagtcagcctcagtcctttctttcatggacaaggggataccattcatgaagatgaatatctcggtcataatgctgctggtcagtggctggaattcaataacagtgatgcctctgctctgtggcatagggtgcgccatgctccactggtctctcattttacctggaatcaaagcgcagcagtcaaagcttgcacagagaagggtgttccatggcgttgccaagaaccctgtggttgatgggaatccaacagttgacattgaggaagctcctgaaatgcctgccctttatgagaagaaactggctctatatctccttcttgctctcagcctagcttctgttgccatgtgcagaacgcccttttcattggctgaaggcattgtcctagcatcagctgccctagggccgctcatagagggaaacaccagccttctttggaatggacccatggctgtctccatgacaggagtcatgagggggaatcactatgcttttgtgggagtcatgtacaatctatggaagatgaaaactggacgccgggggagcgcgaatggaaaaactttgggtgaagtctggaagagggaactgaatctgttggacaagcgacagtttgagttgtataaaaggaccgacattgtggaggtggatcgtgatacggcacgcaggcatttggccgaagggaaggtggacaccggggtggcggtctccagggggaccgcaaagttaaggtggttccatgagcgtggctatgtcaagctggaaggtagggtgattgacctggggtgtggccgcggaggctggtgttactacgctgctgcgcaaaaggaagtgagtggggtcaaaggatttactcttggaagagacggccatgagaaacccatgaatgtgcaaagtctgggatggaacatcatcaccttcaaggacaaaactgatatccaccgcctagaaccagtgaaatgtgacacccttttgtgtgacattggagagtcatcatcgtcatcggtcacagagggggaaaggaccgtgagagttcttgatactgtagaaaaatggctggcttgtggggttgacaacttctgtgtgaaggtgttagctccatacatgccagatgttctcgagaaactggaattgctccaaaggaggtttggcggaacagtgatcaggaaccctctctccaggaattccactcatgaaatgtactacgtgtctggagcccgcagcaatgtcacatttactgtgaaccaaacatcccgcctcctgatgaggagaatgaggcgtccaactggaaaagtgaccctggaggctgacgtcatcctcccaattgggacacgcagtgttgagacagacaagggacccctggacaaagaggccatagaagaaagggttgagaggataaaatctgagtacatgacctcttggttttatgacaatgacaacccctacaggacctggcactactgtggctcctatgtcacaaaaacctcaggaagtgcggcgagcatggtaaatggtgttattaaaattctgacatatccatgggacaggatagaggaggtcacaagaatggcaatgactgacacaaccccttttggacagcaaagagtgtttaaagaaaaagttgacaccagagcaaaggatccaccagcgggaactaggaagatcatgaaagttgtcaacaggtggctgttccgccacctggccagagaaaagaaccccagactgtgcacaaaggaagaatttattgcaaaagtccgaagtcatgcagccattggagcttacctggaagaacaagaacagtggaagactgccaatgaggctgtccaagacccaaagttctgggaactggtggatgaagaaaggaagctgcaccaacaaggcaggtgtcggacttgtgtgtacaacatgatggggaaaagagagaagaagctgtcagagtttgggaaagcaaagggaagccgtgccatatggtatatgtggctgggagcgcggtatcttgagtttgaggccctgggattcctgaatgaggaccattgggcttccagggaaaactcaggaggaggagtggaaggcattggcttacaatacctaggatatgtgatcagagacctggctgcaatggatggtggtggattctacgcggatgacaccgctggatgggacacgcgcatcacagaggcagaccttgatgatgaacaggagatcttgaactacatgagcccacatcacaaaaaactggcacaagcagtgatggaaatgacatacaagaacaaagtggtgaaagtgttgagaccagccccaggagggaaagcctacatggatgtcataagtcgacgagaccagagaggatccgggcaggtagtgacttatgctctgaacaccatcaccaacttgaaagtccaattgatcagaatggcagaagcagagatggtgatacatcaccaacatgttcaagattgtgatgaatcagttctgaccaggctggaggcatggctcactgagcacggatgtaacagactgaagaggatggcggtgagtggagacgactgtgtggtccggcccatcgatgacaggttcggcctggccctgtcccatctcaacgccatgtccaaggttagaaaggacatatctgaatggcagccatcaaaagggtggaatgattgggagaatgtgcccttctgttcccaccacttccatgaactacagctgaaggatggcaggaggattgtggtgccttgccgagaacaggacgagctcattgggagaggaagggtgtctccaggaaacggctggatgatcaaggaaacagcttgcctcagcaaagcctatgccaacatgtggtcactgatgtattttcacaaaagggacatgaggctactgtcattggctgtttcctcagctgttcccacctcatgggttccacaaggacgcacaacatggtcgattcatgggaaaggggagtggatgaccacggaagacatgcttgaggtgtggaacagagtatggataaccaacaacccacacatgcaggacaagacaatggtgaaaaaatggagagatgtcccttatctaaccaagagacaagacaagctgtgcggatcactgattggaatgaccaatagggccacctgggcctcccacatccatttggtcatccatcgtatccgaacgctgattggacaggagaaatacactgactacctaacagtcatggacaggtattctgtggatgctgacctgcaactgggtgagcttatctgaaacaccatctaacaggaataaccgggatacaaaccacgggtggagaaccggactccccacaacctgaaaccgggatataaaccacggctggagaaccggactccgcacttaaaatgaaacagaaaccgggataaaaactacggatggagaaccggactccacacattgagacagaagaagttgtcagcccagaaccccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccagaggtctgtgagcacagtttgctcaagaataagcagacctttggatgacaaacacaaaaccact

SEQ ID NO: 2 - DNA, 1479 bp, Yellow oat-grass mosaic virus
  source 1..1479: mol_type = other DNA
gctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcg

SEQ ID NO: 3 - DNA, 1479 bp, synthetic construct
  source 1..1479: mol_type = other DNA
gctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgct

SEQ ID NO: 4 - DNA, 1479 bp, synthetic construct
  source 1..1479: mol_type = other DNA
gctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcg

SEQ ID NO: 5 - DNA, 1479 bp, synthetic construct
  source 1..1479: mol_type = other DNA
gctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgct

SEQ ID NO: 6 - DNA, 1479 bp, synthetic construct
  source 1..1479: mol_type = other DNA
gctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcg

SEQ ID NO: 7 - DNA, 1479 bp, synthetic construct
  source 1..1479: mol_type = other DNA
gctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcg

SEQ ID NO: 8 - DNA, 1479 bp, synthetic construct
  source 1..1479: mol_type = other DNA
gctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggaataccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgct

SEQ ID NO: 9 - DNA, 1479 bp, synthetic construct
  source 1..1479: mol_type = other DNA
gctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggaataccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcg

SEQ ID NO: 10 - protein, 493 aa, synthetic construct
AHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWTTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGAVLIWVGINTRNMTMSMSMILVGVIMMFLSLGVGA

SEQ ID NO: 11 - protein, 493 aa, synthetic construct
AHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGAVLIWVGINTRNMTMSMSMILVGVIMMFLSLGVGA

SEQ ID NO: 12 - DNA, 1794 bp, synthetic construct
  source 1..1794: mol_type = other DNA
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 13 - DNA, 1794 bp, synthetic construct
  source 1..1794: mol_type = other DNA
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 14 - DNA, 1794 bp, synthetic construct
  source 1..1794: mol_type = other DNA
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 15 - DNA, 1794 bp, synthetic construct
  source 1..1794: mol_type = other DNA
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 16 - DNA, 1794 bp, synthetic construct
  source 1..1794: mol_type = other DNA
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 17 - DNA, 27 bp, synthetic construct
  source 1..27: mol_type = other DNA
agtaaatcctgtgtgctaattgaggtg

SEQ ID NO: 18 - DNA, 24 bp, synthetic construct
  source 1..24: mol_type = other DNA
tgtcagtaattccaatgcagtgag

SEQ ID NO: 19 - DNA, 26 bp, synthetic construct
  source 1..26: mol_type = other DNA
agcttatcatcgataagcttgctagc

SEQ ID NO: 20 - DNA, 24 bp, synthetic construct
  source 1..24: mol_type = other DNA
tgtcagtaattccaatgcagtgag

SEQ ID NO: 21 - DNA, 24 bp, synthetic construct
  source 1..24: mol_type = other DNA
atgactggaagaatgggtgaaagg

SEQ ID NO: 22 - DNA, 25 bp, synthetic construct
  source 1..25: mol_type = other DNA
agaggctttcactattgatgcaagc

SEQ ID NO: 23 - DNA, 24 bp, synthetic construct
  source 1..24: mol_type = other DNA
atcaaggatgcgccatcaactttg

SEQ ID NO: 24 - DNA, 24 bp, synthetic construct
  source 1..24: mol_type = other DNA
aagtctcacctcagccatagtgac

SEQ ID NO: 25 - DNA, 21 bp, synthetic construct
  source 1..21: mol_type = other DNA
aacgccttgtgctgaccctag

SEQ ID NO: 26 - DNA, 25 bp, synthetic construct
  source 1..25: mol_type = other DNA
ttggttccaacatcctgtaagttag

SEQ ID NO: 27 - DNA, 20 bp, synthetic construct
  source 1..20: mol_type = other DNA
atcttggccgagtgcgcacg

SEQ ID NO: 28 - DNA, 21 bp, synthetic construct
  source 1..21: mol_type = other DNA
tcggggatcacaaccaccatc

SEQ ID NO: 29 - DNA, 24 bp, synthetic construct
  source 1..24: mol_type = other DNA
tgctgtttatactggctggactac

SEQ ID NO: 30 - DNA, 22 bp, synthetic construct
  source 1..22: mol_type = other DNA
tggcatgtatggagctaacacc

SEQ ID NO: 31 - DNA, 25 bp, synthetic construct
  source 1..25: mol_type = other DNA
atcatcaccttcaaggacaaaactg

SEQ ID NO: 32 - DNA, 21 bp, synthetic construct
  source 1..21: mol_type = other DNA
atccgtgctcagtgagccatg

SEQ ID NO: 33 - DNA, 23 bp, synthetic construct
  source 1..23: mol_type = other DNA
agcctacatggatgtcataagtc

SEQ ID NO: 34 - DNA, 26 bp, synthetic construct
  source 1..26: mol_type = other DNA
agtggttttgtgtttgtcatccaaag

SEQ ID NO: 35 - DNA, 22 bp, synthetic construct
  source 1..22: mol_type = other DNA
acgtgaaattccacacacaggc

SEQ ID NO: 36 - DNA, 22 bp, synthetic construct
  source 1..22: mol_type = other DNA
acttggttgtcttggatggacc

SEQ ID NO: 37 - DNA, 24 bp, synthetic construct
  source 1..24: mol_type = other DNA
tgctgtttatactggctggactac

SEQ ID NO: 38 - DNA, 22 bp, synthetic construct
  source 1..22: mol_type = other DNA
tggcatgtatggagctaacacc

SEQ ID NO: 39 - DNA, 25 bp, synthetic construct
  source 1..25: mol_type = other DNA
atcatcaccttcaaggacaaaactg

SEQ ID NO: 40 - DNA, 22 bp, synthetic construct
  source 1..22: mol_type = other DNA
tgttacatccgtgctcagtgag

SEQ ID NO: 41 - DNA, 23 bp, synthetic construct
  source 1..23: mol_type = other DNA
agcctacatggatgtcataagtc

SEQ ID NO: 42 - DNA, 32 bp, synthetic construct
  source 1..32: mol_type = other DNA
agtggttttgtgtttgtcatccaaaggtctgc